SEQUENCE LISTING (35 sequences)

SEQ ID NO: 1 - protein, 498 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADFSG

SEQ ID NO: 2 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 3 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 4 - protein, 129 aa, Artificial Sequence
  variable heavy chain domain VH of <ANG-2> LC06
QVQLVESGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSS

SEQ ID NO: 5 - protein, 110 aa, Artificial Sequence
  variable light chain domain VL of <ANG-2> LC06 and E6Q
QPGLTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVLGQ

SEQ ID NO: 6 - protein, 117 aa, Artificial Sequence
  variable heavy chain domain VH of <VEGF> B20.4.1
EVQLVESGGGLVQPGGSLRLSCAASGFSINGSWIFWVRQAPGKGLEWVGAIWPFGGYTHYADSVKGRFTISADTSKNTAYLQMNSLRAEDTAVYYCARWGHSTSPWAMDYWGQGTLV

SEQ ID NO: 7 - protein, 108 aa, Artificial Sequence
  variable light chain domain VL of <VEGF> B20.4.1
DIQMTQSPSSLSASVGDRVTITCRASQVIRRSLAWYQQKPGKAPKLLIYAASNLASGVPSRFSGSGSGTDFTLTISSLQPEDFATYYCQQSNTSPLTFGQGTKVEIKR

SEQ ID NO: 8 - protein, 123 aa, Artificial Sequence [sequence omitted]

SEQ ID NO: 9 - protein, 107 aa, Artificial Sequence [sequence omitted]

SEQ ID NO: 10 - protein, 128 aa, Artificial Sequence
  variable heavy chain domain VH of <ANG-2> E6Q
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVS

SEQ ID NO: 11 - protein, 461 aa, Artificial Sequence
  ANG-2 heavy chain of <ANG-2/VEGF> LC06/B20.4.1 and <ANG-2/VEGF> LC06/bevacizumab
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 12 - protein, 213 aa, Artificial Sequence
  ANG-2 light chain of <ANG-2/VEGF> LC06/B20.4.1 and <ANG-2/VEGF> LC06/bevacizumab
QPGLTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - protein, 451 aa, Artificial Sequence
  VEGF heavy chain of <ANG-2/VEGF> LC06/B20.4.1
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 14 - protein, 214 aa, Artificial Sequence
  VEGF light chain of <ANG-2/VEGF> LC06/B20.4.1
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 451 aa, Artificial Sequence; identical to SEQ ID NO: 13
  VEGF heavy chain of <ANG-2/VEGF> LC06/ bevacizumab

SEQ ID NO: 16 - protein, 214 aa, Artificial Sequence; identical to SEQ ID NO: 14
  VEGF light chain of <ANG-2/VEGF> LC06/ bevacizumab

SEQ ID NO: 17 - protein, 118 aa, Artificial Sequence
  variable heavy chain domain VH of <PD-L1> 243.55 variant 1
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 18 - protein, 118 aa, Artificial Sequence; identical to SEQ ID NO: 17
  variable heavy chain domain VH of <PD-L1> 243.55 variant 2

SEQ ID NO: 19 - protein, 118 aa, Artificial Sequence
  variable heavy chain domain VH of <PD-L1> 243.55 variant 3
EVQLVESGGGLVQPGGSLRLSCAASGFTFSGSWIHWVRQAPGKGLEWVAWILPYGGSSYYADSVKGRFTISADTSKNTAYLQMNSLRAEDTAVYYCARRHWPGGFDYWGQGTLVTVSA

SEQ ID NO: 20 - protein, 108 aa, Artificial Sequence
  variable light chain domain VL of <PD-L1> 243.55 variant 1
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 21 - protein, 108 aa, Artificial Sequence
  variable light chain domain VL of <PD-L1> 243.55 variant 2
DIQMTQSPSSLSASVGDRVTITCRASQDVSTAVAWYQQKPGKAPKLLIYSASFLYSGVPSRFSGSGSGTDFTLTISSLQPEDFATYYCQQYYNVPWTFGQGTKVEIKR

SEQ ID NO: 22 - protein, 108 aa, Artificial Sequence
  variable light chain domain VL of <PD-L1> 243.55 variant 3
DIQMTQSPSSLSASVGDRVTITCRASQDVSTAVAWYQQKPGKAPKLLIYSASFLYSGVPSRFSGSGSGTDFTLTISSLQPEDFATYYCQQYYAPPWTFGQGTKVEIKR

SEQ ID NO: 23 - protein, 108 aa, Artificial Sequence
  variable light chain domain VL of <PD-L1> 243.55 variant 4
DIQMTQSPSSLSASVGDRVTITCRASQDVSTAVAWYQQKPGKAPKLLIYSASFLYSGVPSRFSGSGSGTDFTLTISSLQPEDFATYYCQQYYTVPWTFGQGTKVEIKR

SEQ ID NO: 24 - protein, 108 aa, Artificial Sequence
  variable light chain domain VL of <PD-L1> 243.55 variant 5
DIQMTQSPSSLSASVGDRVTITCRASQVINTFLAWYQQKPGKAPKLLIYSASTLASGVPSRFSGSGSGTDFTLTISSLQPEDFATYYCQQYYTVPRTFGQGTKVEIKR

SEQ ID NO: 25 - protein, 108 aa, Artificial Sequence
  variable light chain domain VL of <PD-L1> 243.55 variant 6
DIQMTQSPSSLSASVGDRVTITCRASQDVSTAVAWYQQKPGKAPKLLIYSASFLYSGVPSRFSGSGSGTDFTLTISSLQPEDFATYYCQQGYGVPRTFGQGTKVEIKR

SEQ ID NO: 26 - protein, 108 aa, Artificial Sequence
  variable light chain domain VL of <PD-L1> 243.55 variant 7
DIQMTQSPSSLSASVGDRVTITCRASQDVSTAVAWYQQKPGKAPKLLIYSASFLYSGVPSRFSGSGSGTDFTLTISSLQPEDFATYYCQQYLFTPPTFGQGTKVEIKR

SEQ ID NO: 27 - protein, 108 aa, Artificial Sequence
  variable light chain domain VL of <PD-L1> 243.55 variant 8
DIQMTQSPSSLSASVGDRVTITCRASQDVSTAVAWYQQKPGKAPKLLIYSASFLYSGVPSRFSGSGSGTDFTLTISSLQPEDFATYYCQQYFITPTTFGQGTKVEIKR

SEQ ID NO: 28 - protein, 108 aa, Artificial Sequence
  variable light chain domain VL of <PD-L1> 243.55 variant 9
DIQMTQSPSSLSASVGDRVTITCRASQDVSTAVAWYQQKPGKAPKLLIYSASFLYSGVPSRFSGSGSGTDFTLTISSLQPEDFATYYCQQYYYTPPTFGQGTKVEIKR

SEQ ID NO: 29 - protein, 108 aa, Artificial Sequence
  variable light chain domain VL of <PD-L1> 243.55 variant 10
DIQMTQSPSSLSASVGDRVTITCRASQDVSTAVAWYQQKPGKAPKLLIYSASFLYSGVPSRFSGSGSGTDFTLTISSLQPEDFATYYCQQFFYTPPTFGQGTKVEIKR

SEQ ID NO: 30 - protein, 108 aa, Artificial Sequence
  variable light chain domain VL of <PD-L1> 243.55 variant 11
DIQMTQSPSSLSASVGDRVTITCRASQDVSTAVAWYQQKPGKAPKLLIYSASFLYSGVPSRFSGSGSGTDFTLTISSLQPEDFATYYCQQSLFTPPTFGQGTKVEIKR

SEQ ID NO: 31 - protein, 108 aa, Artificial Sequence
  variable light chain domain VL of <PD-L1> 243.55 variant 12
DIQMTQSPSSLSASVGDRVTITCRASQDVSTAVAWYQQKPGKAPKLLIYSASFLYSGVPSRFSGSGSGTDFTLTISSLQPEDFATYYCQQSLYTPPTFGQGTKVEIKR

SEQ ID NO: 32 - protein, 108 aa, Artificial Sequence
  variable light chain domain VL of <PD-L1> 243.55 variant 13
DIQMTQSPSSLSASVGDRVTITCRASQDVSTAVAWYQQKPGKAPKLLIYSASFLYSGVPSRFSGSGSGTDFTLTISSLQPEDFATYYCQQSWYHPPTFGQGTKVEIKR

SEQ ID NO: 33 - protein, 108 aa, Artificial Sequence
  variable light chain domain VL of <PD-L1> 243.55 variant 14
DIQMTQSPSSLSASVGDRVTITCRASQDVSTAVAWYQQKPGKAPKLLIYSASFLYSGVPSRFSGSGSGTDFTLTISSLQPEDFATYYCQQYFYIPPTFGQGTKVEIKR

SEQ ID NO: 34 - protein, 108 aa, Artificial Sequence
  variable light chain domain VL of <PD-L1> 243.55 variant 15
DIQMTQSPSSLSASVGDRVTITCRASQDVSTAVAWYQQKPGKAPKLLIYSASFLYSGVPSRFSGSGSGTDFTLTISSLQPEDFATYYCQQYWYTPTTFGQGTKVEIKR

SEQ ID NO: 35 - protein, 108 aa, Artificial Sequence
  variable light chain domain VL of <PD-L1> 243.55 variant 16
DIQMTQSPSSLSASVGDRVTITCRASQDVSTAVAWYQQKPGKAPKLLIYSASFLYSGVPSRFSGSGSGTDFTLTISSLQPEDFATYYCQQSYFIPPTFGQGTKVEIKR

(19) United States
(12) Patent Application Publication
(10) Pub. No.: US 2018/0155431 A1
(43) Pub. Date: Jun. 7, 2018

(54) COMBINATION THERAPY OF ANTIBODY BINDING TO ANGIOPOIETIN 2 WITH ANTIBODY BINDING TO PROGRAMMED DEATH LIGAND 1

(71) Applicant: Hoffmann-La Roche Inc., Little Falls, NJ (US)

(72) Inventors: Frank Herting, Penzberg (DE); Hans-Joachim Mueller, Penzberg (DE)

(73) Assignee: Hoffmann-La Roche Inc., Little Falls, NJ (US)

(21) Appl. No.: 15/788,070

(22) Filed: Oct. 19, 2017

Related U.S. Application Data
(63) Continuation of application No. PCT/EP2016/058870, filed on Apr. 21, 2016.

(30) Foreign Application Priority Data
Apr. 23, 2015 (EP) 15164803.7

(51) Int. Cl. C07K 16/28; A61P 35/00; C07K 16/22; C07K 16/30; A61K 39/00

(52) U.S. Cl. CPC C07K 16/2827; A61P 35/00; C07K 16/22; C07K 16/30; C07K 2317/31; C07K 2317/33; C07K 2317/56; C07K 2317/76; A61K 2039/507

(57) ABSTRACT

The present invention relates to a combination therapy of an antibody specifically binding to Angiopoietin 2 (ANG-2), and an antibody specifically binding to VEGF with an antibody specifically binding to programmed death ligand 1 (PD-L1).

### CROSS-REFERENCE TO RELATED APPLICATIONS

This application is a continuation of International Application No. PCT/EP2016/058870 having an International filing date of Apr. 21, 2016, the entire contents of which are incorporated herein by reference, and which claims the benefit of priority under 35 U.S.C. § 119 to EP 15164803.7, filed Apr. 23, 2015.

### SEQUENCE LISTING

The instant application contains a Sequence Listing submitted via EFS-Web and hereby incorporated by reference in its entirety. Said ASCII copy, created on Oct. 18, 2017, is named P32845-US SequenceListing.txt, and is 55,602 bytes in size.

### FIELD OF THE INVENTION

The present invention relates to a combination therapy of an antibody specifically binding to Angiopoietin 2 (ANG-2) with an antibody specifically binding to programmed death ligand 1 (PD-L1).

### BACKGROUND OF THE INVENTION

Angiopoietin 2

Angiopoietins, which play a key role in angiogenesis and blood vessel remodeling, are part of the pro-angiogenic armamentarium of growing tumors. Importantly they are one of the major factors leading to secondary resistance during anti-VEGF therapy (Saharinen, P., et al., Trends Mol Med 17 (2011) 347-362). Both angiopoietin-1 (ANG-1) and angiopoietin-2 (ANG-2) are Tie2-receptor ligands. While ANG-1 tends to stabilize and matures blood vessel (Yancopoulos, G. D., et al., Nature 407 (2000) 242-248), ANG-2 promotes tumor angiogenesis and growth by destabilizing blood vessels. ANG-2 thereby opposes ANG-1 in its function (Cascone, T. et al, J Clin Oncol 30 (2012) 441-444). Along this line it has been observed that blocking ANG-2, but not ANG-1 normalizes tumor blood vessels (Falcon, B. L., H. Hashizume, et al., Am J Pathol 175 (2009) 2159-2170) and helps to overcome acquired resistance towards anti-VEGF therapy (Chae, S. S., W. S. Kamoun, et al., Clin Cancer Res 16 (2010) 3618-3627; Thomas, M., et al. PLoS One 8 (2013) e54923). WO 2010/069532 relates to ANG-2 antibodies.

Therapeutic use of ANG-2 antibodies for treatment of cancer is also disclosed, e.g. in WO 2010/069532.

VEGF

Human vascular endothelial growth factor (VEGF/VEGF-A) is described in e.g. Leung, D. W., et al., Science 246 (1989) 1306-9; Keck, P. J., et al., Science 246 (1989) 1309-12 and Connolly, D. T., et al., J. Biol. Chem. 264 (1989) 20017-24. VEGF is involved in the regulation of normal and abnormal angiogenesis and neovascularization associated with tumors and intraocular disorders (Ferrara, N., et al., Endocr. Rev. 18 (1997) 4-25; Berkman, R. A., et al., J. Clin. Invest. 91 (1993) 153-159; Brown, L. F., et al., Human Pathol. 26 (1995) 86-91; Brown, L. F., et al., Cancer Res. 53 (1993) 4727-4735; Mattern, J., et al., Brit. J. Cancer. 73 (1996) 931-934; and Dvorak, H. F., et al., Am. J. Pathol. 146 (1995) 1029-1039). VEGF is a homodimeric glycoprotein that has been isolated from several sources. VEGF shows highly specific mitogenic activity for endothelial cells. VEGF has important regulatory functions in the formation of new blood vessels during embryonic vasculogenesis and in angiogenesis during adult life (Carmeliet, P., et al., Nature, 380 (1996) 435-439; Ferrara, N., et al., Nature, 380 (1996) 439-442; reviewed in Ferrara, N., et al., Endocr. Rev. 18 (1997) 4-25. Anti-VEGF neutralizing antibodies suppress the growth of a variety of human tumor cell lines in mice (Kim, K. J., et al., Nature 362 (1993) 841-844; Warren, S. R., et al., J. Clin. Invest. 95 (1995) 1789-1797; Borgstrom, P., et al., Cancer Res. 56 (1996) 4032-4039; and Melnyk, O., et al., Cancer Res. 56 (1996) 921-924).

WO 94/10202, WO 98/45332, WO 2005/00900 and WO 00/35956 refer to antibodies against VEGF. Humanized monoclonal antibody bevacizumab (sold under the trade name Avastin®) is an anti-VEGF antibody used in tumor therapy WO 98/45331).

WO 2010/040508 A9 and WO 2011/117329 relate to bispecific anti-VEGF/anti-ANG-2 antibodies and a therapeutic use thereof, e.g. for the treatment of cancer.

WO 2009/080253 and WO 2011/117330 relate to bispecific bivalent antibody formats.

PD-L1

Co-stimulation or the provision of two distinct signals to T-cells is a widely accepted model of lymphocyte activation of resting T lymphocytes by antigen-presenting cells (APCs) (Lafferty et al., Aust. J. Exp. Biol. Med. Sci. 53: 27-42 (1975)).

This model further provides for the discrimination of self from non-self and immune tolerance (Bretscher et al., Science 169: 1042-1049 (1970); Bretscher, P. A., P.N.A.S. USA 96: 185-190 (1999); Jenkins et al., J. Exp. Med. 165: 302-319 (1987)). The primary signal, or antigen specific signal, is transduced through the T-cell receptor (TCR) following recognition of foreign antigen peptide presented in the context of the major histocompatibility-complex (MHC). The second or co-stimulatory signal is delivered to T-cells by co-stimulatory molecules expressed on antigen-presenting cells (APCs), and induces T-cells to promote clonal expansion, cytokine secretion and effector function (Lenschow et al., Ann. Rev. Immunol. 14:233 (1996)). In the absence of co-stimulation, T-cells can become refractory to antigen stimulation, do not mount an effective immune response, and further may result in exhaustion or tolerance to foreign antigens.

The simple two-signal model can be an oversimplification because the strength of the TCR signal actually has a quantitative influence on T-cell activation and differentiation (Viola et al., Science 273: 104-106 (1996); Sloan-Lancaster, Nature 363: 156-159 (1993)). Moreover, T-cell activation can occur even in the absence of co-stimulatory signals if the TCR signal strength is high. More importantly, T-cells receive both positive and negative secondary co-stimulatory signals. The regulation of such positive and negative signals is critical to maximize the host's protective immune responses, while maintaining immune tolerance and preventing autoimmunity.

Negative secondary signals seem necessary for induction of T-cell tolerance, while positive signals promote T-cell activation. While the simple two-signal model still provides a valid explanation for naive lymphocytes, a host's immune response is a dynamic process, and co-stimulatory signals can also be provided to antigen-exposed T-cells.

The mechanism of co-stimulation is of therapeutic interest because the manipulation of co-stimulatory signals has shown to provide a means to either enhance or terminate cell-based immune response. Recently, it has been discovered that T cell dysfunction or anergy occurs concurrently with an induced and sustained expression of the inhibitory receptor, programmed death 1 polypeptide (PD-1). As a result, therapeutic targeting of PD-1 and other molecules which signal through interactions with PD-1, such as programmed death ligand 1 (PD-L1) and programmed death ligand 2 (PD-L2), are an area of intense interest. The inhibition of PD-L1 signaling has been proposed as a means to enhance T cell immunity for the treatment of cancer (e.g., tumor immunity) and infection, including both acute and chronic (e.g., persistent) infection. However, as an optimal therapeutic directed to a target in this pathway has yet to be commercialized, a significant unmet medical need exists.

Antibodies against PD-L1 are described e.g. in WO 2010/077634. WO 2010/077634 also suggests PD-L1 antibodies to be effective in the treatment of cancer. Further, said document suggests therapeutic use of said PD-L1 antibodies in a combination treatment of PD-L1 antibodies with traditional therapy, e.g. angiogenesis inhibition amongst others, to treat tumor immunity.

There is still a need for improved cancer therapies based on antibodies that bind to ANG-2, particularly on antibodies that bind to ANG-2 and VEGF.

### SUMMARY OF THE INVENTION

The inventors of the present invention have found that an antibody that binds to ANG2 and VEGF enhances the efficacy of an antibody that binds to PD-L1 in the treatment of cancers, in delaying progression of a tumor, or with respect to the survival of a patient afflicted with cancer, e.g. with a solid tumor. The delay of cancer progression, and the longer overall survival represent a major benefit for patients. Surprisingly, treatment of tumors with an antibody that binds to ANG-2 and VEGF in combination with an antibody that binds to PD-L1 showed a synergistic effect on the time of survival of the treated individuals.

The present invention relates to an antibody that binds to angiopoietin 2 (ANG-2), wherein the antibody is administered in a combination therapy with an antibody that binds to programmed death ligand 1 (PD-L1), for use in
a) treating cancer,
b) delaying progression of cancer,
c) prolonging the survival of a patient suffering from cancer, or
d) stimulating a cell mediated immune response.

In one embodiment, the antibody that binds to ANG-2 for use according to the invention is administered in combination therapy with an antibody that binds to vascular endothelial growth factor (VEGF) and with an antibody that binds to PD-L1.

In one embodiment, the antibody that binds to ANG-2 for use according to the invention binds to ANG-2 and to VEGF. In one embodiment, such antibody is a bispecific antibody.

In another aspect, the invention relates to an antibody that binds to PD-L1, wherein the antibody is administered in a combination therapy with an antibody that binds to ANG-2, for use in
a) treating cancer,
b) delaying progression of cancer,
c) prolonging the survival of a patient suffering from cancer, or
d) stimulating a cell mediated immune response.

In one embodiment, the antibody that binds to PD-L1 for use according to the invention is administered in a combination therapy with an antibody that binds to VEGF and with an antibody that binds to ANG-2. In one embodiment, the antibody that binds to PD-L1 for use according to the invention is administered in a combination therapy with an antibody that binds to ANG-2 and to VEGF. In one embodiment, such antibody binding to ANG-2 and VEGF is a bispecific antibody.

In a further aspect, the invention relates to an antibody that binds to VEGF, wherein the antibody is administered in a combination therapy with an antibody that binds to ANG-2 and with an antibody that binds to PD-L1, for use in
a) treating cancer,
b) delaying progression of cancer,
c) prolonging the survival of a patient suffering from cancer, or
d) stimulating a cell mediated immune response.

In one embodiment, the antibody that binds to VEGF for use according to the invention binds to VEGF and to ANG-2. In one embodiment, such antibody is a bispecific antibody.

In certain embodiments, the antibodies for use according to the invention are for use in
a) treating a solid tumor,
b) delaying progression of a solid tumor, or
c) prolonging the survival of a patient suffering from a solid tumor.

Further aspects of the invention relate to methods of treatment, uses of the antibodies for the manufacture of a medicament, pharmaceutical compositions comprising the antibodies, methods for the manufacture of a pharmaceutical composition comprising the antibodies, and articles of manufacture suitable for application in a combination therapy according to the invention.

### DESCRIPTION OF THE FIGURES

FIG. 1: Tumor growth inhibition upon treating test individuals with a combination therapy according to the invention (results of the experiment described in example 2). Indicated is the tumor growth inhibition (median+/−inter quartile range) until day 26 of the experiment.

FIG. 2: In vivo anti-tumor efficacy of a combination therapy according to the invention (results of the experiment described in example 2). Indicated is the overall survival of the treated groups.

### DETAILED DESCRIPTION OF THE INVENTION

### 1. Definitions

The terms “a”, “an” and “the” generally include plural referents, unless the context clearly indicates otherwise.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The terms “monoclonal antibody” or “monoclonal antibody composition” as used herein refer to a preparation of antibody molecules of a single amino acid composition.

A “recombinant antibody” is an antibody which has been produced by a recombinantly engineered host cell. It is optionally isolated or purified.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially the entire FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

“Specificity” refers to selective recognition of a particular epitope of an antigen by the antigen binding moiety, e.g. an antibody. Natural antibodies, for example, are monospecific. The term “monospecific antibody” as used herein denotes an antibody that has one or more binding sites each of which bind to the same epitope of the same antigen. “Multispecific antibodies” bind two or more different epitopes (for example, two, three, four, or more different epitopes). The epitopes may be on the same or different antigens. An example of a multispecific antibody is a “bispecific antibody” which binds two different epitopes. When an antibody possesses more than one specificity, the recognized epitopes may be associated with a single antigen or with more than one antigen.

An epitope is a region of an antigen that is bound by an antibody or antigen binding moiety. The term “epitope” includes any polypeptide determinant capable of specific binding to an antibody or antigen binding moiety. In certain embodiments, epitope determinants include chemically active surface groupings of molecules such as amino acids, glycan side chains, phosphoryl, or sulfonyl, and, in certain embodiments, may have specific three dimensional structural characteristics, and/or specific charge characteristics. Conformational and nonconformational epitopes are distinguished in that the binding to the former but not the latter is lost in the presence of denaturing solvents.

As used herein, the terms “binding” and “specific binding” refer to the binding of the antibody or antigen binding moiety to an epitope of the antigen in an in vitro assay, preferably in a plasmon resonance assay (BIAcore®, GE-Healthcare Uppsala, Sweden) with purified wild-type antigen.

The affinity of the binding of an antibody to an antigen is defined by the terms kₐ (rate constant for the association of the antibody from the antibody/antigen complex), k_{D} (dissociation constant), and K_{D} (k_{D}/ka). In one embodiment binding or that/which specifically binds to means a binding affinity (K_{D}) of 10⁻⁸ mol/l or less, in one embodiment 10⁻⁸ M to 10⁻¹³ mol/l. Thus, an multispecific antibody according to the invention specifically binds to each antigen for which it is specific with a binding affinity (K_{D}) of 10⁻⁸ mol/l or less, e.g. with a binding affinity (K_{D}) of 10⁻⁸ to 10⁻¹³ mol/l. in one embodiment with a binding affinity (K_{D}) of 10⁻⁹ to 10⁻¹³ mol/l.

The “variable domains” or “variable region” as used herein denotes each of the pair of light and heavy chains which is involved directly in binding the antibody to the antigen. The variable domain of a light chain is abbreviated as “VL” and the variable domain of a heavy chain is abbreviated as “VH”. The variable light and heavy chain domains have the same general structure and each domain comprises four framework (FR) regions whose sequences are widely conserved, connected by three “hypervariable regions” (or complementary determining regions, CDRs). The framework regions adopt a beta-sheet conformation and the CDRs may form loops connecting the beta-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies according to the invention and therefore provide a further object of the invention.

The term “antigen-binding portion of an antibody” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, Md. (1991) and/or those residues from a “hypervariable loop”.

The term “constant domains” or “constant region” as used within the current application denotes the sum of the domains of an antibody other than the variable region. The constant region is not directly involved in binding of an antigen, but exhibits various effector functions.

Depending on the amino acid sequence of the constant region of their heavy chains, antibodies are divided in the “classes”: IgA, IgD, IgE, IgG and IgM, and several of these may are further divided into subclasses, such as IgG1, IgG2, IgG3, and IgG4, IgA1 and IgA2. The heavy chain constant regions that correspond to the different classes of antibodies are called α, δ, ε, γ and μ, respectively. The light chain constant regions (CL) which can be found in all five antibody classes are called κ (kappa) and λ (lambda).

The “constant domains” as used in the antibodies disclosed herein are preferably from human origin, which is from a constant heavy chain region of a human antibody of the subclass IgG1, IgG2, IgG3, or IgG4 and/or a constant light chain kappa or lambda region. Such constant domains and regions are well known in the state of the art and e.g. described by Kabat, et al., Sequences of Proteins of Immunological Interest, 5th ed., Public Health Service, National Institutes of Health, Bethesda, Md. (1991).

The “Fc part” of an antibody is not involved directly in binding of an antibody to an antigen, but exhibit various effector functions. A “Fc part of an antibody” is a term well known to the skilled artisan and defined on the basis of papain cleavage of antibodies. Depending on the amino acid sequence of the constant region of their heavy chains, antibodies or immunoglobulins are divided in the classes: IgA, IgD, IgE, IgG and IgM, and several of these may be further divided into subclasses (isotypes), e.g. IgG1, IgG2, IgG3, and IgG4, IgA1, and IgA2. According to the heavy chain constant regions the different classes of immunoglobulins are called α, δ, ε, γ, and μ, respectively. The Fc part of an antibody is directly involved in ADCC (antibody-dependent cell-mediated cytotoxicity) and CDC (complement-dependent cytotoxicity) based on complement activation, C1q binding and Fc receptor binding. Complement activation (CDC) is initiated by binding of complement factor C1q to the Fc part of most IgG antibody subclasses. While the influence of an antibody on the complement system is dependent on certain conditions, binding to C1q is caused by defined binding sites in the Fc part. Such binding sites are known in the state of the art and described e.g. by Boackle, R. J., et al., Nature 282 (1979) 742-743; Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virology 75 (2001) 12161-12168; Morgan, A., et al., Immunology 86 (1995) 319-324; EP 0 307 434. Such binding sites are e.g. L234, L235, D270, N297, E318, K320, K322, P331 and P329 (numbering according to EU index of Kabat, E. A., see below). Antibodies of subclass IgG1, IgG2 and IgG3 usually show complement activation and C1q and C3 binding, whereas IgG4 do not activate the complement system and do not bind C1q and C3.

Human angiopoietin-2 (ANG-2) (alternatively abbreviated with ANGPT2 or ANG2) (SEQ ID NO: 1) is described in Maisonpierre, P. C., et al., Science 277 (1997) 55-60 and Cheung, A. H., et al, Genomics 48 (1998) 389-91. The angiopoietins-1 and -2 (ANG-1 and ANG-2) were discovered as ligands for the Ties, a family of tyrosine kinases that is selectively expressed within the vascular endothelium (Yancopoulos, G. D., et al., Nature 407 (2000) 242-48). There are now four definitive members of the angiopoietin family. Angiopoietin-3 and -4 (ANG-3 and ANG-4) may represent widely diverged counterparts of the same gene locus in mouse and man (Kim, I., et al., FEBS Let, 443 (1999) 353-56; Kim, I., et al., J Biol Chem 274 (1999) 26523-28). ANG-1 and ANG-2 were originally identified in tissue culture experiments as agonist and antagonist, respectively (see for ANG-1: Davies, S., et al., Cell, 87 (1996) 1161-1169; and for ANG-2: Maisonpierre, P. C., et al., Science 277 (1997) 55-60). All of the known angiopoietins bind primarily to Tie2, and both ANG-1 and ANG-2 bind to Tie2 with an affinity of 3 nM (Kd) (Maisonpierre, P. C., et al., Science 277 (1997) 55-60). ANG-1 was shown to support EC survival and to promote endothelium integrity (Davis, S., et al., Cell, 87 (1996) 1161-1169; Kwak, H. J., et al., FEBS Lett 448 (1999) 249-53; Suri, C., et al., Science 282 (1998) 468-71; Thurston, G., et al., Science 286 (1999) 2511-14; Thurston, G., et al., Nat. Med. 6 (2000) 460-63), whereas ANG-2 had the opposite effect and promoted blood vessel destabilization and regression in the absence of the survival factors VEGF or basic fibroblast growth factor (Maisonpierre, P. C., et al., Science 277 (1997) 55-60). However, many studies of ANG-2 function have suggested a more complex situation. ANG-2 might be a complex regulator of vascular remodeling that plays a role in both vessel sprouting and vessel regression. Supporting such roles for ANG-2, expression analyses reveal that ANG-2 is rapidly induced, together with VEGF, in adult settings of angiogenic sprouting, whereas ANG-2 is induced in the absence of VEGF in settings of vascular regression (Holash, J., et al., Science 284 (1999) 1994-98; Holash, J., et al., Oncogene 18 (1999) 5356-62). Consistent with a context-dependent role, ANG-2 specifically binds to the same endothelial-specific receptor, Tie-2, which is activated by ANG-1, but has context-dependent effects on its activation (Maisonpierre, P. C., et al., Science 277 (1997) 55-60).

Antibodies that bind to human ANG-2, which are useful for the combination treatment as described herein are, e.g., disclosed in detail in WO2010/069532 (e.g. antibodies <ANG-2>Ang2i_LC06, <ANG-2>Ang2i_LC07, or <ANG-2>Ang2i_LC10); WO2011/014469 (e.g. antibody H1 H685P); in US2011/150895 (e.g. antibodies SAIT-Ang-2-5, SAIT-Ang-2-6, or their humanized versions); in WO2009/097325 (e.g. antibody MEDI 1/5 characterized by the VL of SEQ ID NO:3 and the VH of SEQ ID NO:7, both SEQ ID NOs as numbered in WO2009/097325), in WO2009/105269; in WO 2006/068953 or in WO 03/030833. A particularly useful antibody that binds to human ANG-2 is antibody <ANG-2>Ang2i_LC06 as disclosed in WO2010/069532.

Bispecific antibodies that bind to human ANG-2 and to human VEGF, which are useful for the combination treatment as described herein are, e.g., disclosed in detail in WO2010/040508, WO 2011/117329 or WO2012/131078. Apart from bispecific antibodies specifically disclosed in the prior art, for the present invention, binding sites (e.g. VH and VL domains) of the anti-ANG-2 antibodies mentioned above may be included within a bispecific antibody that binds to human ANG-2 and human VEGF as used in an embodiment of the invention. In addition, the binding sites as disclosed in WO2010/040508, WO 2011/117329 or WO2012/131078 may be used.

When used herein, the term “angiopoietin 2” or “ANG-2” refers to the human protein angiopoietin 2 (SEQ ID NO: 1). As used herein, an antibody “binding to ANG-2”, “specifically binding to ANG-2”, “that binds to ANG-2” or “anti-ANG-2 antibody” refers to an antibody specifically binding to the human ANG-2 antigen with a binding affinity of a K_{D}-value of 1.0×10⁻⁸ mol/l or lower, in one embodiment of a K_{D}-value of 1.0×10⁻⁹ mol/l or lower, in one embodiment of a K_{D}-value of 1.0×10⁻⁹ mol/l to 1.0×10⁻¹³ mol/l. The binding affinity is determined with a standard binding assay, such as surface plasmon resonance technique (BIAcore®, GE-Healthcare Uppsala, Sweden).

When used herein, the term “VEGF” or “human VEGF” refers to the human protein VEGF (SEQ ID NO: 2). As used herein, an antibody “binding to VEGF”, “specifically binding to VEGF”, “that binds to VEGF” or “anti-VEGF antibody” refers to an antibody specifically binding to the human VEGF antigen with a binding affinity of a K_{D}-value of 1.0×10⁻⁸ mol/l or lower, in one embodiment of a K_{D}-value of 1.0×10⁻⁹ mol/l or lower, in one embodiment of a K_{D}-value of 1.0×10⁻⁹ mol/l to 1.0×10⁻¹³ mol/l. The binding affinity is determined with a standard binding assay, such as surface plasmon resonance technique (BIAcore®, GE-Healthcare Uppsala, Sweden).

When used herein, the term “PD-L1” or “human PD-L1” refers to the human protein programmed death 1 polypeptide (SEQ ID NO: 3). As used herein, an antibody “binding to PD-L1”, “specifically binding to PD-L1”, “that binds to PD-L1” or “anti-PD-L1 antibody” refers to an antibody specifically binding to the human PD-L1 antigen with a binding affinity of a K_{D}-value of 1.0×10⁻⁸ mol/l or lower, in one embodiment of a K_{D}-value of 1.0×10⁻⁹ mol/l or lower, in one embodiment of a K_{D}-value of 1.0×10⁻⁹ mol/l to 1.0×10⁻¹³ mol/l. The binding affinity is determined with a standard binding assay, such as surface plasmon resonance technique (BIAcore®, GE-Healthcare Uppsala, Sweden).

The terms “administered in combination with”, “co-administration”, “co-administering”, “combination therapy”, “administered with” or “combination treatment” refer to the administration of the anti-ANG2 antibody as described herein, and the anti-PD-L1 antibody as well as—in certain embodiments—the anti-VEGF antibody as described herein, e.g. as separate formulations/applications or as one single formulation/application. In one embodiment, the anti-ANG2 antibody and the anti-PD-L1 antibody are administered as separate formulations and in different application schemes. The co-administration can be simultaneous or sequential in either order, wherein preferably there is a time period while both (or all) active agents (i.e. the antibodies that binds to ANG-2 and PD-L1 and, in certain embodiments, the antibody that binds to VEGF) simultaneously exert their biological activities. Said anti-ANG2 antibody and said anti-PD-L1 antibody (and, in certain embodiments, the anti-VEGF antibody) are co-administered either simultaneously or sequentially (e.g. intravenous through a continuous infusion). When the therapeutic agents (i.e. the antibodies) are co-administered sequentially the dose is administered either on the same day in separate administrations, or one of the agents is administered on day 1 and the further agent(s) is/are co-administered within the next 7 days (preferably at day 2 to 4). Thus in one embodiment the term “sequentially” means within 7 days after the dose of the first component, preferably within 4 days after the dose of the first component; and the term “simultaneously” means at the same time. The terms “co-administration” with respect to the maintenance doses of anti-ANG2 antibody, and/or anti-PD-L1 antibody- and/or in certain embodiments the anti-VEGF antibody mean that the maintenance doses can be either co-administered simultaneously, if the treatment cycle is appropriate for all of the antibodies s, e.g. every week. Or the further agent(s) are e.g. administered e.g. every first to third day and said antibody is administered every week. Or the maintenance doses are co-administered sequentially, either within one or within several days.

The amount of co-administration and the timing of co-administration will depend on the type (species, gender, age, weight, etc.) and condition of the patient being treated and the severity of the disease or condition being treated. Said anti-ANG2 antibody and further agent are suitably co-administered to the patient at one time or over a series of treatments e.g. on the same day or on the day after.

It is self-evident that the antibodies are administered to the patient in a “therapeutically effective amount” (or simply “effective amount”) which is the amount of the respective compound or combination that will elicit the biological or medical response of a tissue, system, animal or human that is being sought by the researcher, veterinarian, medical doctor or other clinician.

Depending on the type and severity of the disease, about 0.1 mg/kg to 50 mg/kg (e.g. 0.1-20 mg/kg) of said anti-ANG2 antibody, anti-PD-L1 antibody and/or (in certain embodiments) anti-VEGF antibody is an initial candidate dosage for co-administration of said drugs to the patient.

As used herein, the term “patient”, “subject” or “individual” preferably refers to a human in need of treatment of cancer, or a precancerous condition or lesion. However, the term “patient”, “subject” or “individual” may also refer to non-human animals, e.g. mammals such as mice, dogs, cats, horses, cows, pigs, sheep and non-human primates, among others, that are in need of treatment.

The combination therapy disclosed herein may be co-administered with a chemotherapeutic agent. Such chemotherapeutic agents include, but are not limited to, anti-neoplastic agents including alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); Temodal™ (temozolamide), ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil (5FU), fluorodeoxyuridine, gemcitabine, cytosine arabinoside (AraC, cytarabine), 5-azacytidine, 2,2′-difluorodeoxycytidine, purine analogs such as 6-merca.rho.topurine, 6-thioguamne, azathioprine, T-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actinomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o, p-DDD) and aminoglutethimide; hormones and antagonists including adrenocorticosteroid antagonists such as prednisone and equivalents, dexamethasone and aminoglutethimide; Gemzar™ (gemcitabine), progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. In one embodiment the chemotherapeutic agent is selected from the group consisting of taxanes (like e.g. paclitaxel (Taxol), docetaxel (Taxotere), modified paclitaxel (e.g., Abraxane and Opaxio), doxorubicin, sunitinib (Sutent), sorafenib (Nexavar), and other multikinase inhibitors, oxaliplatin, cisplatin and carboplatin, etoposide, gemcitabine, and vinblastine. In one embodiment the chemotherapeutic agent is selected from the group consisting of taxanes (like e.g. taxol (paclitaxel), docetaxel (Taxotere), modified paclitaxel (e.g. Abraxane and Opaxio). In one embodiment, the additional chemotherapeutic agent is selected from 5-fluorouracil (5-FU), leucovorin, irinotecan, or oxaliplatin. In one embodiment the chemotherapeutic agent is 5-fluorouracil, leucovorin and irinotecan (FOLFIRI). In one embodiment the chemotherapeutic agent is 5-fluorouracil, and oxaliplatin (FOLFOX).

Specific examples of combination therapies with additional chemotherapeutic agents include, for instance, taxanes (e.g., docetaxel or paclitaxel), a modified paclitaxel (e.g., Abraxane or Opaxio), doxorubicin, capecitabine and/or bevacizumab (Avastin) for the treatment of breast cancer; therapies with carboplatin, oxaliplatin, cisplatin, paclitaxel, doxorubicin, modified doxorubicin (Caelyx or Doxil), or topotecan (Hycamtin) for ovarian cancer, the therapies with a multi-kinase inhibitor MKI (Sutent, Nexavar, or 706) and/or doxorubicin for treatment of kidney cancer; therapies with oxaliplatin, cisplatin and/or radiation for the treatment of squamous cell carcinoma; therapies with taxol and/or carboplatin for the treatment of lung cancer.

Therefore, in one embodiment the additional chemotherapeutic agent is selected from the group of taxanes (docetaxel or paclitaxel), a modified paclitaxel (Abraxane or Opaxio), doxorubicin, capecitabine and/or bevacizumab for the treatment of breast cancer.

The combination therapy disclosed herein may be co-administered with a therapeutic agent targeting epigenetic mechanism. Such agents include, but are not limited to, histone deacetylase inhibitors, demethylating agents (e.g., Vidaza) and release of transcriptional repression (ATRA) therapies can also be combined with the antigen binding proteins.

The combination therapy disclosed herein may be co-administered with a radiation therapy.

The antibodies used in a combination therapy as disclosed herein are preferably produced by recombinant methods. Such methods are widely known in the state of the art and comprise protein expression in prokaryotic and eukaryotic cells with subsequent isolation of the antibody polypeptide and usually purification to a pharmaceutically acceptable purity. For the protein expression nucleic acids encoding light and heavy chains or fragments thereof are inserted into expression vectors by standard methods. Expression is performed in appropriate prokaryotic or eukaryotic host cells, such as CHO cells, NS0 cells, SP2/0 cells, HEK293 cells, COS cells, yeast, or E. coli cells, and the antibody is recovered from the cells (from the supernatant or after cells lysis). Recombinant production of antibodies is well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; and Werner, R. G., Drug Res. 48 (1998) 870-880.

The antibodies may be present in whole cells, in a cell lysate, or in a partially purified, or substantially pure form. Purification is performed in order to eliminate other cellular components or other contaminants, e.g. other cellular nucleic acids or proteins, by standard techniques, including alkaline/SDS treatment, CsCl banding, column chromatography, agarose gel electrophoresis, and others well known in the art (see Ausubel, F., et al., ed. Current Protocols in Molecular Biology, Greene Publishing and Wiley Interscience, New York (1987)).

The heavy and light chain variable domains of the specific antibodies indicated herein that may be used in a combination therapy according to the invention are combined with sequences of promoter, translation initiation, constant region, 3′ untranslated region, polyadenylation, and transcription termination to form expression vector constructs. The heavy and light chain expression constructs can be combined into a single vector, co-transfected, serially transfected, or separately transfected into host cells which are then fused to form a single host cell expressing both chains. The control sequences that are suitable for prokaryotes, for example, include a promoter, optionally an operator sequence, and a ribosome binding site. Eukaryotic cells are known to utilize promoters, enhancers and polyadenylation signals.

The term “pharmaceutical composition” refers to a preparation which is in such form as to permit the biological activity of an active ingredient contained therein to be effective, and which contains no additional components which are unacceptably toxic to a subject to which the composition would be administered. A pharmaceutical composition of the present invention can be administered by a variety of methods known in the art. As will be appreciated by the skilled artisan, the route and/or mode of administration will vary depending upon the desired results.

Regardless of the route of administration selected, the antibodies used in a combination therapy of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient (i.e. the antibody) which is effective to achieve the desired therapeutic response for a particular patient, composition, and mode of administration, without being toxic to the patient (effective amount). The selected dosage level will depend upon a variety of pharmacokinetic factors including the activity of the particular compositions of the present invention employed, or the ester, salt or amide thereof, the route of administration, the time of administration, the rate of excretion of the particular compound being employed, other drugs, compounds and/or materials used in combination with the particular compositions employed, the age, sex, weight, condition, general health and prior medical history of the patient being treated, and like factors well known in the medical arts.

As used herein, “pharmaceutically acceptable carrier” includes any and all solvents, dispersion media, coatings, antibacterial and antifungal agents, isotonic and absorption/resorption delaying agents, and the like that are physiologically compatible. Preferably, the carrier is suitable for injection or infusion. Pharmaceutically acceptable carriers include sterile aqueous solutions or dispersions and sterile powders for the preparation of sterile injectable solutions or dispersion. The use of such media and agents for pharmaceutically active substances is known in the art. In addition to water, the carrier can be, for example, an isotonic buffered saline solution.

The pharmaceutical compositions according to the invention may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. Specific effects desired for the combination therapy of the present invention are specified below.

The term “a method of treating”, “method of treatment” or its equivalent, when applied to, for example, cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed to induce an overall beneficial course of action.

### 2. Detailed Description of the Embodiments of the Invention

The invention relates to a combination therapy, encompassing the co-administration of an antibody that binds to ANG-2 with an antibody that binds to PD-L1. The combination therapy is applicable in

a) treating cancer,

b) delaying progression of cancer,

c) prolonging the survival of a patient suffering from cancer, or

d) stimulating a cell mediated immune response, particularly stimulating cytotoxic T-lymphocytes, or stimulating macrophage activity. In one embodiment of the invention, the stimulation of the cell mediated immune response occurs during the treatment of cancer. In one embodiment, stimulating the cell mediated immune response as indicated under d) encompasses stimulating the activity of cytotoxic T-lymphocytes, or stimulating the activity of macrophages.

It has been shown that applying said combination therapy was potent to improve ANG-2 based cancer therapy in treated individuals suffering from cancer. The inventors of the present invention have found that anti-PD-L1 antibodies enhance the efficacy of anti-ANG-2 antibodies to treat cancers, delay progression of a tumor, or prolonging the survival of a patient afflicted with cancer e.g. with a solid tumor. The delay of cancer progression, as well as the longer overall survival represent a major benefit for patients.

The combination therapy of the invention may further encompass administration of an antibody that binds to VEGF. The aforementioned beneficial effects, particularly the delay of cancer progression and a longer overall survival of individuals suffering from cancer could be tremendously improved by using an anti-ANG-2 and an anti-VEGF antibody in the combination therapy indicated above.

Hence, in another aspect, the invention relates to a combination therapy, encompassing the co-administration of an antibody that binds to ANG-2, and an antibody that binds to VEGF with an antibody that binds to PD-L1. The combination therapy is applicable in

a) treating cancer,

b) delaying progression of cancer,

c) prolonging the survival of a patient suffering from cancer, or

d) stimulating a cell mediated immune response, particularly stimulating cytotoxic T-lymphocytes, or stimulating macrophage activity. In one embodiment of the invention, the stimulation of the cell mediated immune response occurs during the treatment of cancer. In one embodiment, stimulating the cell mediated immune response as indicated under d) encompasses stimulating the activity of cytotoxic T-lymphocytes, or stimulating the activity of macrophages. In one embodiment, said combination therapy encompasses the co-administration of an antibody that binds to ANG-2 and to VEGF (in one embodiment a bispecific antibody) with an antibody that binds to PD-L1.

It has been shown that applying said combination therapy was potent to improve cancer therapy based on administration of anti-ANG-2/VEGF antibodies in treated individuals suffering from cancer. The inventors of the present invention have found that anti-PD-L1 antibodies enhance the efficacy of anti-ANG2/VEGF antibodies antibodies to treat cancers, delay progression of a tumor, or prolonging the survival of a patient afflicted with cancer e.g. with a solid tumor. The delay of cancer progression, as well as the longer overall survival represent a major benefit for patients.

### Antibody for Use

One aspect of the invention relates to an antibody that binds to ANG-2, or an antibody that binds to PD-L1 for use in a combination therapy according to the invention.

Another aspect of the invention relates to an antibody that binds to ANG-2, an antibody that binds to VEGF or an antibody that binds to PD-L1 for use in a combination therapy according to the invention. In certain embodiments of this aspect of the invention the combination therapy according to the invention encompasses co-administration of an antibody that binds to ANG-2 and to VEGF (in one embodiment a bispecific antibody) with an antibody that binds to PD-L1.

### Anti-ANG-2 Antibody for Use

In one embodiment the invention relates to an antibody that binds to angiopoietin 2 (ANG-2), wherein the antibody is administered in a combination therapy with an antibody that binds to PD-L1, for use in

a) treating cancer,

b) delaying progression of cancer,

c) prolonging the survival of a patient suffering from cancer, or

d) stimulating a cell mediated immune response.

In one embodiment the invention relates to an antibody that binds to ANG-2, wherein the antibody is administered in a combination therapy with an antibody that binds to PD-L1, for use in

a) treating cancer,

b) delaying progression of cancer, or

c) prolonging the survival of a patient suffering from cancer.

In one embodiment the invention relates to an antibody that binds to ANG-2, wherein the antibody is administered in a combination therapy with an antibody that binds to PD-L1, for use in treating cancer. In one embodiment the invention relates to an antibody that binds to ANG-2, wherein the antibody is administered in a combination therapy with an antibody that binds to PD-L1, for use in prolonging the survival of a patient suffering from cancer.

In one embodiment the invention relates to an antibody that binds to ANG-2, wherein the antibody is administered in combination therapy with an antibody that binds to VEGF and with an antibody that binds to PD-L1, for use in

a) treating cancer,

b) delaying progression of cancer,

c) prolonging the survival of a patient suffering from cancer, or

d) stimulating a cell mediated immune response.

According to this embodiment of the invention, the antibody that binds to ANG-2 and the antibody that binds to VEGF may be provided as separate antibodies or may be provided in a form of a single antibody that binds to ANG-2 as well as to VEGF.

In one embodiment the invention relates to an antibody that binds to ANG-2, wherein the antibody is administered in combination therapy with an antibody that binds to VEGF and with an antibody that binds to PD-L1, for use in

a) treating cancer,

b) delaying progression of cancer, or

c) prolonging the survival of a patient suffering from cancer.

In one embodiment the invention relates to an antibody that binds to ANG-2, wherein the antibody is administered in combination with an antibody that binds to VEGF, and wherein the antibody is administered in combination with an antibody that binds to PD-L1, for use in treating cancer. In one embodiment the invention relates to an antibody that binds to ANG-2, wherein the antibody is administered in combination with an antibody that binds to VEGF, and wherein the antibody is administered in combination with an antibody that binds to PD-L1, for use in prolonging the survival of a patient suffering from cancer.

In one embodiment the invention relates to an antibody that binds to ANG-2 and VEGF, wherein the antibody is administered in a combination therapy with an antibody that binds to PD-L1, for use in

a) treating cancer,

b) delaying progression of cancer,

c) prolonging the survival of a patient suffering from cancer; or

d) stimulating a cell mediated immune response.

According to this embodiment of the invention, the antibody that binds to ANG-2 and the antibody that binds to VEGF is provided in a form of a single antibody that binds to ANG-2 as well as to VEGF. In one embodiment, said antibody that binds to ANG-2 and VEGF is a bispecific antibody.

In one embodiment the invention relates to an antibody, in one embodiment a bispecific antibody, that binds to ANG-2 and VEGF, wherein the antibody is administered in a combination therapy with an antibody that binds to PD-L1, for use in

a) treating cancer,

b) delaying progression of cancer, or

c) prolonging the survival of a patient suffering from cancer.

In one embodiment the invention relates to an antibody, in one embodiment a bispecific antibody, that binds to ANG-2 and VEGF, wherein the antibody is administered in a combination therapy with an antibody that binds to PD-L1, for use in treating cancer. In one embodiment the invention relates to an antibody, in one embodiment a bispecific antibody, that binds to ANG-2 and VEGF, wherein the antibody is administered in a combination therapy with an antibody that binds to PD-L1, for use in prolonging the survival of a patient suffering from cancer.

### Anti-PD-L1 Antibody for Use

In one embodiment the invention relates to an antibody that binds to PD-L1, wherein the antibody is administered in a combination therapy with an antibody that binds to ANG-2, for use in

a) treating cancer,

b) delaying progression of cancer,

c) prolonging the survival of a patient suffering from cancer, or

d) stimulating a cell mediated immune response.

In one embodiment the invention relates to an antibody that binds to PD-L1, wherein the antibody is administered in a combination therapy with an antibody that binds to ANG-2, for use in

a) treating cancer,

b) delaying progression of cancer, or

c) prolonging the survival of a patient suffering from cancer.

In one embodiment the invention relates to an antibody that binds to PD-L1, wherein the antibody is administered in a combination therapy with an antibody that binds to ANG-2, for use in treating cancer. In one embodiment the invention relates to an antibody that binds to PD-L1, wherein the antibody is administered in a combination therapy with an antibody that binds to ANG-2, for use in prolonging the survival of a patient suffering from cancer.

In one embodiment the invention relates to an antibody that binds to PD-L1, wherein the antibody is administered in a combination therapy with an antibody that binds to ANG-2 and an with antibody that binds to VEGF, for use in

a) treating cancer,

b) delaying progression of cancer,

c) prolonging the survival of a patient suffering from cancer, or

d) stimulating a cell mediated immune response.

According to this embodiment of the invention, the antibody that binds to ANG-2 and the antibody that binds to VEGF may be provided as separate antibodies or may be provided in a form of a single antibody that binds to ANG-2 as well as to VEGF.

In one embodiment the invention relates to an antibody that binds to PD-L1, wherein the antibody is administered in combination therapy with an antibody that binds to ANG-2 and with an antibody that binds to VEGF, for use in

a) treating cancer,

b) delaying progression of cancer, or

c) prolonging the survival of a patient suffering from cancer.

In one embodiment the invention relates to an antibody that binds to PD-L1, wherein the antibody is administered in combination therapy with an antibody that binds to ANG-2 and with an antibody that binds to VEGF, for use in treating cancer. In one embodiment the invention relates to an antibody that binds to PD-L1, wherein the antibody is administered in combination therapy with an antibody that binds to ANG-2 and with an antibody that binds to VEGF, for use in prolonging the survival of a patient suffering from cancer.

In one embodiment the invention relates to an antibody that binds to PD-L1, wherein the antibody is administered in a combination therapy with an antibody that binds to ANG-2 and VEGF, for use in

a) treating cancer,

b) delaying progression of cancer,

c) prolonging the survival of a patient suffering from cancer, or

d) stimulating a cell mediated immune response.

According to this embodiment of the invention, the antibody that binds to ANG-2 and the antibody that binds to VEGF is provided in a form of a single antibody that binds to ANG-2 as well as to VEGF. In one embodiment, said antibody that binds to ANG-2 and VEGF is a bispecific antibody.

In one embodiment the invention relates to an antibody that binds to PD-L1, wherein the antibody is administered in a combination therapy with an antibody, in one embodiment a bispecific antibody, that binds to ANG-2 and VEGF, for use in

a) treating cancer,

b) delaying progression of cancer, or

c) prolonging the survival of a patient suffering from cancer.

In one embodiment the invention relates to an antibody that binds to PD-L1, wherein the antibody is administered in a combination therapy with an antibody, in one embodiment a bispecific antibody, that binds to ANG-2 and VEGF, for use in treating cancer. In one embodiment the invention relates to an antibody that binds to PD-L1, wherein the antibody is administered in a combination therapy with an antibody, in one embodiment a bispecific antibody, that binds to ANG-2 and VEGF, for use in prolonging the survival of a patient suffering from cancer.

### Anti-VEGF Antibody for Use

One aspect of the invention further encompasses the co-administration of an antibody that binds to VEGF in addition to the co-adminstration of an antibody that binds to ANG-2 and an antibody that binds to PD-L1.

In one embodiment the invention relates to an antibody that binds to VEGF, wherein the antibody is administered in a combination therapy with an antibody that binds to ANG-2 and with an antibody that binds to PD-L1, for use in

a) treating cancer,

b) delaying progression of cancer,

c) prolonging the survival of a patient suffering from cancer, or

d) stimulating a cell mediated immune response.

According to this embodiment of the invention, the antibody that binds to ANG-2 and the antibody that binds to VEGF may be provided as separate antibodies or may be provided in a form of a single antibody that binds to ANG-2 as well as to VEGF.

In one embodiment the invention relates to an antibody that binds to VEGF, wherein the antibody is administered in combination therapy with an antibody that binds to ANG-2 and with an antibody that binds to PD-L1, for use in

a) treating cancer,

b) delaying progression of cancer, or

c) prolonging the survival of a patient suffering from cancer.

In one embodiment the invention relates to an antibody that binds to VEGF, wherein the antibody is administered in combination therapy with an antibody that binds to ANG-2 and with an antibody that binds to PD-L1, for use in treating cancer. In one embodiment the invention relates to an antibody that binds to VEGF, wherein the antibody is administered in combination therapy with an antibody that binds to ANG-2 and with an antibody that binds to PD-L1, for use in prolonging the survival of a patient suffering from cancer.

### Methods of Treatment

In one embodiment the invention relates to a method for

a) treating cancer,

b) delaying progression of cancer,

c) prolonging the survival of a patient suffering from cancer, or

d) stimulating a cell mediated immune response,

wherein the method comprises the step of administering an effective amount of an antibody that binds to ANG-2 and an effective amount of an antibody that binds to PD-L1 to a patient in need thereof.

In one embodiment the invention relates to a method for

a) treating cancer,

b) delaying progression of cancer, or

c) prolonging the survival of a patient suffering from cancer,

wherein the method comprises the step of administering an effective amount of an antibody that binds to ANG-2 and an effective amount of an antibody that binds to PD-L1 to a patient in need thereof.

In one embodiment the invention relates to a method for treating cancer, wherein the method comprises the step of administering an effective amount of an antibody that binds to ANG-2 and an effective amount of an antibody that binds to PD-L1 to a patient in need thereof. In one embodiment the invention relates to a method for prolonging the survival of a patient suffering from cancer, wherein the method comprises the step of administering an effective amount of an antibody that binds to ANG-2 and an effective amount of an antibody that binds to PD-L1 to a patient in need thereof.

In one embodiment the invention relates to a method for

a) treating cancer,

b) delaying progression of cancer,

c) prolonging the survival of a patient suffering from cancer, or

d) stimulating a cell mediated immune response,

wherein the method comprises the step of administering an effective amount of an antibody that binds to ANG-2, an effective amount of an antibody that binds to VEGF; and an effective amount of an antibody that binds to PD-L1 to a patient in need thereof.

According to this embodiment of the invention, the antibody that binds to ANG-2 and the antibody that binds to VEGF may be provided as separate antibodies or may be provided in a form of a single antibody that binds to ANG-2 as well as to VEGF.

In one embodiment the invention relates to a method for

a) treating cancer,

b) delaying progression of cancer, or

c) prolonging the survival of a patient suffering from cancer,

wherein the method comprises the step of administering an effective amount of an antibody that binds to ANG-2, an effective amount of an antibody that binds to VEGF; and an effective amount of an antibody that binds to PD-L1 to a patient in need thereof.

In one embodiment the invention relates to a method for treating cancer, wherein the method comprises the step of administering an effective amount of an antibody that binds to ANG-2, an effective amount of an antibody that binds to VEGF; and an effective amount of an antibody that binds to PD-L1 to a patient in need thereof. In one embodiment the invention relates to a method for prolonging the survival of a patient suffering from cancer, wherein the method comprises the step of administering an effective amount of an antibody that binds to ANG-2, an effective amount of an antibody that binds to VEGF; and an effective amount of an antibody that binds to PD-L1 to a patient in need thereof.

In one embodiment the invention relates to a method for

a) treating cancer,

b) delaying progression of cancer,

c) prolonging the survival of a patient suffering from cancer, or

d) stimulating a cell mediated immune response,

wherein the method comprises the step of administering an effective amount of an antibody that binds to ANG-2 and to VEGF; and an effective amount of an antibody that binds to PD-L1 to a patient in need thereof.

According to this embodiment of the invention, the antibody that binds to ANG-2 and the antibody that binds to VEGF is provided in a form of a single antibody that binds to ANG-2 as well as to VEGF. In one embodiment, said antibody that binds to ANG-2 and VEGF is a bispecific antibody.

In one embodiment the invention relates to a method for

a) treating cancer,

b) delaying progression of cancer, or

c) prolonging the survival of a patient suffering from cancer,

wherein the method comprises the step of administering an effective amount of an antibody, in one embodiment a bispecific antibody, that binds to ANG-2 and to VEGF; and an effective amount of an antibody that binds to PD-L1 to a patient in need thereof.

In one embodiment the invention relates to a method for treating cancer, wherein the method comprises the step of administering an effective amount of an antibody, in one embodiment a bispecific antibody, that binds to ANG-2 and to VEGF; and an effective amount of an antibody that binds to PD-L1 to a patient in need thereof. In one embodiment the invention relates to a method for prolonging the survival of a patient suffering from cancer, wherein the method comprises the step of administering an effective amount of an antibody, in one embodiment a bispecific antibody, that binds to ANG-2 and to VEGF; and an effective amount of an antibody that binds to PD-L1 to a patient in need thereof.

### Use of Antibody for Manufacture of a Medicament

One aspect of the invention relates to an antibody that binds to ANG-2, or an antibody that binds to PD-L1 for use in the manufacture of a medicament for the use in a combination therapy according to the invention.

Another aspect of the invention relates to an antibody that binds to ANG-2, an antibody that binds to VEGF, or an antibody that binds to PD-L1 for use in the manufacture of a medicament for the use in a combination therapy according to the invention. In certain embodiments of this aspect of the invention an antibody that binds to ANG-2 and to VEGF (in one embodiment a bispecific antibody) is applied for use in the manufacture of the medicament for the use in a combination therapy according to the invention.

### Use of Anti-ANG-2 Antibody

In one embodiment the invention relates to the use of an antibody that binds to ANG-2 for the manufacture of a medicament for

a) treating cancer,

b) delaying progression of cancer,

c) prolonging the survival of a patient suffering from cancer, or

d) stimulating a cell mediated immune response,

wherein the antibody is for administration in a combination therapy with an antibody that binds to PD-L1.

In one embodiment the invention relates to the use of an antibody that binds to ANG-2 for the manufacture of a medicament for

a) treating cancer,

b) delaying progression of cancer, or

c) prolonging the survival of a patient suffering from cancer,

wherein the antibody is for administration in a combination therapy with an antibody that binds to PD-L1.

In one embodiment the invention relates to the use of an antibody that binds to ANG-2 for the manufacture of a medicament for treating cancer, wherein the antibody is for administration in a combination therapy with an antibody that binds to PD-L1. In one embodiment the invention relates to the use of an antibody that binds to ANG-2 for the manufacture of a medicament for prolonging the survival of a patient suffering from cancer, wherein the antibody is for administration in a combination therapy with an antibody that binds to PD-L1.

In one embodiment the invention relates to the use of an antibody that binds to ANG-2 for the manufacture of a medicament for

a) treating cancer,

b) delaying progression of cancer,

c) prolonging the survival of a patient suffering from cancer, or

d) stimulating a cell mediated immune response,

wherein the antibody is for administration in a combination therapy with an antibody that binds to VEGF and an antibody that binds to PD-L1.

According to this embodiment of the invention, the antibody that binds to ANG-2 and the antibody that binds to VEGF may be provided as separate antibodies or may be provided in a form of a single antibody that binds to ANG-2 as well as to VEGF.

In one embodiment the invention relates to the use of an antibody that binds to ANG-2 for the manufacture of a medicament for

a) treating cancer,

b) delaying progression of cancer, or

c) prolonging the survival of a patient suffering from cancer,

wherein the antibody is for administration in a combination therapy with an antibody that binds to VEGF and an antibody that binds to PD-L1.

In one embodiment the invention relates to the use of an antibody that binds to ANG-2 for the manufacture of a medicament for treating cancer, wherein the antibody is for administration in a combination therapy with an antibody that binds to VEGF and an antibody that binds to PD-L1. In one embodiment the invention relates to the use of an antibody that binds to ANG-2 for the manufacture of a medicament for prolonging the survival of a patient suffering from cancer, wherein the antibody is for administration in a combination therapy with an antibody that binds to VEGF and an antibody that binds to PD-L1.

In one embodiment the invention relates to the use of an antibody that binds to ANG-2 and VEGF for the manufacture of a medicament for

a) treating cancer,

b) delaying progression of cancer,

c) prolonging the survival of a patient suffering from cancer, or

d) stimulating a cell mediated immune response,

wherein the antibody is for administration in a combination therapy with an antibody that binds to VEGF and an antibody that binds to PD-L1.

According to this embodiment of the invention, the antibody that binds to ANG-2 and the antibody that binds to VEGF is provided in a form of a single antibody that binds to ANG-2 as well as to VEGF. In one embodiment, said antibody that binds to ANG-2 and VEGF is a bispecific antibody.

In one embodiment the invention relates to the use of an antibody that binds to ANG-2 and VEGF, in one embodiment a bispecific antibody, for the manufacture of a medicament for

a) treating cancer,

b) delaying progression of cancer, or

c) prolonging the survival of a patient suffering from cancer,

wherein the antibody is for administration in a combination therapy with an antibody that binds to PD-L1.

In one embodiment the invention relates to the use of an antibody that binds to ANG-2 and VEGF, in one embodiment a bispecific antibody, for the manufacture of a medicament for treating cancer, wherein the antibody is for administration in a combination therapy with an antibody that binds to PD-L1.

In one embodiment the invention relates to the use of an antibody that binds to ANG-2 and VEGF, in one embodiment a bispecific antibody, for the manufacture of a medicament for prolonging the survival of a patient suffering from cancer, wherein the antibody is for administration in a combination therapy with an antibody that binds to PD-L1.

### Use of Anti-PD-L1 Antibody

In one embodiment the invention relates to the use of an antibody that binds to PD-L1 for the manufacture of a medicament for

a) treating cancer,

b) delaying progression of cancer,

c) prolonging the survival of a patient suffering from cancer, or

d) stimulating a cell mediated immune response,

wherein the antibody is for administration in a combination therapy with an antibody that binds to ANG-2.

In one embodiment the invention relates to the use of an antibody that binds to PD-L1 for the manufacture of a medicament for

a) treating cancer,

b) delaying progression of cancer, or

c) prolonging the survival of a patient suffering from cancer,

wherein the antibody is for administration in a combination therapy with an antibody that binds to ANG-2.

In one embodiment the invention relates to the use of an antibody that binds to PD-L1 for the manufacture of a medicament for treating cancer, wherein the antibody is for administration in a combination therapy with an antibody that binds to ANG-2. In one embodiment the invention relates to the use of an antibody that binds to PD-L1 for the manufacture of a medicament for prolonging the survival of a patient suffering from cancer, wherein the antibody is for administration in a combination therapy with an antibody that binds to ANG-2.

In one embodiment the invention relates to the use of an antibody that binds to PD-L1 for the manufacture of a medicament for

a) treating cancer,

b) delaying progression of cancer,

c) prolonging the survival of a patient suffering from cancer, or

d) stimulating a cell mediated immune response,

wherein the antibody is for administration in a combination therapy with an antibody that binds to ANG-2 and an antibody that binds to VEGF.

According to this embodiment of the invention, the antibody that binds to ANG-2 and the antibody that binds to VEGF may be provided as separate antibodies or may be provided in a form of a single antibody that binds to ANG-2 as well as to VEGF.

In one embodiment the invention relates to the use of an antibody that binds to PD-L1 for the manufacture of a medicament for

a) treating cancer,

b) delaying progression of cancer, or

c) prolonging the survival of a patient suffering from cancer,

wherein the antibody is for administration in a combination therapy with an antibody that binds to ANG-2 and an antibody that binds to VEGF.

In one embodiment the invention relates to the use of an antibody that binds to PD-L1 for the manufacture of a medicament for treating cancer, wherein the antibody is for administration in a combination therapy with an antibody that binds to ANG-2 and an antibody that binds to VEGF. In one embodiment the invention relates to the use of an antibody that binds to PD-L1 for the manufacture of a medicament for prolonging the survival of a patient suffering from cancer, wherein the antibody is for administration in a combination therapy with an antibody that binds to ANG-2 and an antibody that binds to VEGF.

In one embodiment the invention relates to the use of an antibody that binds to PD-L1 for the manufacture of a medicament for

a) treating cancer,

b) delaying progression of cancer,

c) prolonging the survival of a patient suffering from cancer, or

d) stimulating a cell mediated immune response,

wherein the antibody is for administration in a combination therapy with an antibody that binds to ANG-2 and VEGF.

According to this embodiment of the invention, the antibody that binds to ANG-2 and the antibody that binds to VEGF is provided in a form of a single antibody that binds to ANG-2 as well as to VEGF. In one embodiment, said antibody that binds to ANG-2 and VEGF is a bispecific antibody.

In one embodiment the invention relates to the use of an antibody that binds to PD-L1 for the manufacture of a medicament for

a) treating cancer,

b) delaying progression of cancer, or

c) prolonging the survival of a patient suffering from cancer,

wherein the antibody is for administration in a combination therapy with an antibody, in one embodiment a bispecific antibody, that binds to ANG-2 and VEGF.

In one embodiment the invention relates to the use of an antibody that binds to PD-L1 for the manufacture of a medicament for treating cancer, wherein the antibody is for administration in a combination therapy with an antibody, in one embodiment a bispecific antibody, that binds to ANG-2 and VEGF.

In one embodiment the invention relates to the use of an antibody that binds to PD-L1 for the manufacture of a medicament for prolonging the survival of a patient suffering from cancer, wherein the antibody is for administration in a combination therapy with an antibody, in one embodiment a bispecific antibody, that binds to ANG-2 and VEGF.

### Use of Anti-VEGF Antibody

One embodiment of the invention further encompasses the co-administration of an antibody that binds to VEGF in addition to the co-adminstration of an antibody that binds to ANG-2 and an antibody that binds to PD-L1.

Hence, in one embodiment the invention relates to the use of an antibody that binds to VEGF for the manufacture of a medicament for

a) treating cancer,

b) delaying progression of cancer,

c) prolonging the survival of a patient suffering from cancer, or

d) stimulating a cell mediated immune response,

wherein the antibody is for administration in a combination therapy with an antibody that binds to ANG-2 and an antibody that binds to PD-L1.

According to this embodiment of the invention, the antibody that binds to ANG-2 and the antibody that binds to VEGF may be provided as separate antibodies or may be provided in a form of a single antibody that binds to ANG-2 as well as to VEGF.

In one embodiment the invention relates to the use of an antibody that binds to VEGF for the manufacture of a medicament for

a) treating cancer,

b) delaying progression of cancer, or

c) prolonging the survival of a patient suffering from cancer,

wherein the antibody is for administration in a combination therapy with an antibody that binds to ANG-2 and an antibody that binds to PD-L1.

In one embodiment the invention relates to the use of an antibody that binds to VEGF for the manufacture of a medicament for treating cancer, wherein the antibody is for administration in a combination therapy with an antibody that binds to ANG-2 and an antibody that binds to PD-L1 In one embodiment the invention relates to the use of an antibody that binds to VEGF for the manufacture of a medicament for prolonging the survival of a patient suffering from cancer, wherein the antibody is for administration in a combination therapy with an antibody that binds to ANG-2 and an antibody that binds to PD-L1.

### Use of an Anti-ANG-2 Antibody and an Anti-PD-L1 Antibody

In one embodiment the invention relates to the use of an antibody that binds to ANG-2 and an antibody that binds to PD-L1 for the manufacture of a medicament for

a) treating cancer,

b) delaying progression of cancer,

c) prolonging the survival of a patient suffering from cancer, or

d) stimulating a cell mediated immune response.

In one embodiment the invention relates to the use of an antibody that binds to ANG-2 and an antibody that binds to PD-L1 for the manufacture of a medicament for

a) treating cancer,

b) delaying progression of cancer, or

c) prolonging the survival of a patient suffering from cancer.

In one embodiment the invention relates to the use of an antibody that binds to ANG-2 and an antibody that binds to PD-L1 for the manufacture of a medicament for treating cancer, wherein the antibody is for administration in a combination therapy with an antibody that binds to PD-L1. In one embodiment the invention relates to the use of an antibody that binds to ANG-2 and an antibody that binds to PD-L1 for the manufacture of a medicament for prolonging the survival of a patient suffering from cancer, wherein the antibody is for administration in a combination therapy with an antibody that binds to PD-L1.

Use of an Anti-ANG-2 Antibody and an Anti-VEGF Antibody

In one embodiment the invention relates to the use of an antibody that binds to ANG-2 and an antibody that binds to VEGF for the manufacture of a medicament for

a) treating cancer,

b) delaying progression of cancer,

c) prolonging the survival of a patient suffering from cancer, or

d) stimulating a cell mediated immune response,

wherein the antibodies are for administration in a combination therapy with an antibody that binds to PD-L1.

In one embodiment the invention relates to the use of an antibody that binds to ANG-2 and an antibody that binds to VEGF for the manufacture of a medicament for

a) treating cancer,

b) delaying progression of cancer, or

c) prolonging the survival of a patient suffering from cancer,

wherein the antibodies are for administration in a combination therapy with an antibody that binds to PD-L1.

In one embodiment the invention relates to the use of an antibody that binds to ANG-2 and an antibody that binds to VEGF for the manufacture of a medicament for treating cancer, wherein the antibodies are for administration in a combination therapy with an antibody that binds to PD-L1. In one embodiment the invention relates to the use of an antibody that binds to ANG-2 and an antibody that binds to VEGF for the manufacture of a medicament for prolonging the survival of a patient suffering from cancer, wherein the antibodies are for administration in a combination therapy with an antibody that binds to PD-L1.

### Use of an Anti-ANG-2 Antibody, an Anti-VEGF Antibody and an Anti-PD-L1 Antibody

In one embodiment the invention relates to the use of an antibody that binds to ANG-2, an antibody that binds to VEGF, and an antibody that binds to PD-L1 for the manufacture of a medicament for

a) treating cancer,

b) delaying progression of cancer,

c) prolonging the survival of a patient suffering from cancer, or

d) stimulating a cell mediated immune response.

According to this embodiment of the invention, the antibody that binds to ANG-2 and the antibody that binds to VEGF may be provided as separate antibodies or may be provided in a form of a single antibody that binds to ANG-2 as well as to VEGF.

In one embodiment the invention relates to the use of an antibody that binds to ANG-2, an antibody that binds to VEGF, and an antibody that binds to PD-L1 for the manufacture of a medicament for

a) treating cancer,

b) delaying progression of cancer, or

c) prolonging the survival of a patient suffering from cancer.

In one embodiment the invention relates to the use of an antibody that binds to ANG-2, an antibody that binds to VEGF, and an antibody that binds to PD-L1 for the manufacture of a medicament for treating cancer. In one embodiment the invention relates to the use of an antibody that binds to ANG-2, an antibody that binds to VEGF, and an antibody that binds to PD-L1 for the manufacture of a medicament for prolonging the survival of a patient suffering from cancer.

In one embodiment the invention relates to the use of an antibody that binds to ANG-2 and VEGF, and an antibody that binds to PD-L1 for the manufacture of a medicament for

a) treating cancer,

b) delaying progression of cancer,

c) prolonging the survival of a patient suffering from cancer, or

d) stimulating a cell mediated immune response

According to this embodiment of the invention, the antibody that binds to ANG-2 and the antibody that binds to VEGF is provided in a form of a single antibody that binds to ANG-2 as well as to VEGF. In one embodiment, said antibody that binds to ANG-2 and VEGF is a bispecific antibody.

In one embodiment the invention relates to the use of an antibody that binds to ANG-2 and VEGF, and an antibody that binds to PD-L1 for the manufacture of a medicament for

a) treating cancer,

b) delaying progression of cancer, or

c) prolonging the survival of a patient suffering from cancer.

In one embodiment the invention relates to the use of an antibody that binds to ANG-2 and VEGF, and an antibody that binds to PD-L1 for the manufacture of a medicament for treating cancer. In one embodiment the invention relates to the use of an antibody that binds to ANG-2 and VEGF, and an antibody that binds to PD-L1 for the manufacture of a medicament for prolonging the survival of a patient suffering from cancer.

### Article of Manufacture

One aspect of the invention relates to an article of manufacture comprising a container, a composition within the container comprising an antibody that binds to ANG-2, or an antibody that binds to PD-L1, and a package insert instructing the user of the composition to administer the respective antibody in a a combination therapy according to the invention.

Another aspect of the invention relates to an article of manufacture comprising a container, a composition within the container comprising an antibody that binds to ANG-2, an antibody that binds to VEGF or an antibody that binds to PD-L1, and a package insert instructing the user of the composition to administer the respective antibody in a a combination therapy according to the invention. In certain embodiments of this aspect of the invention an antibody that binds to ANG-2 and to VEGF (in one embodiment a bispecific antibody) is comprises in the article of manufacture according to the invention.

Article of Manufacture comprising an Anti-ANG-2 Antibody

In one embodiment the invention relates to an article of manufacture comprising (a) a container, a composition within the container comprising an antibody that binds to ANG-2, and (b) a package insert instructing the user of the composition to administer the antibody that binds to ANG-2 to a patient

a) in the treatment of cancer,

b) to delay progression of cancer,

c) to prolong the survival of a patient suffering from cancer, or

d) to stimulate a cell mediated immune response,

wherein the administration of the antibody that binds to ANG-2 is in a combination therapy with an antibody that binds to PD-L1.

In one embodiment the invention relates to an article of manufacture comprising (a) a container, a composition within the container comprising an antibody that binds to ANG-2, and (b) a package insert instructing the user of the composition to administer the antibody that binds to ANG-2 to a patient

a) in the treatment of cancer,

b) to delay progression of cancer, or

c) to prolong the survival of a patient suffering from cancer,

wherein the administration of the antibody that binds to ANG-2 is in a combination therapy with an antibody that binds to PD-L1.

In one embodiment the invention relates to an article of manufacture comprising (a) a container, a composition within the container comprising an antibody that binds to ANG-2, and (b) a package insert instructing the user of the composition to administer the antibody that binds to ANG-2 to a patient in the treatment of cancer, wherein the administration of the antibody that binds to ANG-2 is in a combination therapy with an antibody that binds to PD-L1. In one embodiment the invention relates to an article of manufacture comprising (a) a container, a composition within the container comprising an antibody that binds to ANG-2, and (b) a package insert instructing the user of the composition to administer the antibody that binds to ANG-2 to a patient to prolong the survival of a patient suffering from cancer, wherein the administration of the antibody that binds to ANG-2 is in a combination therapy with an antibody that binds to PD-L1.

In one embodiment the invention relates to an article of manufacture comprising (a) a container, a composition within the container comprising an antibody that binds to ANG-2, and (b) a package insert instructing the user of the composition to administer the antibody that binds to ANG-2 to a patient

a) in the treatment of cancer,

b) to delay progression of cancer,

c) to prolong the survival of a patient suffering from cancer, or

d) to stimulate a cell mediated immune response,

wherein the administration of the antibody that binds to ANG-2 is in a combination therapy with an antibody that binds to VEGF and an antibody that binds to PD-L1.

In one embodiment the invention relates to an article of manufacture comprising (a) a container, a composition within the container comprising an antibody that binds to ANG-2, and (b) a package insert instructing the user of the composition to administer the antibody that binds to ANG-2 to a patient

a) in the treatment of cancer,

b) to delay progression of cancer, or

c) to prolong the survival of a patient suffering from cancer,

wherein the administration of the antibody that binds to ANG-2 is in a combination therapy with an antibody that binds to VEGF and an antibody that binds to PD-L1.

In one embodiment the invention relates to an article of manufacture comprising (a) a container, a composition within the container comprising an antibody that binds to ANG-2, and (b) a package insert instructing the user of the composition to administer the antibody that binds to ANG-2 to a patient in the treatment of cancer, wherein the administration of the antibody that binds to ANG-2 is in a combination therapy with an antibody that binds to VEGF and an antibody that binds to PD-L1. In one embodiment the invention relates to an article of manufacture comprising (a) a container, a composition within the container comprising an antibody that binds to ANG-2, and (b) a package insert instructing the user of the composition to administer the antibody that binds to ANG-2 to a patient to prolong the survival of a patient suffering from cancer, wherein the administration of the antibody that binds to ANG-2 is in a combination therapy with an antibody that binds to VEGF and an antibody that binds to PD-L1.

Article of Manufacture comprising an Anti-PD-L1 Antibody

In one embodiment the invention relates to an article of manufacture comprising (a) a container, a composition within the container comprising an antibody that binds to PD-L1, and (b) a package insert instructing the user of the composition to administer the antibody that binds to PD-L1 to a patient

a) in the treatment of cancer,

b) to delay progression of cancer,

c) to prolong the survival of a patient suffering from cancer, or

d) to stimulate a cell mediated immune response,

wherein the administration of the antibody that binds to PD-L1 is in a combination therapy with an antibody that binds to ANG-2.

In one embodiment the invention relates to an article of manufacture comprising (a) a container, a composition within the container comprising an antibody that binds to PD-L1, and (b) a package insert instructing the user of the composition to administer the antibody that binds to PD-L1 to a patient

a) in the treatment of cancer,

b) to delay progression of cancer, or

c) to prolong the survival of a patient suffering from cancer,

wherein the administration of the antibody that binds to PD-L1 is in a combination therapy with an antibody that binds to ANG-2.

In one embodiment the invention relates to an article of manufacture comprising (a) a container, a composition within the container comprising an antibody that binds to PD-L1, and (b) a package insert instructing the user of the composition to administer the antibody that binds to PD-L1 to a patient in the treatment of cancer, wherein the administration of the antibody that binds to PD-L1 is in a combination therapy with an antibody that binds to ANG-2. In one embodiment the invention relates to an article of manufacture comprising (a) a container, a composition within the container comprising an antibody that binds to PD-L1, and (b) a package insert instructing the user of the composition to administer the antibody that binds to PD-L1 to a patient to prolong the survival of a patient suffering from cancer, wherein the administration of the antibody that binds to PD-L1 is in a combination therapy with an antibody that binds to ANG-2.

In one embodiment the invention relates to an article of manufacture comprising (a) a container, a composition within the container comprising an antibody that binds to PD-L1, and (b) a package insert instructing the user of the composition to administer the antibody that binds to PD-L1 to a patient

a) in the treatment of cancer,

b) to delay progression of cancer,

c) to prolong the survival of a patient suffering from cancer, or

d) to stimulate a cell mediated immune response,

wherein the administration of the antibody that binds to PD-L1 is in a combination therapy with an antibody that binds to ANG-2 and an antibody that binds to VEGF.

In one embodiment the invention relates to an article of manufacture comprising (a) a container, a composition within the container comprising an antibody that binds to PD-L1, and (b) a package insert instructing the user of the composition to administer the antibody that binds to PD-L1 to a patient

a) in the treatment of cancer,

b) to delay progression of cancer, or

c) to prolong the survival of a patient suffering from cancer,

wherein the administration of the antibody that binds to PD-L1 is in a combination therapy with an antibody that binds to ANG-2 and an antibody that binds to VEGF.

In one embodiment the invention relates to an article of manufacture comprising (a) a container, a composition within the container comprising an antibody that binds to PD-L1, and (b) a package insert instructing the user of the composition to administer the antibody that binds to PD-L1 to a patient in the treatment of cancer, wherein the administration of the antibody that binds to PD-L1 is in a combination therapy with an antibody that binds to ANG-2 and an antibody that binds to VEGF. In one embodiment the invention relates to an article of manufacture comprising (a) a container, a composition within the container comprising an antibody that binds to PD-L1, and (b) a package insert instructing the user of the composition to administer the antibody that binds to PD-L1 to a patient to prolong the survival of a patient suffering from cancer, wherein the administration of the antibody that binds to PD-L1 is in a combination therapy with an antibody that binds to ANG-2 and an antibody that binds to VEGF.

Article of Manufacture comprising an Anti-VEGF Antibody

One embodiment of the invention further encompasses the co-administration of an antibody that binds to VEGF in addition to the co-adminstration of an antibody that binds to ANG-2 and an antibody that binds to PD-L1.

In one embodiment the invention relates to an article of manufacture comprising (a) a container, a composition within the container comprising an antibody that binds to VEGF, and (b) a package insert instructing the user of the composition to administer the antibody that binds to VEGF to a patient

a) in the treatment of cancer,

b) to delay progression of cancer,

c) to prolong the survival of a patient suffering from cancer, or

d) to stimulate a cell mediated immune response,

wherein the administration of the antibody that binds to VEGF is in a combination therapy with an antibody that binds to ANG-2 and an antibody that binds to PD-L1.

In one embodiment the invention relates to an article of manufacture comprising (a) a container, a composition within the container comprising an antibody that binds to VEGF, and (b) a package insert instructing the user of the composition to administer the antibody that binds to VEGF to a patient

a) in the treatment of cancer,

b) to delay progression of cancer, or

c) to prolong the survival of a patient suffering from cancer,

wherein the administration of the antibody that binds to VEGF is in a combination therapy with an antibody that binds to ANG-2 and an antibody that binds to PD-L1.

In one embodiment the invention relates to an article of manufacture comprising (a) a container, a composition within the container comprising an antibody that binds to VEGF, and (b) a package insert instructing the user of the composition to administer the antibody that binds to VEGF to a patient in the treatment of cancer, wherein the administration of the antibody that binds to VEGF is in a combination therapy with an antibody that binds to ANG-2 and an antibody that binds to PD-L1. In one embodiment the invention relates to an article of manufacture comprising (a) a container, a composition within the container comprising an antibody that binds to VEGF, and (b) a package insert instructing the user of the composition to administer the antibody that binds to VEGF to a patient to prolong the survival of a patient suffering from cancer, wherein the administration of the antibody that binds to VEGF is in a combination therapy with an antibody that binds to ANG-2 and an antibody that binds to PD-L1.

Article of Manufacture comprising an Anti-ANG-2 Antibody and an Anti-PD-L1 Antibody

In one embodiment the invention relates to an article of manufacture comprising (a) a container, a composition within the container comprising an antibody that binds to ANG-2, and (b) a container, a composition within the container comprising an antibody that binds to PD-L1, and (c) a package insert instructing the user of the composition to administer the antibody that binds to ANG-2 and the antibody that binds to PD-L1 to a patient

a) in the treatment of cancer,

b) to delay progression of cancer,

c) to prolong the survival of a patient suffering from cancer, or

d) to stimulate a cell mediated immune response.

In one embodiment the invention relates to an article of manufacture comprising (a) a container, a composition within the container comprising an antibody that binds to ANG-2, and (b) a container, a composition within the container comprising an antibody that binds to VEGF, and (c) a package insert instructing the user of the composition to administer the antibody that binds to ANG-2 and the antibody that binds to VEGF to a patient

a) in the treatment of cancer,

b) to delay progression of cancer, or

c) to prolong the survival of a patient suffering from cancer.

In one embodiment the invention relates to an article of manufacture comprising (a) a container, a composition within the container comprising an antibody that binds to ANG-2, and (b) a container, a composition within the container comprising an antibody that binds to VEGF, and (c) a package insert instructing the user of the composition to administer the antibody that binds to ANG-2 and the antibody that binds to VEGF to a patient in the treatment of cancer. In one embodiment the invention relates to an article of manufacture comprising (a) a container, a composition within the container comprising an antibody that binds to ANG-2, and (b) a container, a composition within the container comprising an antibody that binds to VEGF, and (c) a package insert instructing the user of the composition to administer the antibody that binds to ANG-2 and the antibody that binds to VEGF to a patient to prolong the survival of a patient suffering from cancer.

In one embodiment the invention relates to an article of manufacture comprising (a) a container, a composition within the container comprising an antibody that binds to ANG-2, and (b) a container, a composition within the container comprising an antibody that binds to PD-L1, and (c) a package insert instructing the user of the composition to administer the antibody that binds to ANG-2 and the antibody that binds to PD-L1 to a patient

a) in the treatment of cancer,

b) to delay progression of cancer,

c) to prolong the survival of a patient suffering from cancer, or

d) to stimulate a cell mediated immune response,

wherein the administration of the antibody that binds to ANG-2 and the antibody that binds to PD-L1 is in a combination therapy with an antibody that binds to VEGF.

In one embodiment the invention relates to an article of manufacture comprising (a) a container, a composition within the container comprising an antibody that binds to ANG-2, and (b) a container, a composition within the container comprising an antibody that binds to VEGF, and (c) a package insert instructing the user of the composition to administer the antibody that binds to ANG-2 and the antibody that binds to VEGF to a patient

a) in the treatment of cancer,

b) to delay progression of cancer, or

c) to prolong the survival of a patient suffering from cancer,

wherein the administration of the antibody that binds to ANG-2 and the antibody that binds to PD-L1 is in a combination therapy with an antibody that binds to VEGF.

In one embodiment the invention relates to an article of manufacture comprising (a) a container, a composition within the container comprising an antibody that binds to ANG-2, and (b) a container, a composition within the container comprising an antibody that binds to VEGF, and (c) a package insert instructing the user of the composition to administer the antibody that binds to ANG-2 and the antibody that binds to VEGF to a patient in the treatment of cancer, wherein the administration of the antibody that binds to ANG-2 and the antibody that binds to PD-L1 is in a combination therapy with an antibody that binds to VEGF. In one embodiment the invention relates to an article of manufacture comprising (a) a container, a composition within the container comprising an antibody that binds to ANG-2, and (b) a container, a composition within the container comprising an antibody that binds to VEGF, and (c) a package insert instructing the user of the composition to administer the antibody that binds to ANG-2 and the antibody that binds to VEGF to a patient to prolong the survival of a patient suffering from cancer, wherein the administration of the antibody that binds to ANG-2 and the antibody that binds to PD-L1 is in a combination therapy with an antibody that binds to VEGF.

Article of Manufacture comprising an Anti-ANG-2 Antibody and an Anti-VEGF Antibody

In one embodiment the invention relates to an article of manufacture comprising (a) a container, a composition within the container comprising an antibody that binds to ANG-2, and (b) a container, a composition within the container comprising an antibody that binds to VEGF, and (c) a package insert instructing the user of the composition to administer the antibody that binds to ANG-2 and the antibody that binds to VEGF to a patient

a) in the treatment of cancer,

b) to delay progression of cancer,

c) to prolong the survival of a patient suffering from cancer, or

d) to stimulate a cell mediated immune response,

wherein the administration of the antibody that binds to ANG-2 and the antibody that binds to VEGF is in a combination therapy with an antibody that binds to PD-L1.

In one embodiment of said article of manufacture, the article of manufacture comprises a container, a composition within said container comprising the antibody that binds to ANG-2 and the antibody that binds to VEGF. In one embodiment of said article of manufacture, said container comprises the antibody that binds to ANG-2 and the antibody that binds to VEGF in the form of an antibody that binds to ANG-2 and to VEGF. In one embodiment of said article of manufacture, said container comprises the antibody that binds to ANG-2 and the antibody that binds to VEGF in the form of a bispecific antibody that binds to ANG-2 and to VEGF.

In one embodiment the invention relates to an article of manufacture comprising (a) a container, a composition within the container comprising an antibody that binds to ANG-2, and (b) a container, a composition within the container comprising an antibody that binds to VEGF (in one embodiment the article of manufacture comprises one container, a composition within said container comprising the antibody that binds to ANG-2 and the antibody that binds to VEGF; in another embodiment the article of manufacture comprises one container, a composition within said container comprising the antibody that binds to ANG-2 and the antibody that binds to VEGF in the form of an antibody that binds to ANG-2 and to VEGF; in another embodiment the article of manufacture comprises one container, a composition within said container comprising the antibody that binds to ANG-2 and the antibody that binds to VEGF in the form of a bispecific antibody that binds to ANG-2 and to VEGF), and (c) a package insert instructing the user of the composition to administer the antibody that binds to ANG-2 and the antibody that binds to VEGF to a patient

a) in the treatment of cancer,

b) to delay progression of cancer, or

c) to prolong the survival of a patient suffering from cancer,

wherein the administration of the antibody that binds to ANG-2 and the antibody that binds to VEGF is in a combination therapy with an antibody that binds to PD-L1.

In one embodiment the invention relates to an article of manufacture comprising (a) a container, a composition within the container comprising an antibody that binds to ANG-2, and (b) a container, a composition within the container comprising an antibody that binds to VEGF (in one embodiment the article of manufacture comprises one container, a composition within said container comprising the antibody that binds to ANG-2 and the antibody that binds to VEGF; in another embodiment the article of manufacture comprises one container, a composition within said container comprising the antibody that binds to ANG-2 and the antibody that binds to VEGF in the form of an antibody that binds to ANG-2 and to VEGF; in another embodiment the article of manufacture comprises one container, a composition within said container comprising the antibody that binds to ANG-2 and the antibody that binds to VEGF in the form of a bispecific antibody that binds to ANG-2 and to VEGF), and (c) a package insert instructing the user of the composition to administer the antibody that binds to ANG-2 and the antibody that binds to VEGF to a patient in the treatment of cancer, wherein the administration of the antibody that binds to ANG-2 and the antibody that binds to VEGF is in a combination therapy with an antibody that binds to PD-L1.

In one embodiment the invention relates to an article of manufacture comprising (a) a container, a composition within the container comprising an antibody that binds to ANG-2, and (b) a container, a composition within the container comprising an antibody that binds to VEGF (in one embodiment the article of manufacture comprises one container, a composition within said container comprising the antibody that binds to ANG-2 and the antibody that binds to VEGF; in another embodiment the article of manufacture comprises one container, a composition within said container comprising the antibody that binds to ANG-2 and the antibody that binds to VEGF in the form of an antibody that binds to ANG-2 and to VEGF; in another embodiment the article of manufacture comprises one container, a composition within said container comprising the antibody that binds to ANG-2 and the antibody that binds to VEGF in the form of a bispecific antibody that binds to ANG-2 and to VEGF), and (c) a package insert instructing the user of the composition to administer the antibody that binds to ANG-2 and the antibody that binds to VEGF to a patient to prolong the survival of a patient suffering from cancer, wherein the administration of the antibody that binds to ANG-2 and the antibody that binds to VEGF is in a combination therapy with an antibody that binds to human PD-L1.

Article of Manufacture comprising an Anti-ANG-2 Antibody, an Anti-VEGF Antibody and an Anti-PD-L1 Antibody

In one embodiment the invention relates to an article of manufacture comprising (a) a container, a composition within the container comprising an antibody that binds to ANG-2, and (b) a container, a composition within the container comprising an antibody that binds to VEGF, and (c) a container, a composition within the container comprising an antibody that binds to PD-L1, and (d) a package insert instructing the user of the composition to administer the antibodies that bind to ANG-2, VEGF, and PD-L1, respectively, in a combination therapy to a patient

a) in the treatment of cancer,

b) to delay progression of cancer,

c) to prolong the survival of a patient suffering from cancer, or

d) to stimulate a cell mediated immune response.

In one embodiment of said article of manufacture, the article of manufacture comprises a container, a composition within said container comprising the antibody that binds to ANG-2 and the antibody that binds to VEGF. In one embodiment of said article of manufacture, said container comprises the antibody that binds to ANG-2 and the antibody that binds to VEGF in the form of an antibody that binds to ANG-2 and to VEGF. In one embodiment of said article of manufacture, said container comprises the antibody that binds to ANG-2 and the antibody that binds to VEGF in the form of a bispecific antibody that binds to ANG-2 and to VEGF.

In one embodiment the invention relates to an article of manufacture comprising (a) a container, a composition within the container comprising an antibody that binds to ANG-2, and (b) container, a composition within the container comprising an antibody that binds to VEGF (in one embodiment the article of manufacture comprises one container, a composition within said container comprising the antibody that binds to ANG-2 and the antibody that binds to VEGF; in another embodiment the article of manufacture comprises one container, a composition within said container comprising the antibody that binds to ANG-2 and the antibody that binds to VEGF in the form of an antibody that binds to ANG-2 and to VEGF; in another embodiment the article of manufacture comprises one container, a composition within said container comprising the antibody that binds to ANG-2 and the antibody that binds to VEGF in the form of a bispecific antibody that binds to ANG-2 and to VEGF), and (c) a container, a composition within the container comprising an antibody that binds to PD-L1, and (d) a package insert instructing the user of the composition to administer the antibodies that bind to ANG-2, VEGF, and PD-L1, respectively, in a combination therapy to a patient

a) in the treatment of cancer,

b) to delay progression of cancer, or

c) to prolong the survival of a patient suffering from cancer.

In one embodiment the invention relates to an article of manufacture comprising (a) a container, a composition within the container comprising an antibody that binds to ANG-2, and (b) container, a composition within the container comprising an antibody that binds to VEGF (in one embodiment the article of manufacture comprises one container, a composition within said container comprising the antibody that binds to ANG-2 and the antibody that binds to VEGF; in another embodiment the article of manufacture comprises one container, a composition within said container comprising the antibody that binds to ANG-2 and the antibody that binds to VEGF in the form of an antibody that binds to ANG-2 and to VEGF; in another embodiment the article of manufacture comprises one container, a composition within said container comprising the antibody that binds to ANG-2 and the antibody that binds to VEGF in the form of a bispecific antibody that binds to ANG-2 and to VEGF), and (c) a container, a composition within the container comprising an antibody that binds to PD-L1, and (d) a package insert instructing the user of the composition to administer the antibodies that bind to ANG-2, VEGF, and PD-L1, respectively, in a combination therapy to a patient in the treatment of cancer.

In one embodiment the invention relates to an article of manufacture comprising (a) a container, a composition within the container comprising an antibody that binds to ANG-2, and (b) container, a composition within the container comprising an antibody that binds to VEGF (in one embodiment the article of manufacture comprises one container, a composition within said container comprising the antibody that binds to ANG-2 and the antibody that binds to VEGF; in another embodiment the article of manufacture comprises one container, a composition within said container comprising the antibody that binds to ANG-2 and the antibody that binds to VEGF in the form of an antibody that binds to ANG-2 and to VEGF; in another embodiment the article of manufacture comprises one container, a composition within said container comprising the antibody that binds to ANG-2 and the antibody that binds to VEGF in the form of a bispecific antibody that binds to ANG-2 and to VEGF), and (c) a container, a composition within the container comprising an antibody that binds to PD-L1, and (d) a package insert instructing the user of the composition to administer the antibodies that bind to ANG-2, VEGF, and PD-L1, respectively, in a combination therapy to a patient to prolong the survival of a patient suffering from cancer.

### Pharmaceutical Composition

In one embodiment the invention relates to a pharmaceutical composition, comprising an antibody that binds to ANG-2, and an antibody that binds to PD-L1, wherein each antibody is formulated together with a pharmaceutically acceptable carrier.

In one embodiment of said pharmaceutical composition, the antibody that binds to ANG-2 and the antibody that binds to PD-L1 are formulated separately. In another embodiment of said pharmaceutical composition, the antibody that binds to ANG-2 and the antibody that binds to PD-L1 are formulated together.

In one embodiment the invention relates to a pharmaceutical composition, comprising an antibody that binds to ANG-2, an antibody that binds to VEGF, and an antibody that binds to PD-L1, wherein each antibody is formulated together with a pharmaceutically acceptable carrier.

In one embodiment of said pharmaceutical composition comprising an antibody that binds to ANG-2, an antibody that binds to VEGF, and an antibody that binds to PD-L1, the antibody that binds to ANG-2 and the antibody that binds to VEGF are comprised in the form of one antibody that binds to ANG-2 and to VEGF. In one embodiment of said pharmaceutical composition, the antibody that binds to ANG-2 and to VEGF is bispecific.

In one embodiment of said pharmaceutical composition comprising an antibody that binds to ANG-2 and VEGF, and an antibody that binds to PD-L1, the antibody that binds to ANG-2 and VEGF, and the antibody that binds to PD-L1 are formulated separately. In another embodiment of said pharmaceutical composition comprising an antibody that binds to ANG-2 and VEGF, and an antibody that binds to PD-L1, the antibody that binds to ANG-2 and VEGF, and the antibody that binds to PD-L1 are formulated together.

In another embodiment of said pharmaceutical composition comprising an antibody that binds to ANG-2, an antibody that binds to VEGF, and an antibody that binds to PD-L1, the antibody that binds to ANG-2, the antibody that binds to VEGF, and the antibody that binds to PD-L1 are formulated separately.

In another embodiment of said pharmaceutical composition comprising an antibody that binds to ANG-2, an antibody that binds to VEGF, and an antibody that binds to PD-L1, the antibody that binds to ANG-2, the antibody that binds to VEGF, and the antibody that binds to PD-L1 are formulated together.

In another embodiment of said pharmaceutical composition comprising an antibody that binds to ANG-2 and VEGF, and an antibody that binds to PD-L1, the antibody that binds to ANG-2 and VEGF, and the antibody that binds to PD-L1 are formulated separately.

### Method for the Manufacture of a Pharmaceutical Composition

In one embodiment the invention relates to a method for the manufacture of a pharmaceutical composition, comprising (a) formulating an antibody that binds to ANG-2 with a pharmaceutically acceptable carrier, and (b) formulating separately an antibody that binds to PD-L1 with a pharmaceutically acceptable carrier.

In one embodiment the invention relates to a method for the manufacture of a pharmaceutical composition, comprising formulating an antibody that binds to ANG-2 together with an antibody that binds to PD-L1 in combination with a pharmaceutically acceptable carrier.

In one embodiment the invention relates to a method for the manufacture of a pharmaceutical composition, comprising (a) formulating an antibody that binds to ANG-2 with a pharmaceutically acceptable carrier, (b) formulating separately an antibody that binds to VEGF with a pharmaceutically acceptable carrier and (c) formulating separately an antibody that binds to PD-L1 with a pharmaceutically acceptable carrier.

In one embodiment the invention relates to a method for the manufacture of a pharmaceutical composition, comprising (a) formulating an antibody that binds to ANG-2 together with an antibody that binds to VEGF in combination with a pharmaceutically acceptable carrier, and (b) formulating separately an antibody that binds to PD-L1 with a pharmaceutically acceptable carrier.

In one embodiment the invention relates to a method for the manufacture of a pharmaceutical composition, comprising formulating an antibody that binds to ANG-2 together with an antibody that binds to VEGF and together with an antibody that binds to PD-L1 in combination with a pharmaceutically acceptable carrier.

In one embodiment the invention relates to a method for the manufacture of a pharmaceutical composition, comprising (a) formulating an antibody that binds to ANG-2 and VEGF with a pharmaceutically acceptable carrier, and (b) formulating separately an antibody that binds to PD-L1 with a pharmaceutically acceptable carrier.

In one embodiment the invention relates to a method for the manufacture of a pharmaceutical composition, comprising formulating an antibody that binds to ANG-2 and VEGF together with an antibody that binds to PD-L1 in combination with a pharmaceutically acceptable carrier.

### Specific Antibodies

In one embodiment of the invention, the antibody that binds to ANG-2 that is used in a combination therapy according to the invention binds to human ANG-2.

In one embodiment of the invention, the antibody that binds to ANG-2 that is used in a combination therapy according to the invention comprises the following variable domain amino acid sequences: variable heavy chain domain VH of SEQ ID NO: 4, and variable light chain domain VL of SEQ ID NO: 5 (corresponding to the VH and VL domains of <ANG-2> LC06 as disclosed herein).

In one embodiment of the invention, the antibody that binds to ANG-2 that is used in a combination therapy according to the invention comprises the following variable domain amino acid sequences: variable heavy chain domain VH of SEQ ID NO: 10, and variable light chain domain VL of SEQ ID NO: 5 (corresponding to the VH and VL domains of <ANG-2> E6Q as disclosed herein).

In one embodiment of the invention, the antibody that binds to PD-L1 that is used in a combination therapy according to the invention binds to human PD-L1.

In one embodiment of the invention, the antibody that binds to PD-L1 that is used in a combination therapy according to the invention comprises variable domain amino acid sequences, selected from the group of:
variable heavy chain domain VH of SEQ ID NO: 17, and variable light chain domain VL of SEQ ID NO: 20 (corresponding to the VH and VL domains of <PD-L1> “243.55.S70” as disclosed herein);
variable heavy chain domain VH of SEQ ID NO: 18, and variable light chain domain VL of SEQ ID NO: 21 (corresponding to the VH and VL domains of <PD-L1> “243.55.H1” as disclosed herein);
variable heavy chain domain VH of SEQ ID NO: 18, and variable light chain domain VL of SEQ ID NO: 22 (corresponding to the VH and VL domains of <PD-L1> “243.55.H12” as disclosed herein);
variable heavy chain domain VH of SEQ ID NO: 18, and variable light chain domain VL of SEQ ID NO: 23 (corresponding to the VH and VL domains of <PD-L1> “243.55.H37” as disclosed herein);
variable heavy chain domain VH of SEQ ID NO: 18, and variable light chain domain VL of SEQ ID NO: 24 (corresponding to the VH and VL domains of <PD-L1> “243.55.H70” as disclosed herein);
variable heavy chain domain VH of SEQ ID NO: 18, and variable light chain domain VL of SEQ ID NO: 25 (corresponding to the VH and VL domains of <PD-L1> “243.55.H89” as disclosed herein);
variable heavy chain domain VH of SEQ ID NO: 18, and variable light chain domain VL of SEQ ID NO: 26 (corresponding to the VH and VL domains of <PD-L1> “243.55.S1” as disclosed herein);
variable heavy chain domain VH of SEQ ID NO: 18, and variable light chain domain VL of SEQ ID NO: 27 (corresponding to the VH and VL domains of <PD-L1> “243.55.5” as disclosed herein);
variable heavy chain domain VH of SEQ ID NO: 18, and variable light chain domain VL of SEQ ID NO: 28 (corresponding to the VH and VL domains of <PD-L1> “243.55.8” as disclosed herein);
variable heavy chain domain VH of SEQ ID NO: 18, and variable light chain domain VL of SEQ ID NO: 29 (corresponding to the VH and VL domains of <PD-L1> “243.55.30” as disclosed herein);
variable heavy chain domain VH of SEQ ID NO: 18, and variable light chain domain VL of SEQ ID NO: 30 (corresponding to the VH and VL domains of <PD-L1> “243.55.34” as disclosed herein);
variable heavy chain domain VH of SEQ ID NO: 18, and variable light chain domain VL of SEQ ID NO: 31 (corresponding to the VH and VL domains of <PD-L1> “243.55.S37” as disclosed herein);
variable heavy chain domain VH of SEQ ID NO: 18, and variable light chain domain VL of SEQ ID NO: 32 (corresponding to the VH and VL domains of <PD-L1> “243.55.49” as disclosed herein);
variable heavy chain domain VH of SEQ ID NO: 18, and variable light chain domain VL of SEQ ID NO: 33 (corresponding to the VH and VL domains of <PD-L1> “243.55.51” as disclosed herein);
variable heavy chain domain VH of SEQ ID NO: 18, and variable light chain domain VL of SEQ ID NO: 34 (corresponding to the VH and VL domains of <PD-L1> “243.55.62” as disclosed herein); and
variable heavy chain domain VH of SEQ ID NO: 19, and variable light chain domain VL of SEQ ID NO: 35 (corresponding to the VH and VL domains of <PD-L1> “243.55.84” as disclosed herein).

In one embodiment of the invention, the antibody that binds to PD-L1 that is used in a combination therapy according to the invention comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70” as disclosed herein).

In one embodiment of the invention, the antibody that binds to VEGF that is used in a combination therapy according to the invention binds to human VEGF.

In one embodiment of the invention, the antibody that binds to VEGF that is used in a combination therapy according to certain embodiments of the invention comprises the following variable domain amino acid sequences: variable heavy chain domain VH of SEQ ID NO: 8, and variable light chain domain VL of SEQ ID NO: 9 (corresponding to the VH and VL domains of <VEGF> bevacizumab as disclosed herein).

In one embodiment of the invention, the antibody that binds to VEGF that is used in a combination therapy according to certain embodiments of the invention comprises the following variable domain amino acid sequences: variable heavy chain domain VH of SEQ ID NO: 6, and variable light chain domain VL of SEQ ID NO: 7 (corresponding to the VH and VL domains of <VEGF> B20.4.1 as disclosed herein).

In one embodiment of the invention, the combination therapy of the present invention uses the antibody that binds to ANG-2 comprising the variable heavy chain domain VH of SEQ ID NO:4, and the variable light chain domain VL of SEQ ID NO: 5 (corresponding to the VH and VL domains of <ANG-2> LC06 as disclosed herein); and the antibody that binds to VEGF comprising the variable heavy chain domain VH of SEQ ID NO: 8, and the variable light chain domain VL of SEQ ID NO: 9 (corresponding to the VH and VL domains of <VEGF> bevacizumab as disclosed herein).

In one embodiment of the invention, the combination therapy of the present invention uses the antibody that binds to ANG-2 comprising the variable heavy chain domain VH of SEQ ID NO: 10, and the variable light chain domain VL of SEQ ID NO: 5 (corresponding to the VH and VL domains of <ANG-2> E6Q as disclosed herein); and the antibody that binds to VEGF comprising the variable heavy chain domain VH of SEQ ID NO: 8, and the variable light chain domain VL of SEQ ID NO: 9 (corresponding to the VH and VL domains of <VEGF> bevacizumab as disclosed herein).

In one embodiment of the invention, the combination therapy of the present invention uses the antibody that binds to ANG-2 comprising the variable heavy chain domain VH of SEQ ID NO:4, and the variable light chain domain VL of SEQ ID NO: 5 (corresponding to the VH and VL domains of <ANG-2> LC06 as disclosed herein); and the antibody that binds to VEGF comprising the variable heavy chain domain VH of SEQ ID NO: 6, and the variable light chain domain VL of SEQ ID NO: 7 (corresponding to the VH and VL domains of <VEGF> B20.4.1 as disclosed herein).

In one embodiment of the invention, the combination therapy of the present invention uses the antibody that binds to ANG-2 comprising the variable heavy chain domain VH of SEQ ID NO: 10, and the variable light chain domain VL of SEQ ID NO: 5 (corresponding to the VH and VL domains of <ANG-2> E6Q as disclosed herein); and the antibody that binds to VEGF comprising the variable heavy chain domain VH of SEQ ID NO: 6, and the variable light chain domain VL of SEQ ID NO: 7 (corresponding to the VH and VL domains of <VEGF> B20.4.1 as disclosed herein).

In one embodiment of the invention, the combination therapy of the present invention uses the antibody that binds to ANG-2 comprising the variable heavy chain domain VH of SEQ ID NO:4, and the variable light chain domain VL of SEQ ID NO: 5 (corresponding to the VH and VL domains of <ANG-2> LC06 as disclosed herein); and an antibody that binds to PD-L1 comprising variable domain amino acid sequences, selected from the group of:
variable heavy chain domain VH of SEQ ID NO: 17, and variable light chain domain VL of SEQ ID NO: 20 (corresponding to the VH and VL domains of <PD-L1> “243.55.S70” as disclosed herein);
variable heavy chain domain VH of SEQ ID NO: 18, and variable light chain domain VL of SEQ ID NO: 21 (corresponding to the VH and VL domains of <PD-L1> “243.55.H1” as disclosed herein);
variable heavy chain domain VH of SEQ ID NO: 18, and variable light chain domain VL of SEQ ID NO: 22 (corresponding to the VH and VL domains of <PD-L1> “243.55.H12” as disclosed herein);
variable heavy chain domain VH of SEQ ID NO: 18, and variable light chain domain VL of SEQ ID NO: 23 (corresponding to the VH and VL domains of <PD-L1> “243.55.H37” as disclosed herein);
variable heavy chain domain VH of SEQ ID NO: 18, and variable light chain domain VL of SEQ ID NO: 24 (corresponding to the VH and VL domains of <PD-L1> “243.55.H70” as disclosed herein);
variable heavy chain domain VH of SEQ ID NO: 18, and variable light chain domain VL of SEQ ID NO: 25 (corresponding to the VH and VL domains of <PD-L1> “243.55.H89” as disclosed herein);
variable heavy chain domain VH of SEQ ID NO: 18, and variable light chain domain VL of SEQ ID NO: 26 (corresponding to the VH and VL domains of <PD-L1> “243.55.S1” as disclosed herein);
variable heavy chain domain VH of SEQ ID NO: 18, and variable light chain domain VL of SEQ ID NO: 27 (corresponding to the VH and VL domains of <PD-L1> “243.55.5” as disclosed herein);
variable heavy chain domain VH of SEQ ID NO: 18, and variable light chain domain VL of SEQ ID NO: 28 (corresponding to the VH and VL domains of <PD-L1> “243.55.8” as disclosed herein);
variable heavy chain domain VH of SEQ ID NO: 18, and variable light chain domain VL of SEQ ID NO: 29 (corresponding to the VH and VL domains of <PD-L1> “243.55.30” as disclosed herein);
variable heavy chain domain VH of SEQ ID NO: 18, and variable light chain domain VL of SEQ ID NO: 30 (corresponding to the VH and VL domains of <PD-L1> “243.55.34” as disclosed herein);
variable heavy chain domain VH of SEQ ID NO: 18, and variable light chain domain VL of SEQ ID NO: 31 (corresponding to the VH and VL domains of <PD-L1> “243.55.S37” as disclosed herein);
variable heavy chain domain VH of SEQ ID NO: 18, and variable light chain domain VL of SEQ ID NO: 32 (corresponding to the VH and VL domains of <PD-L1> “243.55.49” as disclosed herein);
variable heavy chain domain VH of SEQ ID NO: 18, and variable light chain domain VL of SEQ ID NO: 33 (corresponding to the VH and VL domains of <PD-L1> “243.55.51” as disclosed herein);
variable heavy chain domain VH of SEQ ID NO: 18, and variable light chain domain VL of SEQ ID NO: 34 (corresponding to the VH and VL domains of <PD-L1> “243.55.62” as disclosed herein); and
variable heavy chain domain VH of SEQ ID NO: 19, and variable light chain domain VL of SEQ ID NO: 35 (corresponding to the VH and VL domains of <PD-L1> “243.55.84” as disclosed herein).

In one embodiment of the invention, the combination therapy of the present invention uses the antibody that binds to ANG-2 comprising the variable heavy chain domain VH of SEQ ID NO:4, and the variable light chain domain VL of SEQ ID NO: 5 (corresponding to the VH and VL domains of <ANG-2> LC06 as disclosed herein); and the antibody that binds to PD-L1 comprising the variable heavy chain domain VH of SEQ ID NO: 17, and the variable light chain domain VL of SEQ ID NO: 20 (corresponding to the VH and VL domains of <PD-L1> “243.55.S70” as disclosed herein).

In one embodiment of the invention, the combination therapy of the present invention uses the antibody that binds to ANG-2 comprising the variable heavy chain domain VH of SEQ ID NO: 10, and the variable light chain domain VL of SEQ ID NO: 5 (corresponding to the VH and VL domains of <ANG-2> E6Q as disclosed herein); and an antibody that binds to PD-L1 comprising variable domain amino acid sequences, selected from the group of:
variable heavy chain domain VH of SEQ ID NO: 17, and variable light chain domain VL of SEQ ID NO: 20 (corresponding to the VH and VL domains of <PD-L1> “243.55.S70” as disclosed herein);
variable heavy chain domain VH of SEQ ID NO: 18, and variable light chain domain VL of SEQ ID NO: 21 (corresponding to the VH and VL domains of <PD-L1> “243.55.H1” as disclosed herein);
variable heavy chain domain VH of SEQ ID NO: 18, and variable light chain domain VL of SEQ ID NO: 22 (corresponding to the VH and VL domains of <PD-L1> “243.55.H12” as disclosed herein);
variable heavy chain domain VH of SEQ ID NO: 18, and variable light chain domain VL of SEQ ID NO: 23 (corresponding to the VH and VL domains of <PD-L1> “243.55.H37” as disclosed herein);
variable heavy chain domain VH of SEQ ID NO: 18, and variable light chain domain VL of SEQ ID NO: 24 (corresponding to the VH and VL domains of <PD-L1> “243.55.H70” as disclosed herein);
variable heavy chain domain VH of SEQ ID NO: 18, and variable light chain domain VL of SEQ ID NO: 25 (corresponding to the VH and VL domains of <PD-L1> “243.55.H89” as disclosed herein);
variable heavy chain domain VH of SEQ ID NO: 18, and variable light chain domain VL of SEQ ID NO: 26 (corresponding to the VH and VL domains of <PD-L1> “243.55.S1” as disclosed herein);
variable heavy chain domain VH of SEQ ID NO: 18, and variable light chain domain VL of SEQ ID NO: 27 (corresponding to the VH and VL domains of <PD-L1> “243.55.5” as disclosed herein);
variable heavy chain domain VH of SEQ ID NO: 18, and variable light chain domain VL of SEQ ID NO: 28 (corresponding to the VH and VL domains of <PD-L1> “243.55.8” as disclosed herein);
variable heavy chain domain VH of SEQ ID NO: 18, and variable light chain domain VL of SEQ ID NO: 29 (corresponding to the VH and VL domains of <PD-L1> “243.55.30” as disclosed herein);
variable heavy chain domain VH of SEQ ID NO: 18, and variable light chain domain VL of SEQ ID NO: 30 (corresponding to the VH and VL domains of <PD-L1> “243.55.34” as disclosed herein);
variable heavy chain domain VH of SEQ ID NO: 18, and variable light chain domain VL of SEQ ID NO: 31 (corresponding to the VH and VL domains of <PD-L1> “243.55.S37” as disclosed herein);
variable heavy chain domain VH of SEQ ID NO: 18, and variable light chain domain VL of SEQ ID NO: 32 (corresponding to the VH and VL domains of <PD-L1> “243.55.49” as disclosed herein);
variable heavy chain domain VH of SEQ ID NO: 18, and variable light chain domain VL of SEQ ID NO: 33 (corresponding to the VH and VL domains of <PD-L1> “243.55.51” as disclosed herein);
variable heavy chain domain VH of SEQ ID NO: 18, and variable light chain domain VL of SEQ ID NO: 34 (corresponding to the VH and VL domains of <PD-L1> “243.55.62” as disclosed herein); and
variable heavy chain domain VH of SEQ ID NO: 19, and variable light chain domain VL of SEQ ID NO: 35 (corresponding to the VH and VL domains of <PD-L1> “243.55.84” as disclosed herein).

In one embodiment of the invention, the combination therapy of the present invention uses the antibody that binds to ANG-2 comprising the variable heavy chain domain VH of SEQ ID NO: 10, and the variable light chain domain VL of SEQ ID NO: 5 (corresponding to the VH and VL domains of <ANG-2> E6Q as disclosed herein); and the antibody that binds to PD-L1 comprising the variable heavy chain domain VH of SEQ ID NO: 17, and the variable light chain domain VL of SEQ ID NO: 20 (corresponding to the VH and VL domains of <PD-L1> “243.55.S70” as disclosed herein).

In one embodiment of the invention, the combination therapy of the present invention uses an antibody that binds to ANG-2 and VEGF, the antibody comprising at least one binding site that binds to ANG-2 comprising the variable heavy chain domain VH of SEQ ID NO: 10, and the variable light chain domain VL of SEQ ID NO: 5 (corresponding to the VH and VL domains of <ANG-2> E6Q as disclosed herein); and at least one binding site that binds to VEGF comprising the variable heavy chain domain VH of SEQ ID NO: 8, and the variable light chain domain VL of SEQ ID NO: 9 (corresponding to the VH and VL domains of <VEGF> bevacizumab as disclosed herein). In one embodiment, said antibody that binds to ANG-2 and VEGF is a bispecific antibody.

In one embodiment of the invention, the combination therapy of the present invention uses an antibody that binds to ANG-2 and VEGF, the antibody comprising at least one binding site that binds to ANG-2 comprising the variable heavy chain domain VH of SEQ ID NO: 10, and the variable light chain domain VL of SEQ ID NO: 5 (corresponding to the VH and VL domains of <ANG-2> E6Q as disclosed herein); and at least one binding site that binds to VEGF comprising the variable heavy chain domain VH of SEQ ID NO: 6, and the variable light chain domain VL of SEQ ID NO: 7 (corresponding to the VH and VL domains of <VEGF> B20.4.1 as disclosed herein). In one embodiment, said antibody that binds to ANG-2 and VEGF is a bispecific antibody.

In one embodiment of the invention, the combination therapy of the present invention uses a bispecific antibody that binds to ANG-2 and VEGF, the antibody comprising: a heavy chain comprising SEQ ID NO: 11 (corresponding to the ANG-2 heavy chain of <ANG-2/VEGF> E6Q/bevacizumab as disclosed herein), a light chain comprising SEQ ID NO: 12 (corresponding to the ANG-2 light chain of <ANG-2/VEGF> E6Q/bevacizumab as disclosed herein), a heavy chain comprising SEQ ID NO: 15 (corresponding to the VEGF heavy chain of E6Q/bevacizumab as disclosed herein), and a light chain comprising SEQ ID NO: 16 (corresponding to the VEGF light chain of <ANG-2/VEGF> E6Q/bevacizumab as disclosed herein).

In one embodiment of the invention, the combination therapy of the present invention uses a bispecific antibody that binds to ANG-2 and VEGF, the antibody comprising: a heavy chain comprising SEQ ID NO: 11 (corresponding to the ANG-2 heavy chain of <ANG-2/VEGF> E6Q/B20.4.1 as disclosed herein), a light chain comprising SEQ ID NO: 12 (corresponding to the ANG-2 light chain of <ANG-2/VEGF> E6Q/B20.4.1 as disclosed herein), a heavy chain comprising SEQ ID NO: 13 (corresponding to the VEGF heavy chain of <ANG-2/VEGF> E6Q/B20.4.1 as disclosed herein), and a light chain comprising SEQ ID NO: 14 (corresponding to the VEGF light chain of <ANG-2/VEGF> E6Q/B20.4.1 as disclosed herein).

In one embodiment of the invention, the combination therapy of the present invention uses an antibody that binds to ANG-2 and VEGF, the antibody comprising at least one binding site that binds to ANG-2 comprising the variable heavy chain domain VH of SEQ ID NO: 10, and the variable light chain domain VL of SEQ ID NO: 5 (corresponding to the VH and VL domains of <ANG-2> E6Q as disclosed herein); and at least one binding site that binds to VEGF comprising the variable heavy chain domain VH of SEQ ID NO: 8, and the variable light chain domain VL of SEQ ID NO: 9 (corresponding to the VH and VL domains of <VEGF> bevacizumab as disclosed herein); and an antibody that binds to PD-L1 comprising variable domain amino acid sequences, selected from the group of:
variable heavy chain domain VH of SEQ ID NO: 17, and variable light chain domain VL of SEQ ID NO: 20 (corresponding to the VH and VL domains of <PD-L1> “243.55.S70” as disclosed herein);
variable heavy chain domain VH of SEQ ID NO: 18, and variable light chain domain VL of SEQ ID NO: 21 (corresponding to the VH and VL domains of <PD-L1> “243.55.H1” as disclosed herein);
variable heavy chain domain VH of SEQ ID NO: 18, and variable light chain domain VL of SEQ ID NO: 22 (corresponding to the VH and VL domains of <PD-L1> “243.55.H12” as disclosed herein);
variable heavy chain domain VH of SEQ ID NO: 18, and variable light chain domain VL of SEQ ID NO: 23 (corresponding to the VH and VL domains of <PD-L1> “243.55.H37” as disclosed herein);
variable heavy chain domain VH of SEQ ID NO: 18, and variable light chain domain VL of SEQ ID NO: 24 (corresponding to the VH and VL domains of <PD-L1> “243.55.H70” as disclosed herein);
variable heavy chain domain VH of SEQ ID NO: 18, and variable light chain domain VL of SEQ ID NO: 25 (corresponding to the VH and VL domains of <PD-L1> “243.55.H89” as disclosed herein);
variable heavy chain domain VH of SEQ ID NO: 18, and variable light chain domain VL of SEQ ID NO: 26 (corresponding to the VH and VL domains of <PD-L1> “243.55.S1” as disclosed herein);
variable heavy chain domain VH of SEQ ID NO: 18, and variable light chain domain VL of SEQ ID NO: 27 (corresponding to the VH and VL domains of <PD-L1> “243.55.5” as disclosed herein);
variable heavy chain domain VH of SEQ ID NO: 18, and variable light chain domain VL of SEQ ID NO: 28 (corresponding to the VH and VL domains of <PD-L1> “243.55.8” as disclosed herein);
variable heavy chain domain VH of SEQ ID NO: 18, and variable light chain domain VL of SEQ ID NO: 29 (corresponding to the VH and VL domains of <PD-L1> “243.55.30” as disclosed herein);
variable heavy chain domain VH of SEQ ID NO: 18, and variable light chain domain VL of SEQ ID NO: 30 (corresponding to the VH and VL domains of <PD-L1> “243.55.34” as disclosed herein);
variable heavy chain domain VH of SEQ ID NO: 18, and variable light chain domain VL of SEQ ID NO: 31 (corresponding to the VH and VL domains of <PD-L1> “243.55.S37” as disclosed herein);
variable heavy chain domain VH of SEQ ID NO: 18, and variable light chain domain VL of SEQ ID NO: 32 (corresponding to the VH and VL domains of <PD-L1> “243.55.49” as disclosed herein);
variable heavy chain domain VH of SEQ ID NO: 18, and variable light chain domain VL of SEQ ID NO: 33 (corresponding to the VH and VL domains of <PD-L1> “243.55.51” as disclosed herein);
variable heavy chain domain VH of SEQ ID NO: 18, and variable light chain domain VL of SEQ ID NO: 34 (corresponding to the VH and VL domains of <PD-L1> “243.55.62” as disclosed herein); and
variable heavy chain domain VH of SEQ ID NO: 19, and variable light chain domain VL of SEQ ID NO: 35 (corresponding to the VH and VL domains of <PD-L1> “243.55.84” as disclosed herein).

In one embodiment of the invention, the combination therapy of the present invention uses an antibody that binds to ANG-2 and VEGF, the antibody comprising at least one binding site that binds to ANG-2 comprising the variable heavy chain domain VH of SEQ ID NO: 10, and the variable light chain domain VL of SEQ ID NO: 5 (corresponding to the VH and VL domains of <ANG-2> E6Q as disclosed herein); and at least one binding site that binds to VEGF comprising the variable heavy chain domain VH of SEQ ID NO: 8, and the variable light chain domain VL of SEQ ID NO: 9 (corresponding to the VH and VL domains of <VEGF> bevacizumab as disclosed herein); and the antibody that binds to PD-L1 comprising the variable heavy chain domain VH of SEQ ID NO: 17, and the variable light chain domain VL of SEQ ID NO: 20 (corresponding to the VH and VL domains of <PD-L1> “243.55.S70” as disclosed herein). In one embodiment, said antibody that binds to ANG-2 and VEGF is a bispecific antibody.

In one embodiment of the invention, the combination therapy of the present invention uses an antibody that binds to ANG-2 and VEGF, the antibody comprising at least one binding site that binds to ANG-2 comprising the variable heavy chain domain VH of SEQ ID NO: 10, and the variable light chain domain VL of SEQ ID NO: 5 (corresponding to the VH and VL domains of <ANG-2> E6Q as disclosed herein); and at least one binding site that binds to VEGF comprising the variable heavy chain domain VH of SEQ ID NO: 6, and the variable light chain domain VL of SEQ ID NO: 7 (corresponding to the VH and VL domains of <VEGF> B20.4.1 as disclosed herein); and an antibody that binds to PD-L1 comprising variable domain amino acid sequences, selected from the group of:
variable heavy chain domain VH of SEQ ID NO: 17, and variable light chain domain VL of SEQ ID NO: 20 (corresponding to the VH and VL domains of <PD-L1> “243.55.S70” as disclosed herein);
variable heavy chain domain VH of SEQ ID NO: 18, and variable light chain domain VL of SEQ ID NO: 21 (corresponding to the VH and VL domains of <PD-L1> “243.55.H1” as disclosed herein);
variable heavy chain domain VH of SEQ ID NO: 18, and variable light chain domain VL of SEQ ID NO: 22 (corresponding to the VH and VL domains of <PD-L1> “243.55.H12” as disclosed herein);
variable heavy chain domain VH of SEQ ID NO: 18, and variable light chain domain VL of SEQ ID NO: 23 (corresponding to the VH and VL domains of <PD-L1> “243.55.H37” as disclosed herein);
variable heavy chain domain VH of SEQ ID NO: 18, and variable light chain domain VL of SEQ ID NO: 24 (corresponding to the VH and VL domains of <PD-L1> “243.55.H70” as disclosed herein);
variable heavy chain domain VH of SEQ ID NO: 18, and variable light chain domain VL of SEQ ID NO: 25 (corresponding to the VH and VL domains of <PD-L1> “243.55.H89” as disclosed herein);
variable heavy chain domain VH of SEQ ID NO: 18, and variable light chain domain VL of SEQ ID NO: 26 (corresponding to the VH and VL domains of <PD-L1> “243.55.S1” as disclosed herein);
variable heavy chain domain VH of SEQ ID NO: 18, and variable light chain domain VL of SEQ ID NO: 27 (corresponding to the VH and VL domains of <PD-L1> “243.55.5” as disclosed herein);
variable heavy chain domain VH of SEQ ID NO: 18, and variable light chain domain VL of SEQ ID NO: 28 (corresponding to the VH and VL domains of <PD-L1> “243.55.8” as disclosed herein);
variable heavy chain domain VH of SEQ ID NO: 18, and variable light chain domain VL of SEQ ID NO: 29 (corresponding to the VH and VL domains of <PD-L1> “243.55.30” as disclosed herein);
variable heavy chain domain VH of SEQ ID NO: 18, and variable light chain domain VL of SEQ ID NO: 30 (corresponding to the VH and VL domains of <PD-L1> “243.55.34” as disclosed herein);
variable heavy chain domain VH of SEQ ID NO: 18, and variable light chain domain VL of SEQ ID NO: 31 (corresponding to the VH and VL domains of <PD-L1> “243.55.S37” as disclosed herein);
variable heavy chain domain VH of SEQ ID NO: 18, and variable light chain domain VL of SEQ ID NO: 32 (corresponding to the VH and VL domains of <PD-L1> “243.55.49” as disclosed herein);
variable heavy chain domain VH of SEQ ID NO: 18, and variable light chain domain VL of SEQ ID NO: 33 (corresponding to the VH and VL domains of <PD-L1> “243.55.51” as disclosed herein);
variable heavy chain domain VH of SEQ ID NO: 18, and variable light chain domain VL of SEQ ID NO: 34 (corresponding to the VH and VL domains of <PD-L1> “243.55.62” as disclosed herein); and
variable heavy chain domain VH of SEQ ID NO: 19, and variable light chain domain VL of SEQ ID NO: 35 (corresponding to the VH and VL domains of <PD-L1> “243.55.84” as disclosed herein).

In one embodiment of the invention, the combination therapy of the present invention uses an antibody that binds to ANG-2 and VEGF, the antibody comprising at least one binding site that binds to ANG-2 comprising the variable heavy chain domain VH of SEQ ID NO: 10, and the variable light chain domain VL of SEQ ID NO: 5 (corresponding to the VH and VL domains of <ANG-2> E6Q as disclosed herein); and at least one binding site that binds to VEGF comprising the variable heavy chain domain VH of SEQ ID NO: 6, and the variable light chain domain VL of SEQ ID NO: 7 (corresponding to the VH and VL domains of <VEGF> B20.4.1 as disclosed herein); and the antibody that binds to PD-L1 comprising the variable heavy chain domain VH of SEQ ID NO: 17, and the variable light chain domain VL of SEQ ID NO: 20 (corresponding to the VH and VL domains of <PD-L1> “243.55.S70” as disclosed herein). In one embodiment, said antibody that binds to ANG-2 and VEGF is a bispecific antibody.

In one embodiment of the invention, the combination therapy of the present invention uses a bispecific antibody that binds to ANG-2 and VEGF, the antibody comprising: a heavy chain comprising SEQ ID NO: 11 (corresponding to the ANG-2 heavy chain of <ANG-2/VEGF> E6Q/bevacizumab as disclosed herein), a light chain comprising SEQ ID NO: 12 (corresponding to the ANG-2 light chain of <ANG-2/VEGF> E6Q/bevacizumab as disclosed herein), a heavy chain comprising SEQ ID NO: 15 (corresponding to the VEGF heavy chain of E6Q/bevacizumab as disclosed herein), and a light chain comprising SEQ ID NO: 16 (corresponding to the VEGF light chain of <ANG-2/VEGF> E6Q/bevacizumab as disclosed herein); and an antibody that binds to PD-L1 comprising variable domain amino acid sequences, selected from the group of:
variable heavy chain domain VH of SEQ ID NO: 17, and variable light chain domain VL of SEQ ID NO: 20 (corresponding to the VH and VL domains of <PD-L1> “243.55.S70” as disclosed herein);
variable heavy chain domain VH of SEQ ID NO: 18, and variable light chain domain VL of SEQ ID NO: 21 (corresponding to the VH and VL domains of <PD-L1> “243.55.H1” as disclosed herein);
variable heavy chain domain VH of SEQ ID NO: 18, and variable light chain domain VL of SEQ ID NO: 22 (corresponding to the VH and VL domains of <PD-L1> “243.55.H12” as disclosed herein);
variable heavy chain domain VH of SEQ ID NO: 18, and variable light chain domain VL of SEQ ID NO: 23 (corresponding to the VH and VL domains of <PD-L1> “243.55.H37” as disclosed herein);
variable heavy chain domain VH of SEQ ID NO: 18, and variable light chain domain VL of SEQ ID NO: 24 (corresponding to the VH and VL domains of <PD-L1> “243.55.H70” as disclosed herein);
variable heavy chain domain VH of SEQ ID NO: 18, and variable light chain domain VL of SEQ ID NO: 25 (corresponding to the VH and VL domains of <PD-L1> “243.55.H89” as disclosed herein);
variable heavy chain domain VH of SEQ ID NO: 18, and variable light chain domain VL of SEQ ID NO: 26 (corresponding to the VH and VL domains of <PD-L1> “243.55.S1” as disclosed herein);
variable heavy chain domain VH of SEQ ID NO: 18, and variable light chain domain VL of SEQ ID NO: 27 (corresponding to the VH and VL domains of <PD-L1> “243.55.5” as disclosed herein);
variable heavy chain domain VH of SEQ ID NO: 18, and variable light chain domain VL of SEQ ID NO: 28 (corresponding to the VH and VL domains of <PD-L1> “243.55.8” as disclosed herein);
variable heavy chain domain VH of SEQ ID NO: 18, and variable light chain domain VL of SEQ ID NO: 29 (corresponding to the VH and VL domains of <PD-L1> “243.55.30” as disclosed herein);
variable heavy chain domain VH of SEQ ID NO: 18, and variable light chain domain VL of SEQ ID NO: 30 (corresponding to the VH and VL domains of <PD-L1> “243.55.34” as disclosed herein);
variable heavy chain domain VH of SEQ ID NO: 18, and variable light chain domain VL of SEQ ID NO: 31 (corresponding to the VH and VL domains of <PD-L1> “243.55.S37” as disclosed herein);
variable heavy chain domain VH of SEQ ID NO: 18, and variable light chain domain VL of SEQ ID NO: 32 (corresponding to the VH and VL domains of <PD-L1> “243.55.49” as disclosed herein);
variable heavy chain domain VH of SEQ ID NO: 18, and variable light chain domain VL of SEQ ID NO: 33 (corresponding to the VH and VL domains of <PD-L1> “243.55.51” as disclosed herein);
variable heavy chain domain VH of SEQ ID NO: 18, and variable light chain domain VL of SEQ ID NO: 34 (corresponding to the VH and VL domains of <PD-L1> “243.55.62” as disclosed herein); and
variable heavy chain domain VH of SEQ ID NO: 19, and variable light chain domain VL of SEQ ID NO: 35 (corresponding to the VH and VL domains of <PD-L1> “243.55.84” as disclosed herein).

In one embodiment of the invention, the combination therapy of the present invention uses a bispecific antibody that binds to ANG-2 and VEGF, the antibody comprising: a heavy chain comprising SEQ ID NO: 11 (corresponding to the ANG-2 heavy chain of <ANG-2/VEGF> E6Q/bevacizumab as disclosed herein), a light chain comprising SEQ ID NO: 12 (corresponding to the ANG-2 light chain of <ANG-2/VEGF> E6Q/bevacizumab as disclosed herein), a heavy chain comprising SEQ ID NO: 15 (corresponding to the VEGF heavy chain of E6Q/bevacizumab as disclosed herein), and a light chain comprising SEQ ID NO: 16 (corresponding to the VEGF light chain of <ANG-2/VEGF> E6Q/bevacizumab as disclosed herein); and the antibody that binds to PD-L1 comprising the variable heavy chain domain VH of SEQ ID NO: 17, and the variable light chain domain VL of SEQ ID NO: 20 (corresponding to the VH and VL domains of <PD-L1> “243.55.S70” as disclosed herein).

In one embodiment of the invention, the combination therapy of the present invention uses a bispecific antibody that binds to ANG-2 and VEGF, the antibody comprising: a heavy chain comprising SEQ ID NO: 11 (corresponding to the ANG-2 heavy chain of <ANG-2/VEGF> E6Q/B20.4.1 as disclosed herein), a light chain comprising SEQ ID NO: 12 (corresponding to the ANG-2 light chain of <ANG-2/VEGF> E6Q/B20.4.1 as disclosed herein), a heavy chain comprising SEQ ID NO: 13 (corresponding to the VEGF heavy chain of <ANG-2/VEGF> E6Q/B20.4.1 as disclosed herein), and a light chain comprising SEQ ID NO: 14 (corresponding to the VEGF light chain of <ANG-2/VEGF> E6Q/B20.4.1 as disclosed herein); and an antibody that binds to PD-L1 comprising variable domain amino acid sequences, selected from the group of:
variable heavy chain domain VH of SEQ ID NO: 17, and variable light chain domain VL of SEQ ID NO: 20 (corresponding to the VH and VL domains of <PD-L1> “243.55.S70” as disclosed herein);
variable heavy chain domain VH of SEQ ID NO: 18, and variable light chain domain VL of SEQ ID NO: 21 (corresponding to the VH and VL domains of <PD-L1> “243.55.H1” as disclosed herein);
variable heavy chain domain VH of SEQ ID NO: 18, and variable light chain domain VL of SEQ ID NO: 22 (corresponding to the VH and VL domains of <PD-L1> “243.55.H12” as disclosed herein);
variable heavy chain domain VH of SEQ ID NO: 18, and variable light chain domain VL of SEQ ID NO: 23 (corresponding to the VH and VL domains of <PD-L1> “243.55.H37” as disclosed herein);
variable heavy chain domain VH of SEQ ID NO: 18, and variable light chain domain VL of SEQ ID NO: 24 (corresponding to the VH and VL domains of <PD-L1> “243.55.H70” as disclosed herein);
variable heavy chain domain VH of SEQ ID NO: 18, and variable light chain domain VL of SEQ ID NO: 25 (corresponding to the VH and VL domains of <PD-L1> “243.55.H89” as disclosed herein);
variable heavy chain domain VH of SEQ ID NO: 18, and variable light chain domain VL of SEQ ID NO: 26 (corresponding to the VH and VL domains of <PD-L1> “243.55.S1” as disclosed herein);
variable heavy chain domain VH of SEQ ID NO: 18, and variable light chain domain VL of SEQ ID NO: 27 (corresponding to the VH and VL domains of <PD-L1> “243.55.5” as disclosed herein);
variable heavy chain domain VH of SEQ ID NO: 18, and variable light chain domain VL of SEQ ID NO: 28 (corresponding to the VH and VL domains of <PD-L1> “243.55.8” as disclosed herein);
variable heavy chain domain VH of SEQ ID NO: 18, and variable light chain domain VL of SEQ ID NO: 29 (corresponding to the VH and VL domains of <PD-L1> “243.55.30” as disclosed herein);
variable heavy chain domain VH of SEQ ID NO: 18, and variable light chain domain VL of SEQ ID NO: 30 (corresponding to the VH and VL domains of <PD-L1> “243.55.34” as disclosed herein);
variable heavy chain domain VH of SEQ ID NO: 18, and variable light chain domain VL of SEQ ID NO: 31 (corresponding to the VH and VL domains of <PD-L1> “243.55.S37” as disclosed herein);
variable heavy chain domain VH of SEQ ID NO: 18, and variable light chain domain VL of SEQ ID NO: 32 (corresponding to the VH and VL domains of <PD-L1> “243.55.49” as disclosed herein);
variable heavy chain domain VH of SEQ ID NO: 18, and variable light chain domain VL of SEQ ID NO: 33 (corresponding to the VH and VL domains of <PD-L1> “243.55.51” as disclosed herein);
variable heavy chain domain VH of SEQ ID NO: 18, and variable light chain domain VL of SEQ ID NO: 34 (corresponding to the VH and VL domains of <PD-L1> “243.55.62” as disclosed herein); and
variable heavy chain domain VH of SEQ ID NO: 19, and variable light chain domain VL of SEQ ID NO: 35 (corresponding to the VH and VL domains of <PD-L1> “243.55.84” as disclosed herein).

In one embodiment of the invention, the combination therapy of the present invention uses a bispecific antibody that binds to ANG-2 and VEGF, the antibody comprising: a heavy chain comprising SEQ ID NO: 11 (corresponding to the ANG-2 heavy chain of <ANG-2/VEGF> E6Q/B20.4.1 as disclosed herein), a light chain comprising SEQ ID NO: 12 (corresponding to the ANG-2 light chain of <ANG-2/VEGF> E6Q/B20.4.1 as disclosed herein), a heavy chain comprising SEQ ID NO: 13 (corresponding to the VEGF heavy chain of <ANG-2/VEGF> E6Q/B20.4.1 as disclosed herein), and a light chain comprising SEQ ID NO: 14 (corresponding to the VEGF light chain of <ANG-2/VEGF> E6Q/B20.4.1 as disclosed herein); and the antibody that binds to PD-L1 comprising the variable heavy chain domain VH of SEQ ID NO: 17, and the variable light chain domain VL of SEQ ID NO: 20 (corresponding to the VH and VL domains of <PD-L1> “243.55.S70” as disclosed herein).

In one embodiment of the invention, the antibody that binds to ANG-2 that is used in a combination therapy according to the invention is a human antibody or a humanized antibody.

In one embodiment of the invention, the antibody that binds to ANG-2 that is used in a combination therapy according to the invention is of IgG isotype. In one embodiment of the invention, the antibody that binds to ANG-2 that is used in a combination therapy according to the invention is of IgG1, IgG2, IgG3 or IgG4 isotype. In one preferred embodiment of the invention, the antibody that binds to ANG-2 that is used in a combination therapy according to the invention is of human IgG1 or of human IgG4 isotype. In one preferred embodiment of the invention, the antibody that binds to ANG-2 that is used in a combination therapy according to the invention is of human IgG1 isotype. In one preferred embodiment of the invention, the antibody that binds to ANG-2 that is used in a combination therapy according to the invention is of human IgG1 isotype with a mutation at positions L234A and L235A. In another preferred embodiment of the invention, the antibody that binds to ANG-2 that is used in a combination therapy according to the invention is of human IgG4 isotype. In one preferred embodiment of the invention, the antibody that binds to ANG-2 that is used in a combination therapy according to the invention is of human IgG4 isotype with a mutation at position S228P.

In one embodiment of the invention, the antibody that binds to ANG-2 that is used in a combination therapy according to the invention

In one embodiment of the invention, the antibody that binds to ANG-2 that is used in a combination therapy according to the invention specifically binds to human ANG-2 with a K_{D} value of less than 1.0×10⁻⁸ mol/l, as determined by surface plasmon resonance.

In one embodiment of the invention, the antibody that binds to ANG-2 that is used in a combination therapy according to the invention inhibits the interaction of human ANG-2 with TIE2 receptor with an IC50 of 15 nM or less.

In one embodiment of the invention, the antibody that binds to PD-L1 that is used in a combination therapy according to the invention is a human antibody or a humanized antibody.

In one embodiment of the invention, the antibody that binds to PD-L1 that is used in a combination therapy according to the invention is of IgG isotype. In one embodiment of the invention, the antibody that binds to PD-L1 that is used in a combination therapy according to the invention is of IgG1, IgG2, IgG3 or IgG4 isotype. In one preferred embodiment of the invention, the antibody that binds to PD-L1 that is used in a combination therapy according to the invention is of human IgG1 or of human IgG4 isotype. In one preferred embodiment of the invention, the antibody that binds to PD-L1 that is used in a combination therapy according to the invention is of human IgG1 isotype. In one preferred embodiment of the invention, the antibody that binds to PD-L1 that is used in a combination therapy according to the invention is of human IgG1 isotype with a mutation at positions L234A and L235A. In another preferred embodiment of the invention, the antibody that binds to PD-L1 that is used in a combination therapy according to the invention is of human IgG4 isotype. In one preferred embodiment of the invention, the antibody that binds to PD-L1 that is used in a combination therapy according to the invention is of human IgG4 isotype with a mutation at position S228P.

In one embodiment of the invention, the antibody that binds to PD-L1 that is used in a combination therapy according to the invention specifically binds to human PD-L1 with a K_{D} value of less than 1.0×10⁻⁸ mol/l, as determined by surface plasmon resonance.

In one embodiment of the invention, the antibody that binds to VEGF that is used in a combination therapy according to one embodiment of the invention is a human antibody or a humanized antibody.

In one embodiment of the invention, the antibody that binds to VEGF that is used in a combination therapy according to one embodiment of the invention is of IgG isotype. In one embodiment of the invention, the antibody that binds to VEGF that is used in a combination therapy according to the invention is of IgG1, IgG2, IgG3 or IgG4 isotype. In one preferred embodiment of the invention, the antibody that binds to VEGF that is used in a combination therapy according to the invention is of human IgG1 or of human IgG4 isotype. In one preferred embodiment of the invention, the antibody that binds to VEGF that is used in a combination therapy according to the invention is of human IgG1 isotype. In one preferred embodiment of the invention, the antibody that binds to VEGF that is used in a combination therapy according to the invention is of human IgG1 isotype with a mutation at positions L234A and L235A. In another preferred embodiment of the invention, the antibody that binds to VEGF that is used in a combination therapy according to the invention is of human IgG4 isotype. In one preferred embodiment of the invention, the antibody that binds to VEGF that is used in a combination therapy according to the invention is of human IgG4 isotype with a mutation at position S228P.

In one embodiment of the invention, the antibody that binds to VEGF that is used in a combination therapy according to the invention specifically binds to human VEGF with a K_{D} value of less than 1.0×10⁻⁸ mol/l, as determined by surface plasmon resonance.

In one embodiment of the invention, the antibodies that bind to ANG-2 and to PD-L1 that are used in a combination therapy according to the invention are both human antibodies or a humanized antibodies.

In one embodiment of the invention, the antibodies that bind to ANG-2 and to PD-L1 that are used in a combination therapy according to the invention are both of IgG isotype.

In one embodiment of the invention, the antibodies that bind to human ANG-2 and to human PD-L1 that are used in a combination therapy according to the invention both bind to their respective targets, i.e. human ANG-2 and human PD-L1, with a K_{D} value of less than 1.0×10⁻⁸ mol/l, as determined by surface plasmon resonance.

In one embodiment of the invention, the antibodies that bind to ANG-2, to PD-L1 and to VEGF that are used in a combination therapy according to the invention are human antibodies or humanized antibodies, respectively.

In one embodiment of the invention, the antibodies that bind to ANG-2, to PD-L1 and to VEGF that are used in a combination therapy according to the invention are of IgG isotype.

In one embodiment of the invention, the antibodies that bind to human ANG-2, to human PD-L1 and to human VEGF that are used in a combination therapy according to the invention bind to their respective targets, i.e. human ANG-2, human PD-L1 and human VEGF, with a K_{D} value of less than 1.0×10⁻⁸ mol/l, as determined by surface plasmon resonance.

### Therapeutic Indications

In one embodiment of the invention, wherein the combination therapy is used for treating cancer, delaying progression of cancer, or prolonging the survival of a patient suffering from cancer, the cancer is selected from the group of lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, and lymphocytic leukemia.

In one embodiment of the invention, wherein the combination therapy is used for treating cancer, delaying progression of cancer, or prolonging the survival of a patient suffering from cancer, the cancer is a solid tumor.

In one embodiment of the invention, the combination therapy is used for

a) treating a solid tumor,

b) delaying progression of a solid tumor, or

c) prolonging the survival of a patient suffering from a solid tumor.

Administration in further Combination Therapies

In one embodiment of a combination therapy according to the invention, the combination therapy of the antibody that binds to ANG-2 and the antibody that binds to PD-L1 is co-administered with a chemotherapeutic agent.

In one embodiment of a combination therapy according to the invention, the combination therapy of the antibody that binds to ANG-2, the antibody that binds to VEGF and the antibody that binds to PD-L1 is co-administered with a chemotherapeutic agent.

In one embodiment, said chemotherapeutic agent is selected from the group of: anti-neoplastic agents including nitrogen mustards (in one embodiment mechlorethamine, cyclophosphamide, ifosfamide, melphalan, or chlorambucil); nitrosoureas (in one embodiment carmustine (BCNU), lomustine (CCNU), or semustine (methyl-CCNU)); Temodal™ (temozolamide), ethylenimines/methylmel amine (in one embodiment thriethylenemel amine (TEM), triethylene, thiophosphoramide (thiotepa), or hexamethylmelamine (HMM, altretamine)); alkyl sulfonates (in one embodiment busulfan); triazines (in one embodiment dacarbazine (DTIC)); antimetabolites including folic acid analogs (in one embodiment methotrexate or trimetrexate), pyrimidine analogs (in one embodiment 5-fluorouracil (5FU), fluorodeoxyuridine, gemcitabine, cytosine arabinoside (AraC, cytarabine), 5-azacytidine, 2,2′-difluorodeoxycytidine), purine analogs (in one embodiment 6-mercarhotopurine, 6-thioguamne, azathioprine, T-deoxycoformycin (pentostatin), erythrohydroxynonyladenine (EHNA), fludarabine phosphate, or 2-chlorodeoxyadenosine (cladribine, 2-CdA)); natural products including antimitotic drugs (in one embodiment paclitaxel, vinca alkaloids including vinblastine (VLB), vincristine, vinorelbine, taxotere, estramustine, or estramustine phosphate); pipodophylotoxins (in one embodiment etoposide or teniposide); antibiotics (in one embodiment actinomycin D, daunomycin (rubidomycin), doxorubicin, mitoxantrone, idarubicin, bleomycins, plicamycin (mithramycin), mitomycin C, and actinomycin); enzymes (in one embodiment L-asparaginase); biological response modifiers (in one embodiment interferon-alpha, IL-2, G-CSF or GM-CSF); miscellaneous agents including platinum coordination complexes (in one embodiment oxaliplatin, cisplatin or carboplatin), anthracenediones (in one embodiment mitoxantrone), substituted urea (in one embodiment hydroxyurea), methylhydrazine derivatives including N-methylhydrazine (MIH) and procarbazine, adrenocortical suppressants (in one embodiment mitotane (o, p-DDD) or aminoglutethimide); hormones and antagonists including adrenocorticosteroid antagonists (in one embodiment prednisone and equivalents thereof), dexamethasone and aminoglutethimide; Gemzar™ (gemcitabine), progestin (in one embodiment hydroxyprogesterone caproate, medroxyprogesterone acetate or megestrol acetate); estrogen (in one embodiment diethylstilbestrol or ethinyl estradiol equivalents); antiestrogen (in one embodiment tamoxifen); androgens including testosterone propionate and fluoxymesterone/equivalents; antiandrogens (in one embodiment flutamide, gonadotropin-releasing hormone analogs and leuprolide); and non-steroidal antiandrogens (in one embodiment flutamide). Other chemotherapies applicable and preferred in certain embodiments of the combination therapy according to the invention are also outlined within the definition section of the present specification.

In one embodiment of a combination therapy according to the invention, the combination therapy of the antibody that binds to ANG-2 and the antibody that binds to PD-L1 is administered in a therapeutic treatment, wherein no additional chemotherapeutic agent is administered.

In one embodiment of a combination therapy according to the invention, the combination therapy of the antibody that binds to ANG-2, the antibody that binds to VEGF and the antibody that binds to PD-L1 is administered in a therapeutic treatment, wherein no additional chemotherapeutic agent is administered.

In one embodiment of a combination therapy according to the invention, the combination therapy of the antibody that binds to ANG-2 and the antibody that binds to PD-L1 is co-administered with a radiation therapy.

### 3. Specific Embodiments of the Invention

In the following specific embodiments of the invention are listed.
1. An antibody that binds to angiopoietin 2 (ANG-2), wherein the antibody is administered in a combination therapy with an antibody that binds to PD-L1, for use in

a) treating cancer,

b) delaying progression of cancer,

c) prolonging the survival of a patient suffering from cancer, or

d) stimulating a cell mediated immune response.
2. An antibody that binds to ANG-2, wherein the antibody is administered in combination therapy with an antibody that binds to VEGF and with an antibody that binds to PD-L1, for use in

a) treating cancer,

b) delaying progression of cancer,

c) prolonging the survival of a patient suffering from cancer, or

d) stimulating a cell mediated immune response.
3. An antibody that binds to ANG-2 and VEGF, wherein the antibody is administered in a combination therapy with an antibody that binds to PD-L1, for use in

a) treating cancer,

b) delaying progression of cancer,

c) prolonging the survival of a patient suffering from cancer; or

d) stimulating a cell mediated immune response.
4. The antibody that binds to ANG-2 for use according to embodiment 3, wherein the antibody that binds to ANG-2 and VEGF is bispecific.
5. The antibody that binds to ANG-2 for use according to any one of the preceding embodiments, wherein the antibody that binds to ANG-2 is a antibody or a humanized antibody.
6. The antibody that binds to ANG-2 for use according to any one of the preceding embodiments, wherein the antibody that binds to ANG-2 is of IgG isotype.
7. The antibody that binds to ANG-2 for use according to any one of the preceding embodiments, wherein the antibody that binds to ANG-2 specifically binds to human ANG-2 with a K_{D} value of less than 1.0×10⁻⁸ mol/l, as determined by surface plasmon resonance.
8. The antibody that binds to ANG-2 for use according to any one of the preceding embodiments, wherein the antibody that binds to ANG-2 inhibits the interaction of human ANG-2 with TIE2 receptor with an IC50 of 15 nM or less.
9. An antibody that binds to PD-L1, wherein the antibody is administered in a combination therapy with an antibody that binds to ANG-2, for use in

a) treating cancer,

b) delaying progression of cancer,

c) prolonging the survival of a patient suffering from cancer, or

d) stimulating a cell mediated immune response.
10. An antibody that binds to PD-L1, wherein the antibody is administered in a combination therapy with an antibody that binds to ANG-2 and an with antibody that binds to VEGF, for use in

a) treating cancer,

b) delaying progression of cancer,

c) prolonging the survival of a patient suffering from cancer, or

d) stimulating a cell mediated immune response.
11. An antibody that binds to PD-L1, wherein the antibody is administered in a combination therapy with an antibody that binds to ANG-2 and VEGF, for use in

a) treating cancer,

b) delaying progression of cancer,

c) prolonging the survival of a patient suffering from cancer, or

d) stimulating a cell mediated immune response.
12. The antibody that binds to PD-L1 for use according to embodiment 11, wherein the antibody that binds to ANG-2 and VEGF is bispecific.
13. The antibody that binds to PD-L1 for use according to any one of embodiments 9 to 12, wherein the antibody that binds to PD-L1 is a human antibody or a humanized antibody.
14. The antibody that binds to PD-L1 for use according to any one of embodiments 9 to 13, wherein the antibody that binds to PD-L1 is of IgG isotype.
15. The antibody that binds to PD-L1 for use according to any one of embodiments 9 to 14, wherein the antibody that binds to PD-L1 specifically binds to human PD-L1 with a K_{D} value of less than 1.0×10⁻⁸ mol/l, as determined by surface plasmon resonance.
16. The antibody that binds to PD-L1 for use according to any one of embodiments 9 to 15, wherein the antibody that binds to ANG-2 that is administered in the combination therapy inhibits the interaction of human ANG-2 with TIE2 receptor with an IC50 of 15 nM or less.
17. An antibody that binds to VEGF, wherein the antibody is administered in a combination therapy with an antibody that binds to ANG-2 and with an antibody that binds to PD-L1, for use in

a) treating cancer,

b) delaying progression of cancer,

c) prolonging the survival of a patient suffering from cancer, or

d) stimulating a cell mediated immune response.
18. The antibody that binds to VEGF for use according to embodiment 17, wherein the antibody that binds to VEGF is a human antibody or a humanized antibody.
19. The antibody that binds to VEGF for use according to any one of embodiments 17 or 18, wherein the antibody that binds to VEGF is of IgG isotype.

20. The antibody that binds to VEGF for use according to any one of embodiments 17 to 19, wherein the antibody that binds to VEGF specifically binds to human PD-L1 with a K_{D} value of less than 1.0×10⁻⁸ mol/l, as determined by surface plasmon resonance.

21. The antibody that binds to VEGF for use according to any one of embodiments 17 to 20, wherein the antibody that binds to ANG-2 that is administered in the combination therapy inhibits the interaction of human ANG-2 with TIE2 receptor with an IC50 of 15 nM or less.

22. The antibody that binds to ANG-2 for use according to any one of embodiments 1 to 8, or the antibody that binds to PD-L1 for use according to any one of embodiments 9 to 16, or the antibody that binds to VEGF for use according to any one of embodiments 17 to 21, wherein the cancer is selected from the group of lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, and lymphocytic leukemia.
23. The antibody that binds to ANG-2 for use according to any one of embodiments 1 to 8, or the antibody that binds to PD-L1 for use according to any one of embodiments 9 to 16, or the antibody that binds to VEGF for use according to any one of embodiments 17 to 21, wherein the antibody is for use in
a) treating a solid tumor,
b) delaying progression of a solid tumor, or
c) prolonging the survival of a patient suffering from a solid tumor.
24. The antibody that binds to ANG-2 for use according to any one of embodiments 1 to 8, and 22 to 23; or the antibody that binds to PD-L1 for use according to any one of embodiments 9 to 16 and 22 to 23; or the antibody that binds to VEGF for use according to any one of embodiments 17 to 21, and 22 to 23; wherein the antibody that binds to ANG-2 comprises the following variable domain amino acid sequences: variable heavy chain domain VH of SEQ ID NO:4, and variable light chain domain VL of SEQ ID NO: 5 (corresponding to the VH and VL domains of <ANG-2> LC06).
25. The antibody that binds to ANG-2 for use according to any one of embodiments 1 to 8, and 22 to 23; or the antibody that binds to PD-L1 for use according to any one of embodiments 9 to 16 and 22 to 23; or the antibody that binds to VEGF for use according to any one of embodiments 17 to 21, and 22 to 23; wherein the antibody that binds to ANG-2 comprises the following variable domain amino acid sequences: variable heavy chain domain VH of SEQ ID NO: 10, and variable light chain domain VL of SEQ ID NO: 5 (corresponding to the VH and VL domains of <ANG-2> E6Q).
26. The antibody that binds to ANG-2 for use according to any one of embodiments 1 to 8, and 22 to 25; or the antibody that binds to PD-L1 for use according to any one of embodiments 9 to 16 and 22 to 25; or the antibody that binds to VEGF for use according to any one of embodiments 17 to 21, and 22 to 25; wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
27. The antibody that binds to ANG-2 for use according to any one of embodiments 1 to 8, and 22 to 26; or the antibody that binds to PD-L1 for use according to any one of embodiments 9 to 16 and 22 to 26; or the antibody that binds to VEGF for use according to any one of embodiments 17 to 21, and 22 to 26; wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
28. The antibody that binds to ANG-2 for use according to any one of embodiments 2 to 8, and 22 to 27; or the antibody that binds to PD-L1 for use according to any one of embodiments 10 to 16 and 22 to 27; or the antibody that binds to VEGF for use according to any one of embodiments 17 to 21, and 22 to 27; wherein the antibody that binds to VEGF comprises the following variable domain amino acid sequences: variable heavy chain domain VH of SEQ ID NO: 8, and variable light chain domain VL of SEQ ID NO: 9 (corresponding to the VH and VL domains of <VEGF> bevacizumab).
29. The antibody that binds to ANG-2 for use according to any one of embodiments 3 to 8, and 22 to 28; or the antibody that binds to PD-L1 for use according to any one of embodiments 11 to 16 and 22 to 28; or the antibody that binds to VEGF for use according to any one of embodiments 17 to 21, and 22 to 28; wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 8, and a variable light chain domain VL of SEQ ID NO: 9 (corresponding to the VH and VL domains of <VEGF> bevacizumab).
30. The antibody that binds to ANG-2 for use according to any one of embodiments 3 to 8, and 22 to 29; or the antibody that binds to PD-L1 for use according to any one of embodiments 11 to 16 and 22 to 29; or the antibody that binds to VEGF for use according to any one of embodiments 17 to 21, and 22 to 29; wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
   wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
31. The antibody that binds to ANG-2 for use according to any one of embodiments 3 to 8, and 22 to 30; or the antibody that binds to PD-L1 for use according to any one of embodiments 11 to 16 and 22 to 30; or the antibody that binds to VEGF for use according to any one of embodiments 17 to 21, and 22 to 30; wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
   wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
32. The antibody that binds to ANG-2 for use according to any one of embodiments 2 to 8, and 22 to 27; or the antibody that binds to PD-L1 for use according to any one of embodiments 10 to 16 and 22 to 27; or the antibody that binds to VEGF for use according to any one of embodiments 17 to 21, and 22 to 27; wherein the antibody that binds to VEGF comprises the following variable domain amino acid sequences: variable heavy chain domain VH of SEQ ID NO: 6, and variable light chain domain VL of SEQ ID NO: 7 (corresponding to the VH and VL domains of <VEGF> B20.4.1).
33. The antibody that binds to ANG-2 for use according to any one of embodiments 3 to 8, and 22 to 27, and 32; or the antibody that binds to PD-L1 for use according to any one of embodiments 11 to 16 and 22 to 27, and 32; or the antibody that binds to VEGF for use according to any one of embodiments 17 to 21, and 22 to 27, and 32; wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1).
34. The antibody that binds to ANG-2 for use according to any one of embodiments 3 to 8, and 22 to 27, and 32 to 33; or the antibody that binds to PD-L1 for use according to any one of embodiments 11 to 16 and 22 to 27, and 32 to 33; or the antibody that binds to VEGF for use according to any one of embodiments 17 to 21, and 22 to 27, and 32 to 33; wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
   wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
35. The antibody that binds to ANG-2 for use according to any one of embodiments 3 to 8, and 22 to 27, and 32 to 34; or the antibody that binds to PD-L1 for use according to any one of embodiments 11 to 16 and 22 to 27, and 32 to 34; or the antibody that binds to VEGF for use according to any one of embodiments 17 to 21, and 22 to 27, and 32 to 34; wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
   wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
36. A method for
a) treating cancer,
b) delaying progression of cancer,
c) prolonging the survival of a patient suffering from cancer, or
d) stimulating a cell mediated immune response,
   wherein the method comprises the step of administering an effective amount of an antibody that binds to ANG-2 and an effective amount of an antibody that binds to PD-L1 to a patient in need thereof.
37. A method for
a) treating cancer,
b) delaying progression of cancer,
c) prolonging the survival of a patient suffering from cancer, or
d) stimulating a cell mediated immune response,
   wherein the method comprises the step of administering an effective amount of an antibody that binds to ANG-2, an effective amount of an antibody that binds to VEGF; and an effective amount of an antibody that binds to PD-L1 to a patient in need thereof.
38. A method for
a) treating cancer,
b) delaying progression of cancer,
c) prolonging the survival of a patient suffering from cancer, or
d) stimulating a cell mediated immune response,
   wherein the method comprises the step of administering an effective amount of an antibody that binds to ANG-2 and to VEGF; and an effective amount of an antibody that binds to PD-L1 to a patient in need thereof.
39. The method of embodiment 38, wherein the antibody that binds to ANG-2 and VEGF is bispecific.
40. The method according to any one of embodiments 36 to 39, wherein the cancer is selected from the group of lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, and lymphocytic leukemia.
41. The method according to any one of embodiments 36 to 39, wherein the method is for
a) treating a solid tumor,
b) delaying progression of a solid tumor, or
c) prolonging the survival of a patient suffering from a solid tumor.
42. The method according to any one of embodiments 36 to 41, wherein the antibody that binds to ANG-2 comprises the following variable domain amino acid sequences: variable heavy chain domain VH of SEQ ID NO:4, and variable light chain domain VL of SEQ ID NO: 5 (corresponding to the VH and VL domains of <ANG-2> LC06).
43. The method according to any one of embodiments 36 to 41, wherein the antibody that binds to ANG-2 comprises the following variable domain amino acid sequences: variable heavy chain domain VH of SEQ ID NO: 10, and variable light chain domain VL of SEQ ID NO: 5 (corresponding to the VH and VL domains of <ANG-2> E6Q).
44. The method according to any one of embodiments 36 to 43, wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
45. The method according to any one of embodiments 36 to 44, wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
46. The method according to any one of embodiments 36 to 45, wherein the antibody that binds to VEGF comprises the following variable domain amino acid sequences: variable heavy chain domain VH of SEQ ID NO: 8, and variable light chain domain VL of SEQ ID NO: 9 (corresponding to the VH and VL domains of <VEGF> bevacizumab).
47. The method according to any one of embodiments 36 to 46, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 8, and a variable light chain domain VL of SEQ ID NO: 9 (corresponding to the VH and VL domains of <VEGF> bevacizumab).
48. The method according to any one of embodiments 36 to 47, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
   wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
49. The method according to any one of embodiments 36 to 48, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
   wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
50. The method according to any one of embodiments 36 to 45, wherein the antibody that binds to VEGF comprises the following variable domain amino acid sequences: variable heavy chain domain VH of SEQ ID NO: 6, and variable light chain domain VL of SEQ ID NO: 7 (corresponding to the VH and VL domains of <VEGF> B20.4.1).
51. The method according to any one of embodiments 36 to 45 and 50, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1).
52. The method according to any one of embodiments 36 to 45 and 50 to 51, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
   wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
53. The method according to any one of embodiments 36 to 45 and 50 to 52, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
   wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
54. Use of an antibody that binds to ANG-2 for the manufacture of a medicament for
a) treating cancer,
b) delaying progression of cancer,
c) prolonging the survival of a patient suffering from cancer, or
d) stimulating a cell mediated immune response,
   wherein the antibody is for administration in a combination therapy with an antibody that binds to PD-L1.
55. Use of an antibody that binds to ANG-2 for the manufacture of a medicament for
a) treating cancer,
b) delaying progression of cancer,
c) prolonging the survival of a patient suffering from cancer, or
d) stimulating a cell mediated immune response,
   wherein the antibody is for administration in a combination therapy with an antibody that binds to VEGF and an antibody that binds to PD-L1.
56. Use of an antibody that binds to ANG-2 and VEGF for the manufacture of a medicament for
a) treating cancer,
b) delaying progression of cancer,
c) prolonging the survival of a patient suffering from cancer, or
d) stimulating a cell mediated immune response,
   wherein the antibody is for administration in a combination therapy with an antibody that binds to VEGF and an antibody that binds to PD-L1.
57. Use according to embodiment 56, wherein the antibody that binds to ANG-2 and VEGF is bispecific.
58. Use of an antibody that binds to PD-L1 for the manufacture of a medicament for
a) treating cancer,
b) delaying progression of cancer,
c) prolonging the survival of a patient suffering from cancer, or
d) stimulating a cell mediated immune response,
   wherein the antibody is for administration in a combination therapy with an antibody that binds to ANG-2.
59. Use of an antibody that binds to PD-L1 for the manufacture of a medicament for
a) treating cancer,
b) delaying progression of cancer,
c) prolonging the survival of a patient suffering from cancer, or
d) stimulating a cell mediated immune response,
   wherein the antibody is for administration in a combination therapy with an antibody that binds to ANG-2 and an antibody that binds to VEGF.
60. Use of an antibody that binds to PD-L1 for the manufacture of a medicament for
a) treating cancer,
b) delaying progression of cancer,
c) prolonging the survival of a patient suffering from cancer, or
d) stimulating a cell mediated immune response,
   wherein the antibody is for administration in a combination therapy with an antibody that binds to ANG-2 and VEGF.
61. Use according to embodiment 60, wherein the antibody that binds to ANG-2 and VEGF is bispecific.
62. Use of an antibody that binds to VEGF for the manufacture of a medicament for
a) treating cancer,
b) delaying progression of cancer,
c) prolonging the survival of a patient suffering from cancer, or
d) stimulating a cell mediated immune response,
   wherein the antibody is for administration in a combination therapy with an antibody that binds to ANG-2 and an antibody that binds to PD-L1.
63. The use according to any one of embodiments 54 to 62, wherein the cancer is selected from the group of lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, and lymphocytic leukemia.
64. The use according to any one of embodiments 54 to 62, wherein the method is for
a) treating a solid tumor,
b) delaying progression of a solid tumor, or
c) prolonging the survival of a patient suffering from a solid tumor.
65. The use according to any one of embodiments 54 to 64, wherein the antibody that binds to ANG-2 comprises the following variable domain amino acid sequences: variable heavy chain domain VH of SEQ ID NO:4, and variable light chain domain VL of SEQ ID NO: 5 (corresponding to the VH and VL domains of <ANG-2> LC06).
66. The use according to any one of embodiments 54 to 64, wherein the antibody that binds to ANG-2 comprises the following variable domain amino acid sequences: variable heavy chain domain VH of SEQ ID NO: 10, and variable light chain domain VL of SEQ ID NO: 5 (corresponding to the VH and VL domains of <ANG-2> E6Q).
67. The use according to any one of embodiments 64 to 66, wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
68. The use according to any one of embodiments 54 to 67, wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
69. The use according to any one of embodiments 54 to 68, wherein the antibody that binds to VEGF comprises the following variable domain amino acid sequences: variable heavy chain domain VH of SEQ ID NO: 8, and variable light chain domain VL of SEQ ID NO: 9 (corresponding to the VH and VL domains of <VEGF> bevacizumab).
70. The use according to any one of embodiments 54 to 69, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 8, and a variable light chain domain VL of SEQ ID NO: 9 (corresponding to the VH and VL domains of <VEGF> bevacizumab).
71. The use according to any one of embodiments 54 to 70, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
   wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
72. The use according to any one of embodiments 54 to 71, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
   wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
73. The use according to any one of embodiments 54 to 68, wherein the antibody that binds to VEGF comprises the following variable domain amino acid sequences: variable heavy chain domain VH of SEQ ID NO: 6, and variable light chain domain VL of SEQ ID NO: 7 (corresponding to the VH and VL domains of <VEGF> B20.4.1).
74. The use according to any one of embodiments 54 to 68 and 73, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1).
75. The use according to any one of embodiments 54 to 68 and 74 to 75, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
   wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
76. The use according to any one of embodiments 54 to 68 and 74 to 776, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
   wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.570”).
77. Use of an antibody that binds to ANG-2 and an antibody that binds to PD-L1 for the manufacture of a medicament for
a) treating cancer,
b) delaying progression of cancer,
c) prolonging the survival of a patient suffering from cancer, or
d) stimulating a cell mediated immune response.
78. Use of an antibody that binds to ANG-2 and an antibody that binds to VEGF for the manufacture of a medicament for
a) treating cancer,
b) delaying progression of cancer,
c) prolonging the survival of a patient suffering from cancer, or
d) stimulating a cell mediated immune response,
wherein the antibodies are for administration in a combination therapy with an antibody that binds to PD-L1.
79. Use of an antibody that binds to ANG-2, an antibody that binds to VEGF, and an antibody that binds to PD-L1 for the manufacture of a medicament for
a) treating cancer,
b) delaying progression of cancer,
c) prolonging the survival of a patient suffering from cancer, or
d) stimulating a cell mediated immune response.
80. Use of an antibody that binds to ANG-2 and VEGF, and an antibody that binds to PD-L1 for the manufacture of a medicament for
a) treating cancer,
b) delaying progression of cancer,
c) prolonging the survival of a patient suffering from cancer, or
d) stimulating a cell mediated immune response.
81. The use according to any one of embodiments 77 to 80, wherein the cancer is selected from the group of lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, and lymphocytic leukemia.
82. The use according to any one of embodiments 77 to 80, wherein the method is for
a) treating a solid tumor,
b) delaying progression of a solid tumor, or
c) prolonging the survival of a patient suffering from a solid tumor.
83. The use according to any one of embodiments 77 to 82, wherein the antibody that binds to ANG-2 comprises the following variable domain amino acid sequences: variable heavy chain domain VH of SEQ ID NO:4, and variable light chain domain VL of SEQ ID NO: 5 (corresponding to the VH and VL domains of <ANG-2> LC06).
84. The use according to any one of embodiments 77 to 82, wherein the antibody that binds to ANG-2 comprises the following variable domain amino acid sequences: variable heavy chain domain VH of SEQ ID NO: 10, and variable light chain domain VL of SEQ ID NO: 5 (corresponding to the VH and VL domains of <ANG-2> E6Q).
85. The use according to any one of embodiments 77 to 84, wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
86. The use according to any one of embodiments 77 to 85, wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
87. The use according to any one of embodiments 77 to 86, wherein the antibody that binds to VEGF comprises the following variable domain amino acid sequences: variable heavy chain domain VH of SEQ ID NO: 8, and variable light chain domain VL of SEQ ID NO: 9 (corresponding to the VH and VL domains of <VEGF> bevacizumab).
88. The use according to any one of embodiments 77 to 87, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 8, and a variable light chain domain VL of SEQ ID NO: 9 (corresponding to the VH and VL domains of <VEGF> bevacizumab).
89. The use according to any one of embodiments 77 to 88, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
   wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
90. The use according to any one of embodiments 77 to 89, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
   wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
91. The use according to any one of embodiments 77 to 86, wherein the antibody that binds to VEGF comprises the following variable domain amino acid sequences: variable heavy chain domain VH of SEQ ID NO: 6, and variable light chain domain VL of SEQ ID NO: 7 (corresponding to the VH and VL domains of <VEGF> B20.4.1).
92. The use according to any one of embodiments 77 to 86 and 91, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1).
93. The use according to any one of embodiments 77 to 86 and 91 to 92, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and a variable light chain domain VL of SEQ ID NO: 5; and
b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
   wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
94. The use according to any one of embodiments 77 to 86 and 91 to 93, wherein the antibody that binds to ANG-2 and VEGF comprises the following variable domain amino acid sequences:
c) the binding site or binding sites binding to ANG-2 comprise a variable heavy chain domain VH of SEQ ID NO: 10, and a variable light chain domain VL of SEQ ID NO: 5; and
d) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
   wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
95. The combination, antibody, method or use according to any one of the preceding embodiments, wherein the antibody that binds to ANG-2 (and optionally to VEGF) and the antibody that binds to PD-L1 are co-administered simultaneously.
96. The combination, antibody, method or use according to any one of the preceding embodiments, wherein the antibody that binds to ANG-2 (and optionally to VEGF) and the antibody that binds to PD-L1 are co-administered sequentially.
97. A pharmaceutical composition, comprising an antibody that binds to ANG-2, and an antibody that binds to PD-L1, wherein each antibody is formulated together with a pharmaceutically acceptable carrier.
98. The pharmaceutical composition according to embodiment 97, wherein the antibody that binds to ANG-2 and the antibody that binds to PD-L1 are formulated separately.
99. The pharmaceutical composition according to embodiment 97, wherein the antibody that binds to ANG-2 and the antibody that binds to PD-L1 are formulated together.
100. A pharmaceutical composition, comprising an antibody that binds to ANG-2, an antibody that binds to VEGF, and an antibody that binds to PD-L1, wherein each antibody is formulated together with a pharmaceutically acceptable carrier.
101. The pharmaceutical composition according to embodiment 100, wherein the antibody that binds to ANG-2 and the antibody that binds to VEGF are comprised in the form of one antibody that binds to ANG-2 and to VEGF.
102. The pharmaceutical composition according to embodiment 101, wherein the antibody that binds to ANG-2 and to VEGF is bispecific.
103. The pharmaceutical composition according to embodiment 100, wherein the antibody that binds to ANG-2, the antibody that binds to VEGF, and the antibody that binds to PD-L1 are formulated separately.
104. The pharmaceutical composition according to embodiment 101, wherein the antibody that binds to ANG-2, the antibody that binds to VEGF, and the antibody that binds to PD-L1 are formulated together.
105. The pharmaceutical composition according to embodiment 101 or 102, wherein the antibody that binds to ANG-2 and VEGF, and the antibody that binds to PD-L1 are formulated separately.
106. The pharmaceutical composition according to embodiment 97, wherein the antibody that binds to ANG-2 and VEGF, and the antibody that binds to PD-L1 are formulated together.
107. The pharmaceutical composition according to any one of embodiments 97 to 106, wherein the antibody that binds to ANG-2 comprises the following variable domain amino acid sequences: variable heavy chain domain VH of SEQ ID NO:4, and variable light chain domain VL of SEQ ID NO: 5 (corresponding to the VH and VL domains of <ANG-2> LC06).
108. The pharmaceutical composition according to any one of embodiments 97 to 106, wherein the antibody that binds to ANG-2 comprises the following variable domain amino acid sequences: variable heavy chain domain VH of SEQ ID NO: 10, and variable light chain domain VL of SEQ ID NO: 5 (corresponding to the VH and VL domains of <ANG-2> E6Q).
109. The pharmaceutical composition according to any one of embodiments 97 to 108, wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
110. The pharmaceutical composition according to any one of embodiments 97 to 109, wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
111. The pharmaceutical composition according to any one of embodiments 97 to 110, wherein the antibody that binds to VEGF comprises the following variable domain amino acid sequences: variable heavy chain domain VH of SEQ ID NO: 8, and variable light chain domain VL of SEQ ID NO: 9 (corresponding to the VH and VL domains of <VEGF> bevacizumab).
112. The pharmaceutical composition according to any one of embodiments 95 to 111, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 8, and a variable light chain domain VL of SEQ ID NO: 9 (corresponding to the VH and VL domains of <VEGF> bevacizumab).
113. The pharmaceutical composition according to any one of embodiments 97 to 112, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
      wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
114. The pharmaceutical composition according to any one of embodiments 97 to 113, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
      wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
115. The pharmaceutical composition according to any one of embodiments 97 to 110, wherein the antibody that binds to VEGF comprises the following variable domain amino acid sequences: variable heavy chain domain VH of SEQ ID NO: 6, and variable light chain domain VL of SEQ ID NO: 7 (corresponding to the VH and VL domains of <VEGF> B20.4.1).
116. The pharmaceutical composition according to any one of embodiments 97 to 110 and 115, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1).
117. The pharmaceutical composition according to any one of embodiments 97 to 110 and 115 to 116, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
      wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
118. The pharmaceutical composition according to any one of embodiments 97 to 110 and 115 to 116, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
      wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
119. A method for the manufacture of a pharmaceutical composition, comprising (a) formulating an antibody that binds to ANG-2 with a pharmaceutically acceptable carrier, and (b) formulating separately an antibody that binds to PD-L1 with a pharmaceutically acceptable carrier.
120. A method for the manufacture of a pharmaceutical composition, comprising formulating an antibody that binds to ANG-2 together with an antibody that binds to PD-L1 in combination with a pharmaceutically acceptable carrier.
121. A method for the manufacture of a pharmaceutical composition, comprising (a) formulating an antibody that binds to ANG-2 with a pharmaceutically acceptable carrier, (b) formulating separately an antibody that binds to VEGF with a pharmaceutically acceptable carrier and (c) formulating separately an antibody that binds to PD-L1 with a pharmaceutically acceptable carrier.
122. A method for the manufacture of a pharmaceutical composition, comprising (a) formulating an antibody that binds to ANG-2 together with an antibody that binds to VEGF in combination with a pharmaceutically acceptable carrier, and (b) formulating separately an antibody that binds to PD-L1 with a pharmaceutically acceptable carrier.
123. A method for the manufacture of a pharmaceutical composition, comprising formulating an antibody that binds to ANG-2 together with an antibody that binds to VEGF and together with an antibody that binds to PD-L1 in combination with a pharmaceutically acceptable carrier.
124. A method for the manufacture of a pharmaceutical composition, comprising (a) formulating an antibody that binds to ANG-2 and VEGF with a pharmaceutically acceptable carrier, and (b) formulating separately an antibody that binds to PD-L1 with a pharmaceutically acceptable carrier.
125. A method for the manufacture of a pharmaceutical composition, comprising formulating an antibody that binds to ANG-2 and VEGF together with an antibody that binds to PD-L1 in combination with a pharmaceutically acceptable carrier.
126. The method according to any one of embodiments 119 to 125, wherein the antibody that binds to ANG-2 comprises the following variable domain amino acid sequences: variable heavy chain domain VH of SEQ ID NO:4, and variable light chain domain VL of SEQ ID NO: 5 (corresponding to the VH and VL domains of <ANG-2> LC06).
127. The method according to any one of embodiments 119 to 125, wherein the antibody that binds to ANG-2 comprises the following variable domain amino acid sequences: variable heavy chain domain VH of SEQ ID NO: 10, and variable light chain domain VL of SEQ ID NO: 5 (corresponding to the VH and VL domains of <ANG-2> E6Q).
128. The method according to any one of embodiments 119 to 125, wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
129. The method according to any one of embodiments 119 to 128, wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
130. The method according to any one of embodiments 119 to 129, wherein the antibody that binds to VEGF comprises the following variable domain amino acid sequences: variable heavy chain domain VH of SEQ ID NO: 8, and variable light chain domain VL of SEQ ID NO: 9 (corresponding to the VH and VL domains of <VEGF> bevacizumab).
131. The method according to any one of embodiments 119 to 130, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 8, and a variable light chain domain VL of SEQ ID NO: 9 (corresponding to the VH and VL domains of <VEGF> bevacizumab).
132. The method according to any one of embodiments 119 to 131, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
      wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
133. The method according to any one of embodiments 119 to 132, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
      wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
134. The method according to any one of embodiments 119 to 129, wherein the antibody that binds to VEGF comprises the following variable domain amino acid sequences: variable heavy chain domain VH of SEQ ID NO: 6, and variable light chain domain VL of SEQ ID NO: 7 (corresponding to the VH and VL domains of <VEGF> B20.4.1).
135. The method according to any one of embodiments 119 to 129 and 134, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1).
136. The method according to any one of embodiments 119 to 129 and 134 to 135, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
      wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
137. The method according to any one of embodiments 119 to 129 and 134 to 136, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
      wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
138. An article of manufacture comprising (a) a container, a composition within the container comprising an antibody that binds to ANG-2, and (b) a package insert instructing the user of the composition to administer the antibody that binds to ANG-2 to a patient
   a. in the treatment of cancer,
   b. to delay progression of cancer,
   c. to prolong the survival of a patient suffering from cancer, or
   d. to stimulate a cell mediated immune response,
      wherein the administration of the antibody that binds to ANG-2 is in a combination therapy with an antibody that binds to PD-L1.
139. An article of manufacture comprising (a) a container, a composition within the container comprising an antibody that binds to ANG-2, and (b) a package insert instructing the user of the composition to administer the antibody that binds to ANG-2 to a patient
   a) in the treatment of cancer,
   b) to delay progression of cancer,
   c) to prolong the survival of a patient suffering from cancer, or
   d) to stimulate a cell mediated immune response,
      wherein the administration of the antibody that binds to ANG-2 is in a combination therapy with an antibody that binds to VEGF and an antibody that binds to PD-L1.
140. An article of manufacture comprising (a) a container, a composition within the container comprising an antibody that binds to PD-L1, and (b) a package insert instructing the user of the composition to administer the antibody that binds to PD-L1 to a patient
   a) in the treatment of cancer,
   b) to delay progression of cancer,
   c) to prolong the survival of a patient suffering from cancer, or
   d) to stimulate a cell mediated immune response,
      wherein the administration of the antibody that binds to PD-L1 is in a combination therapy with an antibody that binds to ANG-2.
141. An article of manufacture comprising (a) a container, a composition within the container comprising an antibody that binds to PD-L1, and (b) a package insert instructing the user of the composition to administer the antibody that binds to PD-L1 to a patient
   a) in the treatment of cancer,
   b) to delay progression of cancer,
   c) to prolong the survival of a patient suffering from cancer, or
   d) to stimulate a cell mediated immune response,
      wherein the administration of the antibody that binds to PD-L1 is in a combination therapy with an antibody that binds to ANG-2 and an antibody that binds to VEGF.
142. An article of manufacture comprising (a) a container, a composition within the container comprising an antibody that binds to VEGF, and (b) a package insert instructing the user of the composition to administer the antibody that binds to VEGF to a patient
   a) in the treatment of cancer,
   b) to delay progression of cancer,
   c) to prolong the survival of a patient suffering from cancer, or
   d) to stimulate a cell mediated immune response,
      wherein the administration of the antibody that binds to VEGF is in a combination therapy with an antibody that binds to ANG-2 and an antibody that binds to PD-L1.
143. An article of manufacture comprising (a) a container, a composition within the container comprising an antibody that binds to ANG-2, and (b) a container, a composition within the container comprising an antibody that binds to PD-L1, and (c) a package insert instructing the user of the composition to administer the antibody that binds to ANG-2 and the antibody that binds to PD-L1 to a patient
   a) in the treatment of cancer,
   b) to delay progression of cancer,
   c) to prolong the survival of a patient suffering from cancer, or
   d) to stimulate a cell mediated immune response.
144. An article of manufacture comprising (a) a container, a composition within the container comprising an antibody that binds to ANG-2, and (b) a container, a composition within the container comprising an antibody that binds to PD-L1, and (c) a package insert instructing the user of the composition to administer the antibody that binds to ANG-2 and the antibody that binds to PD-L1 to a patient
   a) in the treatment of cancer,
   b) to delay progression of cancer,
   c) to prolong the survival of a patient suffering from cancer, or
   d) to stimulate a cell mediated immune response,
      wherein the administration of the antibody that binds to ANG-2 and the antibody that binds to PD-L1 is in a combination therapy with an antibody that binds to VEGF.
145. An article of manufacture comprising (a) a container, a composition within the container comprising an antibody that binds to ANG-2, and (b) a container, a composition within the container comprising an antibody that binds to VEGF, and (c) a package insert instructing the user of the composition to administer the antibody that binds to ANG-2 and the antibody that binds to VEGF to a patient
   a. in the treatment of cancer,
   b. to delay progression of cancer,
   c. to prolong the survival of a patient suffering from cancer, or
   d. to stimulate a cell mediated immune response,
      wherein the administration of the antibody that binds to ANG-2 and the antibody that binds to VEGF is in a combination therapy with an antibody that binds to PD-L1.
146. The article of manufacture according to embodiment 145, comprising a container, a composition within said container comprising the antibody that binds to ANG-2 and the antibody that binds to VEGF.
147. The article of manufacture according to embodiment 145 or 146, comprising a container, a composition within said container comprising the antibody that binds to ANG-2 and the antibody that binds to VEGF in the form of an antibody that binds to ANG-2 and to VEGF.
148. The article of manufacture according to embodiment 147, comprising a container, a composition within said container comprising the antibody that binds to ANG-2 and the antibody that binds to VEGF in the form of a bispecific antibody that binds to ANG-2 and to VEGF.
149. An article of manufacture comprising (a) a container, a composition within the container comprising an antibody that binds to ANG-2, and (b) container, a composition within the container comprising an antibody that binds to VEGF, and (c) a container, a composition within the container comprising an antibody that binds to PD-L1, and (d) a package insert instructing the user of the composition to administer the antibodies that bind to ANG-2, VEGF, and PD-L1, respectively, in a combination therapy to a patient
   a. in the treatment of cancer,
   b. to delay progression of cancer,
   c. to prolong the survival of a patient suffering from cancer, or
   d. to stimulate a cell mediated immune response.
150. The article of manufacture according to embodiment 149, comprising a container, a composition within said container comprising the antibody that binds to ANG-2 and the antibody that binds to VEGF.
151. The article of manufacture according to embodiment 149 or 150, comprising a container, a composition within said container comprising the antibody that binds to ANG-2 and the antibody that binds to VEGF in the form of an antibody that binds to ANG-2 and to VEGF.
152. The article of manufacture according to embodiment 151, comprising a container, a composition within said container comprising the antibody that binds to ANG-2 and the antibody that binds to VEGF in the form of a bispecific antibody that binds to ANG-2 and to VEGF.
153. The article of manufacture according to any one of embodiments 138 to 152, wherein the cancer is selected from the group of lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, and lymphocytic leukemia.
154. The article of manufacture according to any one of embodiments 138 to 152, wherein the method is for
   a) treating a solid tumor,
   b) delaying progression of a solid tumor, or
   c) prolonging the survival of a patient suffering from a solid tumor.
155. The article of manufacture according to any one of embodiments 138 to 154, wherein the antibody that binds to ANG-2 comprises the following variable domain amino acid sequences: variable heavy chain domain VH of SEQ ID NO:4, and variable light chain domain VL of SEQ ID NO: 5 (corresponding to the VH and VL domains of <ANG-2> LC06).
156. The article of manufacture according to any one of embodiments 138 to 154, wherein the antibody that binds to ANG-2 comprises the following variable domain amino acid sequences: variable heavy chain domain VH of SEQ ID NO: 10, and variable light chain domain VL of SEQ ID NO: 5 (corresponding to the VH and VL domains of <ANG-2> E6Q).
157. The article of manufacture according to any one of embodiments 138 to 156, wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
158. The article of manufacture according to any one of embodiments 138 to 157, wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
159. The article of manufacture according to any one of embodiments 138 to 158, wherein the antibody that binds to VEGF comprises the following variable domain amino acid sequences: variable heavy chain domain VH of SEQ ID NO: 8, and variable light chain domain VL of SEQ ID NO: 9 (corresponding to the VH and VL domains of <VEGF> bevacizumab).
160. The article of manufacture according to any one of embodiments 138 to 159, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 8, and a variable light chain domain VL of SEQ ID NO: 9 (corresponding to the VH and VL domains of <VEGF> bevacizumab).
161. The article of manufacture according to any one of embodiments 138 to 160, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
      wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
162. The article of manufacture according to any one of embodiments 138 to 161, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 8, and variable light chain domain VL of SEQ ID NO: 9 (corresponding to the VH and VL domains of <VEGF> bevacizumab); and
      wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).
163. The article of manufacture according to any one of embodiments 138 to 158, wherein the antibody that binds to VEGF comprises the following variable domain amino acid sequences: variable heavy chain domain VH of SEQ ID NO: 6, and variable light chain domain VL of SEQ ID NO: 7 (corresponding to the VH and VL domains of <VEGF> B20.4.1).
164. The article of manufacture according to any one of embodiments 138 to 158 and 163, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1).
165. The article of manufacture according to any one of embodiments 138 to 158 and 163 and 164, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
      wherein the antibody that binds to PD-L1 comprises variable domain amino acid sequences, selected from the group of:
   variable heavy chain domain VH of SEQ ID NO: 17, and variable light chain domain VL of SEQ ID NO: 20 (corresponding to the VH and VL domains of <PD-L1> “243.55.S70” as disclosed herein);
   variable heavy chain domain VH of SEQ ID NO: 18, and variable light chain domain VL of SEQ ID NO: 21 (corresponding to the VH and VL domains of <PD-L1> “243.55.H1” as disclosed herein);
   variable heavy chain domain VH of SEQ ID NO: 18, and variable light chain domain VL of SEQ ID NO: 22 (corresponding to the VH and VL domains of <PD-L1> “243.55.H12” as disclosed herein);
   variable heavy chain domain VH of SEQ ID NO: 18, and variable light chain domain VL of SEQ ID NO: 23 (corresponding to the VH and VL domains of <PD-L1> “243.55.H37” as disclosed herein);
   variable heavy chain domain VH of SEQ ID NO: 18, and variable light chain domain VL of SEQ ID NO: 24 (corresponding to the VH and VL domains of <PD-L1> “243.55.H70” as disclosed herein);
   variable heavy chain domain VH of SEQ ID NO: 18, and variable light chain domain VL of SEQ ID NO: 25 (corresponding to the VH and VL domains of <PD-L1> “243.55.H89” as disclosed herein);
   variable heavy chain domain VH of SEQ ID NO: 18, and variable light chain domain VL of SEQ ID NO: 26 (corresponding to the VH and VL domains of <PD-L1> “243.55.S1” as disclosed herein);
   variable heavy chain domain VH of SEQ ID NO: 18, and variable light chain domain VL of SEQ ID NO: 27 (corresponding to the VH and VL domains of <PD-L1> “243.55.5” as disclosed herein);
   variable heavy chain domain VH of SEQ ID NO: 18, and variable light chain domain VL of SEQ ID NO: 28 (corresponding to the VH and VL domains of <PD-L1> “243.55.8” as disclosed herein);
   variable heavy chain domain VH of SEQ ID NO: 18, and variable light chain domain VL of SEQ ID NO: 29 (corresponding to the VH and VL domains of <PD-L1> “243.55.30” as disclosed herein);
   variable heavy chain domain VH of SEQ ID NO: 18, and variable light chain domain VL of SEQ ID NO: 30 (corresponding to the VH and VL domains of <PD-L1> “243.55.34” as disclosed herein);
   variable heavy chain domain VH of SEQ ID NO: 18, and variable light chain domain VL of SEQ ID NO: 31 (corresponding to the VH and VL domains of <PD-L1> “243.55.S37” as disclosed herein);
   variable heavy chain domain VH of SEQ ID NO: 18, and variable light chain domain VL of SEQ ID NO: 32 (corresponding to the VH and VL domains of <PD-L1> “243.55.49” as disclosed herein);
   variable heavy chain domain VH of SEQ ID NO: 18, and variable light chain domain VL of SEQ ID NO: 33 (corresponding to the VH and VL domains of <PD-L1> “243.55.51” as disclosed herein);
   variable heavy chain domain VH of SEQ ID NO: 18, and variable light chain domain VL of SEQ ID NO: 34 (corresponding to the VH and VL domains of <PD-L1> “243.55.62” as disclosed herein); and
   variable heavy chain domain VH of SEQ ID NO: 19, and variable light chain domain VL of SEQ ID NO: 35 (corresponding to the VH and VL domains of <PD-L1> “243.55.84” as disclosed herein).
166. The article of manufacture according to any one of embodiments 138 to 158 and 163 and 165, wherein the antibody that binds to ANG-2 and VEGF comprises the following variable domain amino acid sequences:
   a) the binding site or binding sites binding to ANG-2 comprise a variable heavy chain domain VH of SEQ ID NO: 10, and a variable light chain domain VL of SEQ ID NO: 5; and
   b) the binding site or binding sites binding to VEGF comprise a variable heavy chain domain VH of SEQ ID NO: 6, and a variable light chain domain VL of SEQ ID NO: 7 (corresponding to the VH and VL domains of <VEGF> B20.4.1); and
      wherein the antibody that binds to PD-L1 comprises the following variable domain amino acid sequences: variable heavy chain domain VH of SEQ ID NO: 17, and variable light chain domain VL of SEQ ID NO: 20 (corresponding to the VH and VL domains of <PD-L1> “243.55.S70”).

### Description of the Amino Acid Sequences

| | |
|---|---|
| SEQ ID NO: 1 | human angiopoietin 2 |
| SEQ ID NO: 2 | human vascular endothelial growth factor |
| SEQ ID NO: 3 | human programmed death ligand 1 |
| SEQ ID NO: 4 | variable heavy chain domain VH of <ANG-2> LC06 |
| SEQ ID NO: 5 | variable light chain domain VL of <ANG-2> LC06 and E6Q |
| SEQ ID NO: 6 | variable heavy chain domain VH of <VEGF> B20.4.1 |
| SEQ ID NO: 7 | variable light chain domain VL of <VEGF> B20.4.1 |
| SEQ ID NO: 8 | variable heavy chain domain VH of <VEGF> bevacizumab |
| SEQ ID NO: 9 | variable light chain domain VL of <VEGF> bevacizumab |
| SEQ ID NO: 10 | variable heavy chain domain VH of <ANG-2> E6Q |
| SEQ ID NO: 11 | ANG-2 heavy chain of <ANG-2/VEGF> E6Q/B20.4.1 and |
| | <ANG-2/VEGF> E6Q/bevacizumab |
| SEQ ID NO: 12 | ANG-2 light chain of <ANG-2/VEGF> E6Q/B20.4.1 and |
| | <ANG-2/VEGF> E6Q/bevacizumab |
| SEQ ID NO: 13 | VEGF heavy chain of <ANG-2/VEGF> E6Q/B20.4.1 |
| SEQ ID NO: 14 | VEGF light chain of <ANG-2/VEGF> E6Q/B20.4.1 |
| SEQ ID NO: 15 | VEGF heavy chain of <ANG-2/VEGF> E6Q/bevacizumab |
| SEQ ID NO: 16 | VEGF light chain of <ANG-2/VEGF> E6Q/bevacizumab |
| SEQ ID NO: 17 | variable heavy chain domain VH of <PD-L1> 243.55 variant 1 |
| SEQ ID NO: 18 | variable heavy chain domain VH of <PD-L1> 243.55 variant 2 |
| SEQ ID NO: 19 | variable heavy chain domain VH of <PD-L1> 243.55 variant 3 |
| SEQ ID NO: 20 | variable light chain domain VL of <PD-L1> 243.55 variant 1 |
| SEQ ID NO: 21 | variable light chain domain VL of <PD-L1> 243.55 variant 2 |
| SEQ ID NO: 22 | variable light chain domain VL of <PD-L1> 243.55 variant 3 |
| SEQ ID NO: 23 | variable light chain domain VL of <PD-L1> 243.55 variant 4 |
| SEQ ID NO: 24 | variable light chain domain VL of <PD-L1> 243.55 variant 5 |
| SEQ ID NO: 25 | variable light chain domain VL of <PD-L1> 243.55 variant 6 |
| SEQ ID NO: 26 | variable light chain domain VL of <PD-L1> 243.55 variant 7 |
| SEQ ID NO: 27 | variable light chain domain VL of <PD-L1> 243.55 variant 8 |
| SEQ ID NO: 28 | variable light chain domain VL of <PD-L1> 243.55 variant 9 |
| SEQ ID NO: 29 | variable light chain domain VL of <PD-L1> 243.55 variant 10 |
| SEQ ID NO: 30 | variable light chain domain VL of <PD-L1> 243.55 variant 11 |
| SEQ ID NO: 31 | variable light chain domain VL of <PD-L1> 243.55 variant 12 |
| SEQ ID NO: 32 | variable light chain domain VL of <PD-L1> 243.55 variant 13 |
| SEQ ID NO: 33 | variable light chain domain VL of <PD-L1> 243.55 variant 14 |
| SEQ ID NO: 34 | variable light chain domain VL of <PD-L1> 243.55 variant 15 |
| SEQ ID NO: 35 | variable light chain domain VL of <PD-L1> 243.55 variant 16 |

### EXAMPLES

The following examples are provided to aid the understanding of the present invention, the true scope of which is set forth in the appended claims. It is understood that modifications can be made in the procedures set forth without departing from the spirit of the invention.

The following antibodies were used within the examples section or are disclosed and referred to herein:

TABLE 1a
Amino acid sequences in SEQ ID NO: of VH, VL of
indicated antibodies

| antibody | VH | VL |
|---|---|---|
| <ANG-2> LC06 | 4 | 5 |
| <ANG-2> E6Q | 10 | 5 |
| <VEGF> B20.4.1 | 6 | 7 |
| <VEGF> bevacizumab | 8 | 9 |
| <ANG-2/VEGF> E6Q/B20.4.1 | ANG-2: 10 | ANG-2: 5 |
| | VEGF: 6 | VEGF: 7 |
| <ANG-2/VEGF> E6Q/bevacizumab | ANG-2: 10 | ANG-2: 5 |
| (corresponding to “xMab1” of | VEGF: 8 | VEGF: 9 |
| WO 2011/117329) | | |
| <PD-L1> “243.55.S70” | 17 | 20 |
| <PD-L1> “243.55.H1” | 18 | 21 |
| <PD-L1> “243.55.H12” | 18 | 22 |
| <PD-L1> “243.55.H37” | 18 | 23 |
| <PD-L1> “243.55.H70” | 18 | 24 |
| <PD-L1> “243.55.H89” | 18 | 25 |
| <PD-L1> “243.55.S1” | 18 | 26 |
| <PD-L1> “243.55.5” | 18 | 27 |
| <PD-L1> “243.55.8” | 18 | 28 |
| <PD-L1> “243.55.30” | 18 | 29 |
| <PD-L1> “243.55.34” | 18 | 30 |
| <PD-L1> “243.55.S37” | 18 | 31 |
| <PD-L1> “243.55.49” | 18 | 32 |
| <PD-L1> “243.55.51” | 18 | 33 |
| <PD-L1> “243.55.62” | 18 | 34 |
| <PD-L1> “243.55.84” | 19 | 35 |

TABLE 1b
Amino acid sequences in SEQ ID NO: of heavy chain
and light chain of indicated antibodies

| | heavy chain | light chain | heavy chain | light chain |
|---|---|---|---|---|
| antibody | ANG-2 | ANG-2 | VEGF | VEGF |
| <ANG-2/VEGF> | 11 | 12 | 13 | 14 |
| E6Q/B20.4.1 | | | | |
| <ANG-2/VEGF> | 11 | 12 | 15 | 16 |
| E6Q/bevacizumab | | | | |
| (corresponding to | | | | |
| “xMab1” of | | | | |
| WO 2011/117329) | | | | |

Antibody “<ANG-2> E6Q” that Binds to ANG-2

Within the experiments, a bispecific antibody binding to ANG-2 and VEGF was used (<ANG-2/VEGF> E6Q/B20.4.1), which comprises an ANG-2 binding site with a VH of SEQ ID NO: 10 and a VL of SEQ ID NO: 5 (variable domains of <ANG-2> E6Q). This binding site is derived from antibody <ANG-2> LC06, which is disclosed in WO2010/069532 (and which is therein referred to as <ANG-2>Ang2i_LC06). The variable domains of <ANG-2> LC06 and <ANG-2> E6Q differ in only one amino acid mutation at position 6 (i.e. an E6Q substitution) in the VH domain.

### Use of Mouse-Crossreactive Surrogate Antibodies

It is to be noted that the VEGF binding site of the anti-human-VEGF antibody bevacizumab is not mouse-crossreactive. Hence, to carry out the experiment in mice, a mouse-crossreactive surrogate antibody (herein referred to as <VEGF> B20-4.1) was used for the in vivo experiments. To realize this, the surrogate antibody was generated by replacing only the VH and VL domains of bevacizumab by the mouse-crossreactive VEGF-binding VH and VL domains of <VEGF> B20-4.1. For this reason, the bispecific antibody that binds to ANG-2 and VEGF used in the experiment comprised the binding arm of <VEGF> B20-4.1. The corresponding bispecific antibody including the VH and VL domains of bevacizumab is the antibody referred to herein as <ANG-2/VEGF> E6Q/bevacizumab (which is, e.g., disclosed in WO 2011/117329 as antibody “xMab1”).

In addition, the antibody that bind to human PD-L1 “243.55.S70”, “243.55.H1”, “243.55.H12”, “243.55.H37”, “243.55.H70”, “243.55.H89”, “243.55.51”, “243.55.5”, “243.55.8”, “243.55.30”, “243.55.34”, “243.55.S37”, “243.55.49”, “243.55.51”, “243.55.62”, and “243.55.84” are not mouse-crossreactive. Hence, the VH and VL domains of said anti-human-PD-L1 antibodies were replaced by the VH and VL domains of a mouse-crossreactive anti-PD-L1 antibody that is referred to herein as <PD-L1> 6E11 (Genentech). Antibody <PD-L1> 6E11 (human IgG1) was derived from anti-PD-L1 antibody clone 25A1 (mIgG2a, D265A, and N297A; Genentech, disclosed in Junttila et al. Cancer Res. 2014 Oct. 1; 74(19):5561-71), which was obtained from immunization of Pdl1−/−mice with a PD-L1 -Fc fusion protein and which was further cloned onto a murine IgG2a isotype and modified with previously described mutations abolishing binding to Fcγ receptors (Shields et al., J. Biol. Chem. 276, 6591-6604).

The experiments as described herein use (non-humanized) Balb/c mice. In similar experiments using transgenic humanized mice, as well as in clinical trials, conducted using not the surrogate antibodies, but instead human-VEGF reactive (and not mouse-crossreactive) bevacizumab or the bispecific antibody that binds to ANG-2 and VEGF <ANG-2/VEGF> E6Q/bevacizumab (which is, e.g., disclosed in WO 2011/117329 as antibody “xMab1”), similar results are expected based on the same mechanism of action. The same applies for the use of human-PD-L1 reactive antibodies comprising the VH and VL domains of anti-PD-L1 antibodies “243.55.570”, “243.55.H1”, “243.55.H12”, “243.55.H37”, “243.55.H70”, “243.55.H89”, “243.55.S1”, “243.55.5”, “243.55.8”, “243.55.30”, “243.55.34”, “243.55.S37”, “243.55.49”, “243.55.51”, “243.55.62”, or “243.55.84”, instead of the use of the surrogate antibody <PD-L1> 6E11 in transgenic humanized mice or clinical trials.

### Example 1A

### Inhibition of the Interaction of Human ANG-2 with TIE2 Receptor of Antibody that Binds to ANG-2 “<ANG2> LC06”

Blocking of human ANG-2/human Tie2 interaction was shown by receptor interaction ELISA. 384-well Maxisorp plates (Nunc) were coated with 0.5 μg/ml human Tie2 (R&D Systems, UK, Cat. No.313-TI or in house produced material) for 2 h at room temperature and blocked with PBS supplemented with 0.2% Tween-20 and 2% BSA (Roche Diagnostics GmbH, DE) for 1 h at room temperature under shaking. In the meantime, dilutions of the purified antibody in PBS were incubated together with 0.2 μg/ml huAngiopoietin-1/2 (R&D Systems #923-AN/CF, R&D Systems, UK, Cat. No. 623-AN or in house produced material) for 1 hour at RT. After washing a mixture of 0.5 μg/ml biotinylated anti-Angiopoietin-1/2 clone (R&D Systems #BAF923, BAM0981 R&D Systems, UK) and 1:3000 diluted streptavidin HRP (Roche Diagnostics GmbH, DE, Cat. No. 11089153001) was added for 1 h. Thereafter the plates were washed 6 times with PBST. Plates were developed with freshly prepared ABTS reagent (Roche Diagnostics GmbH, DE, buffer #204 530 001, tablets #11 112 422 001) for 30 minutes at RT. Absorbance was measured at 405 nm.

The obtained inhibitory concentration is indicated in Table 2.

TABLE 2
IC50 of antibody <ANG-2> LC06 as determined
by ANG-2/Tie2 interaction ELISA

| Antibody | Inhibitory concentration |
|---|---|
| LC06 | 0.1 nM |

### Example 1B

### Inhibition of the Interaction of Human ANG-2 with TIE2 Receptor of Bispecific Antibody that Binds to ANG-2 and VEGF “<ANG-2/VEGF> E6Q/B20.4.1”

The interaction ELISA was performed on 384 well microtiter plates (MicroCoat, DE, Cat. No. 464718) at RT. After each incubation step plates were washed 3 times with PBST. ELISA plates were coated with 5 μg/ml Tie-2 protein for 1 hour (h). Thereafter the wells were blocked with PBS supplemented with 0.2% Tween-20 and 2% BSA (Roche Diagnostics GmbH, DE) for 1 h. Dilutions of purified bispecific Xmab antibodies in PBS were incubated together with 0.2 μg/ml huAngiopoietin-2 (R&D Systems, UK, Cat. No. 623-AN) for 1 h at RT. After washing a mixture of 0.5 μg/ml biotinylated anti-Angiopoietin-2 clone BAM0981 (R&D Systems, UK) and 1:3000 diluted streptavidin HRP (Roche Diagnostics GmbH, DE, Cat. No. 11089153001) was added for 1 h. Thereafter the plates were washed 3 times with PBST. Plates are developed with freshly prepared ABTS reagent (Roche Diagnostics GmbH, DE, buffer #204 530 001, tablets #11 112 422 001) for 30 minutes at RT. Absorbance was measured at 405 nm and the IC50 was determined.

The obtained IC50 is indicated in Table 3.

TABLE 3
IC50 of antibody <ANG-2/VEGF> E6Q/B20.4.1
as determined by ANG-2/Tie2 interaction ELISA

| Antibody | Inhibitory concentration |
|---|---|
| E6Q/B20.4.1 | 12 nM |

### Example 2

### In Vivo Anti-Tumor Efficacy of a Combination Therapy according to the Invention including Administration of an Antibody that Binds to ANG-2 and an Antibody that Binds to PD-L1

### Methods

Test agents: Indicated antibodies were generated at Roche Diagnostics GmbH, Penzberg, Germany, except for antibody <PD-L1> 6E11, which was obtained from Genentech, USA. Antibody buffer included 20 mM histidine and 140 mM sodium chloride (pH 6.0). Antibody solutions were diluted appropriately in the above mentioned buffer from stock prior to administrations.
Cell lines and culture conditions: The murine CT26WT cell line was routinely cultured in RPMI 1640 supplemented with 10% fetal bovine serum (PAA Laboratories, Austria) and 2 mM L-glutamine at 37° C. in a water-saturated atmosphere at 5% CO₂.
Animals: Female Balb/c mice aged 6-7 weeks at arrival (purchased from Charles River, Sulzfeld, Germany) were maintained under specific-pathogen-free condition with daily cycles of 12 h light/12 h darkness according to committed guidelines (GV-Solas; Felasa; TierschG). Experimental study protocol was reviewed and approved by local government (Regierung von Oberbayern; registration no. 55.2-1-54-2531.2-32-10). After arrival animals were maintained in the animal facility for one week to get accustomed to new environment and for observation. Continuous health monitoring was carried out on regular basis. Diet food (Altromin) and water (filtered) were provided ad libitum.
Monitoring: Animals were controlled daily for clinical symptoms and detection of adverse effects. For monitoring throughout the experiment body weight of animals was documented two times weekly and tumor volume was measured by caliper after randomization.
Tumor cell inoculation: The mice were s.c. injected with 1.000.000 syngeneic CT26WT tumor cells per mouse and treatment began on day 12 when tumors reached a size of about 120 mm³.
   Treatment of Animals with Combination Therapy:

As a negative control, Histidin buffer (20 mM Histidin, 140 mM NaCl. ph 6.0) was applied to the test animals. As comparative analyses monospecific antibody <PD-L1> 6E11, and bispecific antibody <ANG-2/VEGF> E6Q/B20.4.1 were run in parallel.

Mice were euthanized according to veterinary regulations when tumors reached a size of 1200 mm³ (endpoint). Following dosages and treatment schedules have been applied (all antibodies were applied intraperitoneally):

TABLE 4
Treatment schedule

| | Dose | |
|---|---|---|
| Therapy | [mg/kg] | Treatment schedule |
| Histidin buffer | — | once weekly |
| | | (2x: days 12/19) |
| <ANG-2/VEGF> | 10 | once weekly |
| E6Q/B20.4.1 | | (2x: days 12/19) |
| <PD-L1> 6E11 | initially 10, | every 3 days |
| | followed by 5 | (6x: days 12/15/18/21/24/27) |
| <PD-L1> 6E11 | initially 10, | every 3 days |
| with | followed by 5 + | (6x: days 12/15/18/21/24/27) + |
| <ANG-2/VEGF> | 10 | once weekly |
| E6Q/B20.4.1 | | (8x: days 12/19/26/33/40/47/54/ |
| | | 61) |

### Results

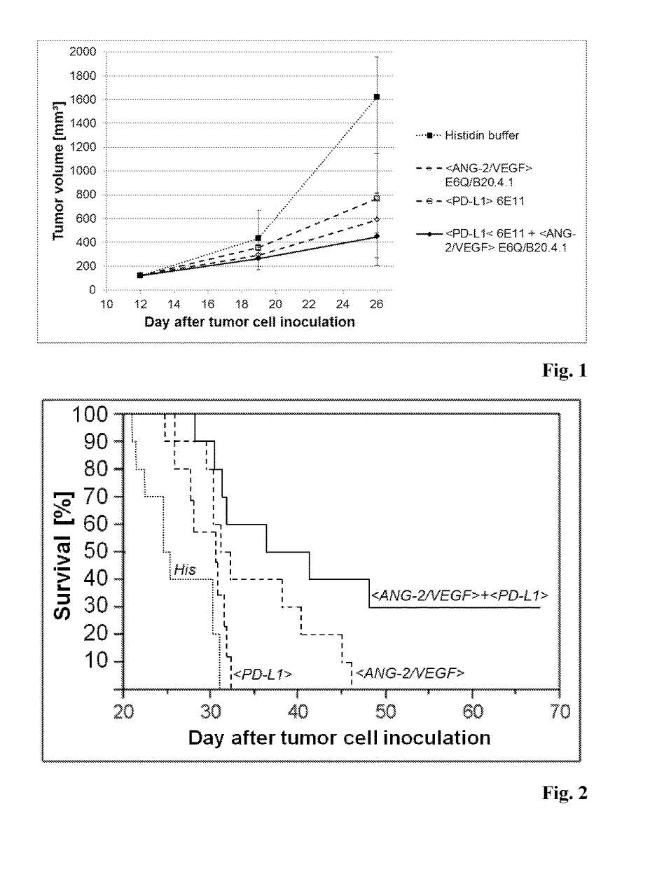

Results of tumor volume/tumor growth inhibition are shown in FIG. 1.

Results of overall survival of the treated individuals are shown in FIG. 2.

The data indicate that treatment with a combination therapy of antibodies that bind to PD-L1 and ANG-2/VEGF prolonged the overall survival prominently when compared to the control group, as well as the groups treated with a monotherapy of anti-PD-L1. The median time-to-event for the combination therapy of anti-ANG-2/VEGF and anti-PD-L1 was 39 days compared to 25 days within the control group, and 30 days within the group treated with a anti-PD-L1 monotherapy. From the animals that survived under the combination therapy of anti-ANG-2/VEGF with anti-PD-L1, one individual was tumor free at day 89.

In addition, a synergistic effect of the PD-L1 and ANG-2/VEGF combination therapy on tumor growth inhibition could be observed. Note that tumor growth inhibition indicated in FIG. 1 was only observed until day 26, the day, when the first individual in the vehicle group had to be sacrificed. However, overall survival data and the analysis of the surviving test animals indicate tumor free individuals only in the group receiving the PD-L1 and ANG-2/VEGF combination therapy.

1. A method for treating a cancer patient, delaying progression of cancer in a patient, prolonging the survival of a patient suffering from cancer, or stimulating a cell-mediated immune response in a patient, the method comprising administering to the patient an antibody that binds to angiopoietin 2 (ANG-2), wherein the antibody is administered as part of a combination therapy with an antibody that binds to programmed death ligand (PD-L1).

2. The method according to claim 1, wherein the antibody binds to ANG-2 and to VEGF.

3. The method according to claim 2, wherein the antibody that binds to ANG-2 and VEGF is a bispecific antibody.

4. The method according to claim 1, wherein the antibody is administered in combination therapy with an antibody that binds to VEGF and with an antibody that binds to (PD-L1).

5. The method according to claim 1 wherein the antibody that binds to ANG-2 comprises the following variable domain amino acid sequences: (a) variable heavy chain domain VH of SEQ ID NO:4, and variable light chain domain VL of SEQ ID NO: 5, or (b) variable heavy chain domain VH of SEQ ID NO: 10, and variable light chain domain VL of SEQ ID NO: 5.

6. The method according to claim 2, wherein the antibody that binds to VEGF comprises the following variable domain amino acid sequences: variable heavy chain domain VH of SEQ ID NO: 8, and variable light chain domain VL of SEQ ID NO: 9.

7. The method according to claim 2, wherein the antibody that binds to ANG-2 and VEGF comprises the following variable domain amino acid sequences:
a) the binding site or binding sites binding to ANG-2 comprise a variable heavy chain domain VH of SEQ ID NO: 10, and variable light chain domain VL of SEQ ID NO: 5; and
b) the binding site or binding sites binding to VEGF comprise a variable heavy chain domain VH of SEQ ID NO: 8, and a variable light chain domain VL of SEQ ID NO: 9.

8. The method according to claim 1, wherein the antibody that binds to PD-L1 comprises the following variable domain amino acid sequences: variable heavy chain domain VH of SEQ ID NO: 17, and variable light chain domain VL of SEQ ID NO: 20.

9. The method according to claim 1, wherein the method is to
a) treat a solid tumor,
b) delay progression of a solid tumor, or
c) prolong the survival of a patient suffering from a solid tumor.

10.-13. (canceled)

14. The method according to claim 1, wherein the antibody that binds to ANG 2 and the antibody that binds to PD-L1 are co-administered simultaneously.

15. The method according to claim 1, wherein the antibody that binds to ANG 2 and the antibody that binds to PD-L1 are co-administered sequentially.

16. A pharmaceutical composition, comprising an antibody that binds to ANG-2, and an antibody that binds to PD-L1, wherein each antibody is formulated together with a pharmaceutically acceptable carrier.

17. The pharmaceutical composition according to claim 16, wherein the antibody that binds to ANG-2 binds to ANG-2 and binds to VEGF.

18. The pharmaceutical composition according to claim 17, wherein the antibody that binds to ANG-2 and to VEGF is bispecific.

19. A pharmaceutical composition comprising an antibody that binds to ANG-2, and an antibody that binds to PD-L1, wherein each antibody is is formulated separately.
